(19) United States
(12) Patent Application Publication
(10) Pub. No.: US 2026/0114487 A1
(43) Pub. Date: Apr. 30, 2026

(54) KIDNEY SPECIFIC FASTING-MIMICKING DIET INDUCES PODOCYTE REPROGRAMMING AND RESTORES RENAL FUNCTION IN GLOMERULOPATHY

(71) Applicants: University of Southern California, Los Angeles, CA (US); Children's Hospital Los Angeles, Los Angeles, CA (US); Sapienza University of Rome, Rome (IT)

(72) Inventors: Valter D. LONGO, Playa del Rey, CA (US); Alessandro LAVIANO, Rome (IT); Silvia LAI, Rome (IT); Laura Perin GALLANDT, Burbank, CA (US); Valentina VILLANI, Los Angeles, CA (US)

(73) Assignees: University of Southern California, Los Angeles, CA (US); Children's Hospital Los Angeles, Los Angeles, CA (US); Sapienza University of Rome, Rome (IT)

(21) Appl. No.: 19/372,964

(22) Filed: Oct. 29, 2025

Related U.S. Application Data
(60) Provisional application No. 63/713,473, filed on Oct. 29, 2024.

(51) Int. Cl. A23L 33/00; A23D 9/00; A23F 3/14; A23G 1/32; A23L 2/38; A23L 19/00; A23L 23/10; A23L 25/00; A61K 31/07; A61K 31/122; A61K 31/197; A61K 31/355; A61K 31/375; A61K 31/4188; A61K 31/4415; A61K 31/455; A61K 31/51; A61K 31/519; A61K 31/525; A61K 31/593; A61K 31/714; A61K 33/00; A61K 33/04; A61K 33/08; A61K 33/10; A61K 33/18; A61K 33/24; A61K 33/26; A61K 33/30; A61K 33/32; A61K 33/34; A61K 33/42; A61P 13/12

(52) U.S. Cl. CPC A23L 33/30; A23D 9/00; A23F 3/14; A23G 1/32; A23L 2/38; A23L 19/00; A23L 23/10; A23L 25/00; A61K 31/07; A61K 31/122; A61K 31/197; A61K 31/355; A61K 31/375; A61K 31/4188; A61K 31/4415; A61K 31/455; A61K 31/51; A61K 31/519; A61K 31/525; A61K 31/593; A61K 31/714; A61K 33/00; A61K 33/04; A61K 33/08; A61K 33/10; A61K 33/18; A61K 33/24; A61K 33/26; A61K 33/30; A61K 33/32; A61K 33/34; A61K 33/42; A61P 13/12

(57) ABSTRACT

A low-salt fasting-mimicking diet (LS-FMD) is provided for promoting kidney regeneration and treating kidney disease. The LS-FMD comprises cyclic dietary interventions that reduce caloric and sodium intake to induce fasting-like metabolic effects while maintaining essential nutrition. In animal models of puromycin-induced nephrosis, LS-FMD cycles restore normal proteinuria levels and improve nephron structure and function relative to untreated controls. The diet activates kidney-specific gene expression programs and promotes a quiescent, non-proliferative state in mature podocytes, supporting renal recovery. In human subjects with chronic kidney disease, administration of fasting-mimicking diet cycles for five days per month over three months reduces proteinuria and improves endothelial function. The invention demonstrates that kidney-specific fasting-mimicking dietary cycles have therapeutic potential for slowing or reversing progressive renal disorders.

### CROSS-REFERENCE TO RELATED APPLICATIONS

This application claims the benefit of U.S. provisional application Ser. No. 63/713,473 filed Oct. 29, 2024, the disclosure of which is hereby incorporated in its entirety by reference herein.

### STATEMENT REGARDING FEDERALLY SPONSORED RESEARCH OR DEVELOPMENT

This invention was made with government support under AG034906 awarded by the National Institutes of Health. The government has certain rights in the invention.

### TECHNICAL FIELD

In at least one aspect, the present invention is related to dietary methods for treating kidney damage and/or disease resulting from injury by progressive diseases (of the glomerulus and tubules) such as diabetic nephropathy, membranous nephropathy, focal segmental glomerulosclerosis, crescentic glomerulonephritis, membranoproliferative glomerulonephritis, mesangial proliferative glomerulonephritis, intraductal proliferative glomerulonephritis, mesangial capillary glomerulonephritis, ischemic nephropathy, glomerular disease based on systemic diseases, vascular disease, metabolic diseases, hereditary renal lesions (such as polycystic kidney disease, Alport Syndrome, etc.) or transplanted glomerular lesions. In addition, the disease can be a result of acute and non-acute (including trauma and chemical overdose).

### BACKGROUND

Chronic kidney disease (CKD) remains a major health challenge and a leading cause of premature morbidity and mortality (1-3). Loss of podocytes, the cells that together with glomerular endothelial cells (GECs) form the glomerular filtration barrier, are central culprits of most progressive CKD (4-7). Some pharmacological treatments can slow down CKD progression (8-11), but no intervention has proven to be effective in preventing podocyte damage and loss, and many cases of CKD progress to end-stage kidney disease.

Dietary interventions, including low protein diet, have been studied in patients with CKD (12-17). Recommendations regarding dietary intake have been proposed to minimize the risks of developing hypertension or diabetes, the major causes of CKD worldwide. A ketogenic diet was developed for patients with polycystic kidney disease (18, 19), showing promises in decreasing cyst formation. However, dietary interventions have had limited or no effects in promoting regeneration and reversing kidney damage and disease. Cycles of a diet that mimics the effects of fasting (FMD; a low-calorie, low-protein, low-glucose, high-fat diet) alternating with refeeding using a standard diet promote increased hematopoietic, intestinal, and neural stem cells and tissue regeneration in multiple systems including the pancreas and the immune system in mice (20-25). FMD cycles in humans reduce inflammation as well as markers and risk factors for age-related disease (25). Recently, FMD cycles were shown to have health-positive outcomes in patients with diabetic nephropathy (26).

Accordingly, there is a need for improved methods for treating renal disease.

### SUMMARY

In at least one aspect, a low-salt fasting-mimicking diet (LS-FMD) was created and evaluated as a modification of the original FMD to evaluate its impact on kidney damage. The diet was adjusted to reduce sodium, potassium, and phosphorus levels to align with the recommended daily intake for patients with chronic kidney disease (CKD), thereby creating an FMD regimen more suitable for mice, rats, and potentially for future applications in patients with kidney damage. (27-30).

In another aspect, cycles of a fasting-mimicking diet (FMD) have been shown to promote regeneration and reduce tissue damage in the pancreas, blood, gut, and nervous system of mice; however, their effects on kidney disease remain unknown. Moreover, the FMD has not previously been evaluated in rats. In the present disclosure, it is demonstrated that cycles of a newly developed low-salt fasting-mimicking diet (LS-FMD) restore normal proteinuria, nephron structure, and function in rats with puromycin-induced nephrosis over the long term. The LS-FMD induces the expression of nephrogenic markers that recapitulate renal developmental processes across multiple kidney structures and activates podocyte-lineage reprogramming pathways, while also promoting a quiescent state in mature podocytes. In a pilot randomized cross-over study involving patients with chronic kidney disease, FMD cycles promote indicators of renoprotection, including long-term reduction of proteinuria and improved endothelial function. These findings indicate that LS-FMD cycles, which reprogram multiple renal cell types and reverse glomerular damage, warrant further evaluation as a potential therapeutic approach for progressive kidney diseases.

In another aspect, data from a pilot randomized cross-over clinical trial applying three cycles of FMD in CKD patients indicate that this diet is well tolerated and causes a reduction of proteinuria maintained after 1 year of follow-up, an increase in circulating kidney progenitor-like cells, and amelioration of cardiac and endothelial damage markers, which are common within the CKD population.

Advantageously, LS-FMD-refeeding cycles result in long-term renoprotection by preventing podocyte loss and activating regenerative processes in different kidney compartments. Based on these results and the preliminary clinical data, this dietary regimen should be evaluated in larger clinical trials for its ability to slow down CKD progression and potentially avoid the need for dialysis and/or transplantation.

In another aspect, a method for treating kidney disease in a subject by administering a standard fasting-mimicking diet (FMD) is provided. As used herein, the term “standard FMD” refers to a fasting-mimicking diet that produces metabolic effects similar to fasting but is not restricted in sodium content. The normal FMD maintains sodium levels typical of standard nutritional formulations while retaining the defined caloric and macronutrient profiles that elicit fasting-like physiological responses.

In another aspect, a low-sodium fasting-mimicking diet composition is provided for use in treating or preventing a kidney disease in a human subject. The composition includes daily meal portions that are configured for a fasting-mimicking cycle of at least five days. When the daily meal portions for a given day are consumed, the composition provides less than about 2000 mg sodium per day, less than about 1500 mg potassium per day, and less than about 1500 mg phosphorus per day. The daily meal portions for day 1 provide about 900 to 1300 kilocalories with about 5 to 15 percent of calories from protein, 45 to 65 percent from fat, and 25 to 45 percent from carbohydrate. The daily meal portions for each of days 2 to 5 provide about 600 to 800 kilocalories per day with 5 to 15 percent of calories from protein, 35 to 55 percent from fat, and 40 to 55 percent from carbohydrate.

In another aspect, the composition provides no more than about 1500 mg sodium per day, no more than about 1000 mg potassium per day, and no more than about 1000 mg phosphorus per day.

In another aspect, the composition provides no more than about 1000 mg sodium per day, no more than about 500 mg potassium per day, and no more than about 500 mg phosphorus per day.

In another aspect, the sodium provided per day is between about 200 mg and about 1000 mg.

In another aspect, the fasting-mimicking cycle includes about 38 to 45 percent carbohydrate, about 8 to 12 percent protein, and about 45 to 55 percent fat.

In another aspect, the composition is packaged as a pre-portioned diet package that includes five separately packaged daily portions configured for sequential consumption during the fasting-mimicking cycle.

In another aspect, the pre-portioned diet package includes one or more of a nut-based nutrition bar, a cocoa-based nutrition bar, a first olive-containing composition, a kale-cracker composition, at least one vegetable soup, a first vegetable broth, a tea composition, an energy-drink composition, a micronutrient composition, and an algal-oil composition. The composition optionally further includes a mushroom soup, a tomato soup, a quinoa-minestrone, a bean-minestrone, a pumpkin soup, a second olive-containing composition, a second vegetable broth, a lemon-spearmint tea, and a hibiscus tea.

In another aspect, each soup, broth, tea, and energy-drink composition is provided as a mix or concentrate to be reconstituted with added water at the time of consumption.

In another aspect, the energy-drink composition includes glycerin and purified water as principal components, with glycerin present at about 20 to 60 weight percent and purified water present at about 40 to 80 weight percent.

In another aspect, the algal-oil composition includes Schizochytrium algal oil rich in docosahexaenoic acid, and the pre-portioned diet package provides an aggregate of about 600 mg docosahexaenoic acid over the fasting-mimicking cycle.

In another aspect, the micronutrient composition includes vitamins A, C, D3, E, K, B1, B2, B3, B5, B6, B7, B9, and B12, and minerals calcium, iron, magnesium, copper, manganese, selenium, chromium, molybdenum, zinc, and iodine.

In another aspect, each component of the composition is gluten-free or exceptionally low in gluten such that each component contains less than 20 ppm gluten or 20 to 100 ppm gluten, respectively. The serving-size ranges include nut bar 30 to 60 grams, cocoa bar 15 to 40 grams, each olive composition 10 to 20 grams, kale-cracker 20 to 60 grams, each soup 20 to 50 grams, first broth 5 to 15 grams, second broth 3 to 15 grams, and energy drink 1 to 5 ounces.

In another aspect, the day-specific nutrient constraints include for day 1 less than 30 grams sugar, less than 28 grams protein, 20 to 30 grams monounsaturated fat, and 6 to 10 grams polyunsaturated fat. For days 2 to 5 and any additional days, the constraints include less than 20 grams sugar, less than 18 grams protein, 10 to 15 grams monounsaturated fat, and 3 to 5 grams polyunsaturated fat.

In another aspect, the caloric intake for day 1 is 900 to 1300 kilocalories with 5 to 15 percent protein, 45 to 65 percent fat, and 25 to 45 percent carbohydrate, and the caloric intake for days 2 to 5 is 600 to 800 kilocalories with 5 to 15 percent protein, 35 to 55 percent fat, and 40 to 55 percent carbohydrate.

In another aspect, potassium is present at less than about 1500 mg per day, less than about 1000 mg per day, less than about 800 mg per day, less than about 500 mg per day, or less than about 300 mg per day.

In another aspect, phosphorus is present at less than about 1500 mg per day, less than about 1000 mg per day, less than about 800 mg per day, less than about 500 mg per day, or less than about 300 mg per day.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a further understanding of the nature, objects, and advantages of the present disclosure, reference should be had to the following detailed description, read in conjunction with the following drawings, wherein like reference numerals denote like elements and wherein:

FIGS. 1A, 1B, 1C, 1D, 1E, 1F, 1G, 1H, 1I, 1J, and 1K. Metabolic study in PAN rats. (A) H&E staining is shown of renal sections from a healthy rat and PAN rats at 10 days, 3 months, and 6 months after injection. Arrowheads indicate tubular damage and cast formation. Scale bars, 100 m. (B) ACR measurements in healthy rats (n=3 to 5) and PAN rats (n=4 to 10) are shown up to 6 months. Values are reported as median (ANOVA mixed effects analysis with Sidik's multiple comparison test). (C) Body weight is shown for the metabolic study on PAN rats fed ad libitum (n=4) or with LS-FMD (n=4) followed by 1 week of refeeding. Values are reported as means±SD (two-way ANOVA with Bonferroni test). Also shown from this study is (D) lean mass, (E) fat mass [all values in (D) and (E) are reported as median; two-way ANOVA with Tukey's test], (F) food intake (grams per hour), (G) cumulative food intake, (H) cumulative water intake, (I) respiratory exchange ratio (RER), (J) heat production, and (K) locomotor activity. All values in (F) through (K) are reported as means±SEM [two-way ANOVA with Sidik's multiple comparison test was performed in (I) and (J)]. Significant differences are indicated as *P<0.05, **P<0.01, ***P<0.001, and ****P<0.0001. hr, hours.

FIGS. 2A, 2B, 2C, 2D, 2E, and 2F. Multiple cycles of LS-FMD restore renal function and transcriptional landscape in PAN rats. (A) A timeline is shown outlining PAN induction and the application of multiple cycles of LS-FMD or FMD treatment. Each cycle of diet (blue boxes) is followed by refeeding with standard chow (white boxes), intermittently. Relevant assays are reported at specific time points. CTRL, control. (B) ACR and (C) BUN measurements are shown in PAN rats (n=3 to 5 per time point) and PAN rats fed with either FMD or LS-FMD (n=6 to 8 per time point) up to 6 weeks (day 131) after last cycle of diet. Healthy rats (n=3 to 5) had a baseline mean ACR=0.1. All values are reported as median (ANOVA mixed-effects analysis with Fisher's least significant difference and Tukey's tests, respectively). 4 w, 4 weeks; 6 w, 6 weeks. (D) ST integrated analysis of healthy rat (n=1), PAN rat ad libitum (n=1), and a PAN rat (n=1) with six cycles of LS-FMD (PAN/LS-FMD and euthanized after 6 weeks after the last cycle) identified nine clusters by unsupervised clustering as shown to the right of each spatial map. (E) UMAP of 7755 spots from the integration of a healthy (left, bottom quadrant), PAN (left, top quadrant), and PAN/LS-FMD (right, top quadrant) are shown. The right bottom quadrant shows the integration of all samples. Cluster annotation is reported on the side. (F) The graph displays the distribution of samples across identified clusters. Percentage of spots (x axis) for each cluster identified (y axis) in each sample (healthy, blue; PAN, orange; and PAN/LS-FMD, gray). Significant differences are indicated as *P<0.05 and **P<0.01.

FIGS. 3A, 3B, 3C, 3D, 3E, 3F, 3G, and 3H. LS-FMD protects glomeruli after PAN injury. (A) Spatial visualizations are shown of spots representing glomeruli from the integrated analysis of a healthy (light blue, 100), PAN (magenta, 24), and PAN/LS-FMD (blue, 100). (B) UMAPs highlight 224 glomerular spots between samples. (C) The table shows GO terms in PAN versus control (top) and PAN/LS-FMD versus PAN (bottom). (D) PAS staining showing glomerulosclerosis and matrix accumulation (asterisks) in PAN rats ad libitum versus PAN/LS-FMD, PAN/FMD, and healthy rats. Scale bars, 50 m. (E) WT1 immunostaining (red) and wheat germ agglutinin (WGA; green) of glomeruli from healthy, PAN ad libitum, and PAN/LS-FMD or PAN/FMD. Scale bars, 50 m. DAPI, 4′,6-diamidino-2-phenylindole. (F) Quantification of the glomerulosclerosis (GSI; n=3 to 5 per group, one-way ANOVA with Dunnett's test) and (G) glomerular area (n=70 to 180 glomeruli per sample, n=3 to 5 samples per group, one-way ANOVA with Tukey's test) based on PAS histological analysis. (H) The graph shows quantification of the average number of glomeruli (n gloms) from healthy, PAN ad libitum, or PAN/LS-FMD after six cycles (n=3 to 4 per group, one-way ANOVA with two-stage Benjamini, Krieger, and Yekutieli test). All values are reported as median. Significant differences are indicated as *P<0.05, **P<0.01, and ****P<0.0001.

FIGS. 4A, 4B, 4C, 4D, 4E, 4F, 4G, 4H, 4I, and 4J. LS-FMD induces up-regulation of stem and renal regenerative markers. (A) A timeline of the experimental procedures is shown. Healthy rats (n=9) and PAN rats (n=12) were fed with LS-FMD (one cycle), glomeruli were extracted, and gene expression was analyzed at day 14 after PAN, at the end of the diet, and at 24 and 72 hours after refeeding (n=3 per group). Healthy rats (n=6) fed ad libitum were included as control. (B) Heatmaps showing changes in gene expression of differentiation, of (C) stem cell/regenerative, and of (D) parietal epithelial cell (PEC) in PAN rats receiving LS-FMD. (E) Heatmaps showing changes in gene expression of differentiation, of (F) stem cell/regenerative, and of (G) PEC in healthy rats receiving LS-FMD. Scale bars report the levels of fold change of gene expression from lowest (blue) to highest (red) fold change in each heatmap. Healthy rats (n=3 in each experiment) were used for relative quantification [Student's t test of the replicate₂\hat{ }(−Delta CT) values for each gene in the control group and treatment groups]. Significant differences are indicated by asterisks. (H) A UMAP, split by cluster, is shown (top) of 5456 spots from the ST of a kidney from a healthy rat on a standard diet (blue) and a kidney from a healthy rat fed with one cycle of LS-FMD at 72 hours after refeeding (yellow). Each cluster reports its annotation. Cluster composition is reported in the bottom chart as percentage of sample distribution (x axis) per cluster (y axis). (I) UMAPs are shown from the ST of a kidney from a healthy rat on a standard diet (top right) and a kidney from a healthy rat fed with one cycle of LS-FMD at 72 hours after refeeding (top left). A merged view is also shown (bottom left). In the lower right quadrant, cluster 8 is highlighted which identifies glomerular spots (LS-FMD at 72 hours in blue and healthy in orange). (J) Tables of GO sets overrepresented in LS-FMD versus healthy based on up-regulated (top) and down-regulated genes (bottom). Significant values are indicated as *P<0.05, **P<0.01, and ***P<0.001.

FIGS. 5A, 5B, 5C, 5D, 5Ea, 5Eb, 5Ec, 5Ed, 5F, 5G, 5H, and 5I. LS-FMD shows transcriptional signature of protection for damaged podocytes. (A) Shown is the integrated UMAP of single nuclei from kidneys of healthy rats (n=4), PAN rats fed ad libitum at day 14 (n=3), PAN rats with one cycle of LS-FMD at the end of diet (EoD; n=3), 24 hours after refeeding (n=3), and 72 hours after refeeding (n=3). A total of 54,757 nuclei were analyzed, and 15 cell types were identified. n=123 nuclei remained unclassified. (B) The heatmap shows gene expression of renal cell populations classified in (A). (C) UMAP (top) of re-clustered podocytes (451 nuclei) from (A) showing two clusters: 1 (blue) and 2 (magenta). The table shows ratio of podocyte nuclei/total number of nuclei (POD N/TOT N) in each group and relative percentage. (D) Pie charts representing experimental group's composition of clusters 1 and 2: control (light blue), PAN (magenta), EoD (orange), 24 hours of refeeding (24-hr RF; teal), and 72 hours of refeeding (72-hr RF; purple). Enriched GO per each condition versus all other conditions for both clusters are reported as side tables (color coded). (E) Trajectory inference analysis (top) of podocytes showing co-expression of Pax8 (blue), CD24 (green), Aldhlal (red), and Nphsl (expression shown as dot size). The overlay of clusters 1 and 2 of podocytes along the trajectory is shown in the bottom left quadrant, and the pseudo-time ordering of nuclei, starting from the right side identified as the root, is shown in the bottom right quadrant. (F) Staining for PAX8 (red) and NEPHRIN (orange) expression in glomeruli of rats at EoD. Nuclei are stained in DAPI. Scale bar, 50 m. Magnified sections show co-expression of PAX8 and NEPHRIN (asterisks). Cells positive for NEPHRIN but negative for PAX8 are also indicated (arrowheads). (G) Graph showing the distribution of podocytes [NEPHRIN+cells (N+)], at different cell cycle phases, isolated from POD-FUCCI-Alport mice (5 months old) and subjected to one cycle of LS-FMD, measured by flow cytometry analysis. All values are reported as median (n=3 to 4 per group, two-way ANOVA with Tukey's test). AS, Alport syndrome. (H) UMAP of re-clustered glomerular endothelial cells (GECs; left), showing a total of 216 nuclei. Right panel shows bar chart with clusters' composition by different experimental groups: control (light blue), PAN (magenta), EoD (orange), and 24 hours (teal). No GEC nuclei were identified in the 72-hour group. (I) UMAP of mesangial cells (left), showing a total of 1135 nuclei. Right panel shows bar chart with clusters' composition by different experimental groups: control (light blue), PAN (magenta), EoD (orange), 24 hours (teal), and 72 hours (purple). Significant differences are indicated as *P<0.05 and **P<0.01.

FIGS. 6A, 6B, 6C, 6D, 6E, 6F, 6G, 6H, 6I, 6J, 6K, and 6L. FMD ameliorates metabolic profile in patients with CKD. Patients with stage III CKD (n=13) were subjected to three cycles of the human version of the FMD, and the following parameters were measured before and after treatment: (A) body weight, (B) lean mass (BIA analysis), (C) fat mass (BIA analysis), and (D) body mass index. (E) Percentage of muscle mass is shown and was calculated using the following formula: muscle mass/(lean mass+fat mass)*100. Also shown from this study are (F) hand grip strength, (G) insulinemia, (H) glycemia, (I) total cholesterol, (J) low-density lipoprotein, (K) high-density lipoprotein, and (L) triglycerides. All values are reported as median (paired t test). Significant differences are indicated as *P<0.05, **P<0.01, and ***P<0.001.

FIGS. 7A, 7B, 7C, 7D, 7E, 7F, 7G, 7H, 7I, 7J, 7K, 7L, 7M, and 7N. FID ameliorates renal dysfunction and reduces endothelial dysfunction in patients with CKD. Renal and endothelial functions were assessed in patients with stage III CKD (n=13) undergoing three FMD cycles: (A) 24-hour proteinuria is shown before and after FMD. (B) Serum creatinine is shown before and after FMD. (C) Creatininuria is shown before and after FMD. (D) Proteinuria to creatininuria ratio is shown before and after FMD. (E) eGFR is shown before and after FMD. (F) Circulating IGF-1 is shown before and after FMD. (G) Circulating C-reactive protein is shown before and after FMD. (H) Epicardial fat is shown before and after FMD. (I) Flow-mediated dilation of the brachial artery is shown before and after FMD. All values in (A) through (I) are reported as median (paired t test). (J) Assessment at 1 year of follow-up on n=10 patients including C-reactive protein (one-way ANOVA with Tukey's test), (K) proteinuria (one-way ANOVA with two-stage Benjamini, Krieger, and Yekutieli test), (L) estimation plot of proteinuria progression, (M) proteinuria in patients with initial proteinuria greater than 1 g/24 hours (before the beginning of the FMD treatment, one-way ANOVA with two-stage Benjamini, Krieger, and Yekutieli test). All values in (J), (K), and (M) are reported as means±SD. (N) Shown is the percentage of circulating progenitor-like cells (CD24+CD45−CD133+) found in patients' sera. Values are reported as median. Significant differences are indicated as *P<0.05, **P<0.01, ***P<0.001, and ****P<0.0001.

### DETAILED DESCRIPTION

Reference will now be made in detail to presently preferred embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

When referring to a numeral quantity, in a refinement, the term “less than” includes a lower non-included limit that is 5 percent of the number indicated after “less than” truncated or rounded to 2 significant figures. For example, “less than 20” includes a lower non-included limit of 1 in a refinement. Therefore, this refinement of “less than 20” includes a range between 1 and 20. In another refinement, the term “less than” includes a lower non-included limit that is, in increasing order of preference, 20 percent, 10 percent, 5 percent, or 1 percent of the number indicated after “less than” truncated or rounded to 2 significant figures.

Unless stated to the contrary, weight percentages for an ingredient of a fasting-mimicking diet package component are referenced to the total weight of that component.

Diet compositions described for a fifth day of the FMD and any packages thereof can also be provided for any additional days if the FMD is extended beyond 5 days.

In numerical ranges having 0 as a lower limit, a refinement can have an alternative lower limit that is 5 percent of the upper limit truncated or rounded to 2 significant figures. For example, “0 to 20” includes a lower non-included limit of 1 in a refinement. Therefore, this refinement of “0 to 20” includes a range from 1 to 20. In another refinement, numerical ranges having 0 as a lower limit includes a lower limit that is, in increasing order of preference, 20 percent, 10 percent, 5 percent, or 1 percent of the upper limit truncated or rounded to 2 significant figures.

Throughout this application, where publications are referenced, the disclosures of these publications in their entireties are hereby incorporated by reference into this application to more fully describe the state of the art to which this invention pertains.

The term “subject” refers to a human or other animal, including birds and fish as well as all mammals such as primates (particularly higher primates), horses, birds, fish sheep, dogs, rodents, guinea pigs, pig, cat, rabbits, and cows.

### Abbreviations

“ACE” means Angiotensin-Converting Enzyme.
“ACR” means Albumin-to-Creatinine Ratio.
“ANOVA” means Analysis of Variance.
“AS” means Alport Syndrome.
“BMI” means Body Mass Index.
“BUN” means Blood Urea Nitrogen.
“CHLA” means Children's Hospital Los Angeles.
“CKD” means Chronic Kidney Disease.
“DNA” means Deoxyribonucleic Acid.
“DAPI” means 4′,6-Diamidino-2-Phenylindole.
“EoD” means End of Diet.
“eGFR” means Estimated Glomerular Filtration Rate.
“ESRD” means end stage renal disease.
“FMD” means Fasting-Mimicking Diet.
“FUCCI” means Fluorescent Ubiquitination-based Cell Cycle Indicator.
“GEC” means Glomerular Endothelial Cell.
“GO” means Gene Ontology.
“GSI” means Glomerulosclerosis Index.
“H&E” means Hematoxylin and Eosin.
“IACUC” means Institutional Animal Care and Use Committee.
“IGF-1” means Insulin-like Growth Factor 1.
“IGFBP-1” means Insulin-like Growth Factor Binding Protein 1.
“LOH” means Loop of Henle.
“LS-FMD” means Low-Sodium Fasting-Mimicking Diet.
“MRL/lpr” refers to a strain of mice used in lupus nephritis models.
“PAS” means Periodic Acid-Schiff.
“PEC” means Parietal Epithelial Cell.
“PCR” means Polymerase Chain Reaction.
“PT” means Proximal Tubule.
“RER” means Respiratory Exchange Ratio.
“SEM” means Standard Error of the Mean.
“snRNA-seq” means Single Nucleus RNA Sequencing.
“ST” means Spatial Transcriptomics.
“UMAP” means Uniform Manifold Approximation and Projection.
“WB” means Western Blot.
“WT1” means Wilms Tumor 1 Protein.

The term “subject” refers to a human or animal, including all mammals such as primates (particularly higher primates), sheep, dog, rodents (e.g., mouse, or rat), guinea pig, goat, pig, cat, rabbit, and cow.

The term “fasting-mimicking diet” (FMD) means a diet that mimics the effects of fasting typically by providing a subject with at most 50% of their normal caloric intake but with some nutritional component so that fasting is mimicked while a subject is nourished and not completely starved. Examples of useful fasting-mimicking and enhancing diets and methods for monitoring the effects of these diets on markers such as IGF-1 and IGFBP1 in the context of the present invention are set forth in U.S. patent application Ser. No. 14/273,946 filed May 9, 2014; Ser. No. 14/497,752 filed Sep. 26, 2014; Ser. No. 12/910,508 filed Oct. 22, 2010; Ser. No. 13/643,673 filed Oct. 26, 2012; Ser. No. 13/982,307 filed Jul. 29, 2013; Ser. No. 14/060,494 filed Oct. 22, 2013; Ser. No. 14/178,953 filed Feb. 12, 2014; Ser. No. 14/320,996 filed Jul. 1, 2014; Ser. No. 14/671,622 filed Mar. 27, 2015; the entire disclosure of these patent applications is hereby incorporated by reference. The fasting-mimicking diet set forth in U.S. patent application Ser. Nos. 14/060,494 and 14/178,953 are found to be particularly useful in the present invention. Additional examples of FMD diets are found in U.S. patent application Ser. No. 15/148,251 and WIPO Pub. No. WO2011/050302 and WIPO Pub. No. WO2011/050302; the entire disclosures of which are hereby incorporated by reference. In a refinement, the low sodium FMD used herein can be constructed by limiting the amount of sodium (i.e., sodium chloride) in these prior FMDs. In a refinement, the FMD is a ketogenic FMD. Similarly, the low sodium FMD can be a low sodium ketogenic FMD. Therefore, in the descriptions set forth herein, the term ketogenic FMD can be replaced by FMD and vice versa as needed. Similarly, in the descriptions set forth herein, the term ketogenic fasting-mimicking diet can be replaced by fasting-mimicking diet and vice versa as needed. Finally, the term low sodium FMD can be replaced by low sodium ketogenic FMD and vice versa.

In at least one aspect, the present invention provides a method for treating kidney disease in a subject by administering cycles of a low-salt fasting-mimicking diet to the subject, wherein each cycle comprises at least five days of restricted caloric intake followed by a refeeding period. The low-salt fasting-mimicking diet provides less than 2000 mg of sodium per day of the FMD, 1500 mg of potassium per day of the FMD, and less than 1500 mg of phosphorus per day of the FMD. The diet is designed as a plant-based regimen to provide macro- and micronutrients, including essential minerals, vitamins, and fatty acids, to achieve fasting-like effects on serum IGF-1, IGFBP-1, glucose, and ketone bodies while minimizing adverse effects typically associated with fasting. In certain refinements, the diet provides less than about 1500 mg of sodium per day of the FMD, 1000 mg of potassium per day of the FMD, and less than 1000 mg of phosphorus per day of the FMD. In additional refinements, the diet provides less than about 1000 mg of sodium per day of the FMD, 500 mg of potassium per day of the FMD, and less than 500 mg of phosphorus per day of the FMD. In some refinements, the diet provides less than, in increasing order of preference 3000 mg of sodium per day of the FMD, 2000 mg of sodium per day of the FMD, 1500 mg of sodium per day of the FMD, 1000 mg of sodium per day of the FMD, or 500 mg of sodium per day of the FMD and at least 0 mg of sodium per day of the FMD, 10 mg of sodium per day of the FMD, 100 mg of sodium per day of the FMD, 150 mg of sodium per day of the FMD, or 200 mg of sodium per day of the FMD. In some refinements, the diet provides less than, in increasing order of preference 2500 mg of potassium per day of the FMD, 2000 mg of potassium per day of the FMD, 1500 mg of potassium per day of the FMD, 1000 mg of potassium per day of the FMD, 800 mg of potassium per day of the FMD, 500 mg of potassium per day of the FMD, or 300 mg of potassium per day of the FMD and at least 0 mg of potassium per day of the FMD, 10 mg of potassium per day of the FMD, 100 mg of potassium per day of the FMD, 150 mg of potassium per day of the FMD, or 200 mg of potassium per day of the FMD. In some refinements, the diet provides less than, in increasing order of preference 2500 mg of phosphorus per day of the FMD, 2000 mg of phosphorus per day of the FMD, 1500 mg of phosphorus per day of the FMD, 1000 mg of phosphorus per day of the FMD, 800 mg of phosphorus per day of the FMD, 500 mg of phosphorus per day of the FMD, or 300 mg of phosphorus per day of the FMD and at least 0 mg of phosphorus per day of the FMD, 10 mg of phosphorus per day of the FMD, 100 mg of phosphorus per day of the FMD, 150 mg of phosphorus per day of the FMD, or 200 mg of phosphorus per day of the FMD. In some refinements, the diet provides at least 10 mg, 50 mg, 100 mg, or 200 mg phosphorus per day of the FMD, at least 10 mg, 100 mg, 200 mg, or 400 mg potassium per day of the FMD, and at least 10 mg, 50 mg, 100 mg, or 200 mg sodium per day of the FMD.

In another aspect, the low-salt fasting-mimicking diet includes specific sources for each essential electrolyte. Sodium is available as salts, including sodium chloride (NaCl), and potassium can be supplied as salts such as potassium iodide (KI). Natural plant foods are typically low in sodium, which supports the “low-salt” FMD design. Minimal sodium may be provided from trace sources or small additions of natural sea salt or mineralized broth to reach a safe minimum (˜200 mg/day). Potassium is plentiful in most plant foods. In an FMD designed for kidney protection, you can maintain a restricted but adequate intake (≈400-1000 mg/day) using limited portions of vegetables, fruits, and grains without supplementation. Phosphorus is widely distributed in foods, especially those rich in protein or phosphate additives. Major natural sources include meats, poultry, fish, eggs, and dairy products, which provide highly absorbable phosphorus, while plant-based foods such as beans, lentils, nuts, seeds, and whole grains contain phosphorus mostly in the less bioavailable phytate form. Fruits and vegetables contribute only small amounts. Processed foods, such as cola drinks, fast foods, and packaged meats or cheeses, often contain phosphate additives that are nearly 100% absorbed. For kidney-protective or fasting-mimicking diets, it is preferable to rely on plant-based sources and avoid animal proteins and additive-rich processed foods to reduce phosphorus load and prevent hyperphosphatemia. In a refinement, the phosphorus in the FMD can be derived from phosphate-based compounds. This composition ensures electrolyte levels align with the requirements for kidney health in chronic kidney disease models.

In another aspect, the low-salt fasting-mimicking diet modulates kidney cell regeneration, promotes a quiescent state in podocytes, and reduces glomerular sclerosis. The diet may be administered for three to six cycles or more, with each cycle separated by a refeeding period of at least seven days. In certain refinements, the low-salt fasting-mimicking diet provides a daily caloric intake for day one from 900 to 1300 kcal, with 5-15% of calories from protein, 45-65% from fat, and 25-45% from carbohydrates. For days two to five, the daily caloric intake ranges from 600 to 800 kcal, with 5-15% of calories from protein, 35-55% of calories from fat, and 40-55% of calories from carbohydrates. In further refinements, the daily caloric intake for day one is from 1000 to 1200 kcal, with 8-12% of calories from protein, 50-60% of calories from fat, and 30-40% of calories from carbohydrates, while days two to five provide 700 to 750 kcal per day, with 7-11% of calories from protein, 40-50% of calories from fat, and 45-50% of calories from carbohydrates.

In another aspect, the low-salt fasting-mimicking diet provides a daily caloric intake for day one of the fasting-mimicking cycle that is less than, in increasing order of preference, 1500 kcal, 1400 kcal, 1300 kcal, 1200 kcal, or 1100 kcal, and at least, in increasing order of preference, 600 kcal, 700 kcal, 800 kcal, 900 kcal, or 1000 kcal. In certain refinements, the day-one macronutrient distribution comprises, in increasing order of preference, 5%, 6%, 8%, 10%, 12%, or 15% of total calories from protein; 45%, 50%, 55%, 60%, or 65% from fat; and 25%, 30%, 35%, 40%, or 45% from carbohydrates. In further refinements, the daily caloric intake for days two through five of the fasting-mimicking cycle is less than, in increasing order of preference, 900 kcal, 850 kcal, 800 kcal, 750 kcal, or 700 kcal, and at least, in increasing order of preference, 400 kcal, 500 kcal, 600 kcal, 650 kcal, or 700 kcal. The macronutrient composition for days two through five provides, in increasing order of preference, 5%, 6%, 8%, 10%, 12%, or 15% of calories from protein; 35%, 40%, 45%, 50%, or 55% from fat; and 40%, 45%, 50%, or 55% from carbohydrates.

In another aspect, the diet may include a range of individually boxed food items, such as vegetable-based soups, energy bars, energy drinks, chip snacks, and tea, along with a dietary supplement formulated to provide high levels of minerals, vitamins, and essential fatty acids, enabling subjects to choose consumption times while avoiding unintended intake of components designated for different days.

In another aspect, the low-salt fasting-mimicking diet induces metabolic changes in kidney cells, promoting gene expression associated with lipid metabolism, biological oxidation, and gluconeogenesis. Additionally, the diet modulates the expression of nephrogenic gene programs to reprogram renal cell types, including podocytes, glomerular endothelial cells, and proximal tubular cells.

In another aspect, the low-salt fasting-mimicking diet modulates the cell cycle by inducing increase in modulators that inhibit cell cycle progression thereby promoting maintenance of podocytes in their quiescent and highly differentiated state.

In another aspect, the method may be used to treat chronic kidney disease (CKD). In a refinement, the method reduces proteinuria and blood urea nitrogen (BUN) levels in CKD patients. In a refinement, the low-salt fasting-mimicking diet provides sustained improvements in CKD for up to one year post-treatment. Advantageously, the low-salt fasting-mimicking diet reduces markers of inflammation and improves endothelial function in CKD patients. Furthermore, in another refinement, the low-salt fasting-mimicking diet may promote an increase in circulating kidney progenitor cells characterized by CD24⁺CD133⁺CD45⁻ markers.

In an embodiment, a method for treating kidney damage, and in particular, ESRD is provided. The method includes a step of identifying a subject having kidney damage and/or kidney disease, including but not limited to ESRD and/or disease resulting from injury of progressive disease (of the glomerulus and tubules) such as diabetic nephropathy, membranous nephropathy, focal segmental glomerulosclerosis, crescentic glomerulonephritis, membranoproliferative glomerulonephritis, mesangial proliferative glomerulonephritis, intraductal proliferative glomerulonephritis, mesangial capillary glomerulonephritis, ischemic nephropathy, glomerular disease based on systemic diseases, vascular disease, metabolic diseases, hereditary renal lesions (such as polycystic kidney disease, Alport Syndrome, etc.) or transplanted glomerular lesions. In addition, the disease can be a result of acute and non-acute (including trauma and chemical overdose). Methods for identifying kidney damage include, but are not limited to, blood tests (e.g., glomerular filtration rate), urine tests, imaging studies (e.g., MRI, CAT scans, IVP, and the like), and kidney biopsies. The subject is then administered a low sodium FMD (LS-FMD) for a first time period. In a refinement, the first time period is 1 to 10 days (i.e., 1, 2, 3, 4, 5, 6, 7, 8, 9, or 10 days). Typically, the first time period is 5 or 6 days. In a further variation, the low sodium FMD is repeating at predefined intervals. Such intervals can be from 5 to 26 days or longer. The total duration for treatment (i.e., the treatment time period) can be from a month to several years (e.g., 1, 2, 3, 4, or 5 years) during which the low sodium FMD is periodically or repeatedly administered. During and/or after treatment the subject's kidney disease can be re-evaluated using the methods for identifying kidney damage as set forth above. It should be appreciated that although low sodium ketogenic FMD diets are optimal for treating kidney disease, ketogenic FMD's that are not low sodium can also provide a benefit for treating kidney disease. Therefore, a normal ketogenic FMD (i.e., not low sodium) can be substituted in the descriptions of the low sodium FMD and vice versa as needed. In this context, “not low sodium” means that that the diet is not sodium restrict. For example, the FMD can include greater than 2000 mg of sodium per day and/or greater than 1500 mg of potassium per day, and greater than 1500 mg of phosphorus per day.

Advantageously, the low sodium FMD decreases proteinuria and BUN and ameliorates glomerular histology and stabilizes eGFR. The low sodium FMD re-establishes normal value of eGFR in urine. Average estimated GFR in different age groups is 116 mL/min/1.73 m² for ages 20-29 years, 107 mL/min/1.73 m² for ages 30-39 years, 99 mL/min/1.73 m for ages 40-49 years, 93 mL/min/1.73 m for ages 50-59 year, 85 mL/min/1.73 m for ages 60-69 year, and 75 mL/min/1.73 m for ages 70 years or older. In a refinement, cycles of the low sodium FMD are repeated a plurality of times until the EGFR are within a predetermined percent (e.g., 50%, 40%, 30%, or 20%) of the age-appropriate normal value for the subject being treated. Normal ranges for BUN are Adults up to 60 years of age: 6-20 mg/dL for adults 18 to 60 years of age and: 8-23 mg/dL for adults over 60 years of age. In a refinement, cycles of the low sodium FMD are repeated a plurality of times until the BUN are within a predetermined percent (e.g., 50%, 40%, 30%, or 20%) of the appropriate upper or lower normal ranges for the subject being treated. In another refinement, cycles of the low sodium FMD are repeated a plurality of times until the albumin concentrations in a subject's urine are less than a predetermined amount (e.g., less than, in increasing order of preference, 300 mg/dL, 100 mg/dL, or 30 mg/dL.) The amount of albumin in urine can be determined by Microalbumin Test or by the protein dipstick test.

In some variations, a refeeding diet is administered to the subject for a second time period. The diet provides an overall calorie consumption that is within 10 percent of a subject's normal calorie consumption. In this context, normal calorie consumption is the number of kilocalories needed to maintain the subject's weight at their pretreatment weight (e.g., the day before treatment is commenced) or to maintain a predetermined target weight. Therefore, the refeeding diet can be referred to as a “refeeding diet.” Although the present invention is not significantly limited by the second time period, the second time period can be from 7 days to 6 months or longer. Typically, the second time period is from 5 days to 26 days or longer following the low sodium diet. In some refinements, the refeeding diet provides at most, in increasing order of preference, 2500 kcal/day, 2400 kcal/day, 2300 kcal/day, 2200 kcal/day, 2100 kcal/day, 2000 kcal/day, 1900 kcal/day, 1800 kcal/day, 1700 kcal/day, 1600 kcal/day, or 1500 kcal/day. In some further refinements, the diet provides at least, in increasing order of preference, 1200 kcal/day, 1300 kcal/day, 1400 kcal/day, 1500 kcal/day, 1600 kcal/day, 1700 kcal/day, or 1800 kcal/day. In a refinement, the low sodium FMD is administered biweekly (i.e., every two weeks) after kidney damage has been identified. Advantageously, this new low sodium FMD increases circulating mesenchymal stem cells and improves kidney function.

Typically, the low sodium FMD is based on human foods that allow nourishment during periods of low-calorie consumption and high fat. In a variation, the low sodium FMD provides less than about 1000 mg per day of sodium. In a variation, the low sodium FMD provides sodium (e.g., sodium chloride) in an amount that less than or equal to, in increasing order of preference, 1000 mg per day, 900 mg per day, 800 mg per day, 700 mg per day, 600 mg per day, or 500 mg per day. In a refinement, the low sodium FMD provides sodium (e.g., sodium chloride) in an amount that is greater than or equal to, in increasing order of preference, 200 mg per day, 100 mg per day, 50 mg per day, 10 mg per day, or 0 mg per day. The low sodium FMD typically includes kilocalories from carbohydrates in an amount from about 30 to 70 percent of the total kilocalories of the FMD, kilocalories from protein in an amount less than about 10 percent of the total kilocalories of the FMD, and kilocalories from fat carbohydrates in an amount from about 30 to 70 percent of the total kilocalories of the FMD. In a refinement, the low sodium FMD typically includes kilocalories from carbohydrates in an amount from about 35 to 50 percent of the total kilocalories of the FMD, kilocalories from protein in an amount from about 0.5 to 10 percent of the total kilocalories of the FMD, and kilocalories from fat carbohydrates in an amount from about 40 to 60 percent of the total kilocalories of the FMD. In a further refinement, the source of kilocalories is 42% from carbohydrates, 2% protein, and 47% fat. In some refinements, the diet is mainly composed of olive oil and vegetable soups.

In a variation, the low sodium FMD diet provides less than 30 g of sugar on day 1 and less than 20 g of sugar on days 2-5 and any additional days of the diet. The diet should contain less than 28 g of proteins on day 1 and less than 18 g of proteins on days 2-5 and any additional days (e.g., days 6-10) of the diet, mostly or completely from plant-based sources. The diet should contain between 20 and 30 grams of monounsaturated fats on day 1 and 10-15 grams of monounsaturated fats on days 2-5 and any additional days (e.g., days 6-10) of the diet. The diet should contain between 6 and 10 grams of polyunsaturated fats on day 1 and 3-5 grams of polyunsaturated fats on days 2-5 and any additional days (e.g., days 6-10) of the diet. The diet should contain less than 12 g of saturated fats on day 1 and less than 6 grams of saturated fats on days 2-5 and any additional days (e.g., days 6-10) of the diet. Typically, the fats on all days are derived from a combination of the following: Almonds, Macadamia Nuts, Pecans, Coconut, Coconut oil, Olive Oil, and Flaxseed. In a refinement, the FMD diet includes over 50% of the recommended daily value of dietary fiber on all days. In a further refinement, the amount of dietary fiber is greater than 15 grams per day on all five days. The diet should contain 12-25 grams of glycerol per day on days 1-5 (or 2-5) and any additional days (e.g., days 6-10) of the diet. In a refinement, glycerol is provided at 0.1 grams per pound body weight/day. Characteristically, each of the components of the low sodium FMD are chosen to contain or prepared with components having low amounts of sodium (i.e., sodium chloride). For some components (e.g., vegetables), the components can be washed with water (to extract the sodium) such that the sodium chloride is removed.

In a variation, the low sodium FMD includes the following micronutrients (at least 95% non-animal based): over 5,000 IU of vitamin A per day (days 1-5 and any additional days); 60-240 mg of vitamin C per day (days 1-5); 400-800 mg of calcium per day (days 1-5 and any additional days); 7.2-14.4 mg of iron per day (days 1-5 and any additional days); 200-400 mg of magnesium per day (days 1-5 and any additional days); 1-2 mg of copper per day (days 1-5 and any additional days); 1-2 mg of manganese per day (days 1-5 and any additional days); 3.5-7 mcg of selenium per day (days 1-5 and any additional days); 2-4 mg of Vitamin B1 per day (days 1-5 and any additional days); 2-4 mg of Vitamin B2 per day (days 1-5 and any additional days); 20-30 mg of Vitamin B3 per day (days 1-5 and any additional days); 1-1.5 mg of Vitamin B5 per day (days 1-5 and any additional days); 2-4 mg of Vitamin B6 per day (days 1-5 and any additional days); 240-480 mcg of Vitamin B9 per day (days 1-5 and any additional days); 600-1000 IU of Vitamin D per day (days 1-5 and any additional days); 14-30 mg of Vitamin E per day (days 1-5 and any additional days); over 80 mcg of Vitamin K per day (days 1-5 and any additional days); 16-25 mcg Vitamin B12 are provided during the entire low sodium FMD; and docosahexaenoic acid (DHA, algae-derived) provided over the first five day and/or any additional days. For example, 600 mg can be given over the first 5 days of the FMD. The FMD diet provides high micronutrient content mostly (i.e., greater than 50 percent by weight) from natural sources such as kale, cashews, yellow bell pepper, onion, lemon juice, yeast, turmeric, mushroom, carrot, olive oil, beet juice, spinach, tomato, collard, nettle, thyme, salt, pepper, Vitamin B12 (Cyanocobalamin), beets, butternut squash, oregano, tomato juice, orange juice, celery, romaine lettuce, cumin, orange rind, citric acid, nutmeg, cloves, and combinations thereof. Table 1 provides an example of additional micronutrient supplementation that can be provided in the FMD diet:

TABLE 1
Micronutrient Supplementation

| | Supplement | Formula | Amount | Amount Range | Unit |
|---|---|---|---|---|---|
| Vit A | | | 1250 IU | 900-1600 | IU |
| Vit C | Ascorbic Acid | C₆H₈O₆ | 15.0000 | 10-20 | mg |
| Ca | Calcium | CaCO₃ | 80.0000 | 60-100 | mg |
| | Carbonate | | | | |
| Fe | Ferrous Fumarate | C₄H₂FeO₄ | 4.5000 | 3-6 | mg |
| Vit D3 | Cholecalciferol | C₂₇H₄₄O | 0.0025 | 0.001-0.005 | mg |
| Vit E | dl-Alpha | C₂₉H₅₀O₂ | 5.0000 | 3-7 | mg |
| | Tocopherol | | | | |
| | Acetate | | | | |
| Vit K | Phytonadione | | 0.0200 | 0.1-0.04 | mg |
| Vit B1 | Thiamine | C₁₂H₁₇N₅O₄S | 0.3750 | 0.15-0.5 | mg |
| | Mononitrate | | | | |
| Vit B2 | Riboflavin E101 | C₁₇H₂₀N₄O₆ | 0.4250 | 0.2-0.6 | mg |
| Vit B3 | Niacinamide | C₆H₆N₂O | 5.0000 | 3-7 | mg |
| Vit B5 | Calcium | C₁₈H₃₂CaN₂O₁₀ | 2.5000 | 1.5-4.0 | mg |
| | Pantothenate | | | | |
| Vit B6 | Pyridoxine | C₈H₁₁NO₃•HCl | 0.5000 | 0.3-0.7 | mg |
| | Hydrochloride | | | | |
| Vit B7 | Biotin | C₁₀H₁₆N₂O₃S | 0.0150 | 0.01-0.02 | mg |
| Vit B9 | Folic Acid | C₁₉H₁₉N₇O₆ | 0.1000 | 0.07-0.14 | mg |
| Vit | Cyanocobalamin | C₆₃H₈₈CoN₁₄O₁₄P | 0.0015 | 0.001-0.002 | mg |
| B12 | | | | | |
| Cr | Chromium | Cr(C6H4NO2)3 | 0.0174 | 0.014-0.022 | mg |
| | Picolinate | | | | |
| Cu | Cupric Sulfate | CuSO4 | 0.2500 | 0.18-0.32 | mg |
| I | Potassium Iodide | KI | 0.0375 | 0.03-0.045 | mg |
| Mg | Magnesium | MgO | 26.0000 | 20-32 | mg |
| | Oxide | | | | |
| Mn | Manganese | MnSO₄ | 0.5000 | 0.3-0.7 | mg |
| | Sulfate | | | | |
| Mo | Sodium | Na₂MoO₄ | 0.0188 | 0.014-0.023 | mg |
| | Molybdate | | | | |
| Se | Sodium Selenate | Na₂O₄Se | 0.0175 | 0.014-0.023 | mg |
| Zn | Zinc Oxide | ZnO | 3.7500 | 3-5 | mg |

In another embodiment, a diet package for implementing the low-sodium fasting-mimicking diets set forth above is provided. In general, the diet package includes rations for implementing the fasting-mimicking diets set forth above. The sources of these rations are also set forth above. In particular, the rations are divided into portions for each cycle of the low-sodium fasting-mimicking diet. In a variation, the diet package includes rations having (i.e., providing) kilocalories from carbohydrates in an amount from about 30 to 70 percent of the total kilocalories of the FMD, kilocalories from protein in an amount less than about 10 percent (e.g., 0.1 to 10 percent protein of the total kilocalories of the FMD, and kilocalories from fat carbohydrates in an amount from about 30 to 70 percent of the total kilocalories of the FMD. In a refinement, the diet package includes rations having kilocalories from carbohydrates in an amount from about 35 to 50 percent of the total kilocalories of the FMD, kilocalories from protein in an amount from about 0.5 to 10 percent of the total kilocalories of the FMD, and kilocalories from fat carbohydrates in an amount from about 40 to 60 percent of the total kilocalories of the FMD. In a further refinement, the diet package includes rations having kilocalories is 42% from carbohydrates, 2% protein, and 47% fat. In some refinements, the diet is mainly composed of olive oil and vegetable soups. In a refinement, the rations are prepared such that the low sodium FMD provides sodium (e.g., sodium chloride) in an amount that less than or equal to, in increasing order of preference, 1000 mg per day, 900 mg per day, 800 mg per day, 700 mg per day, 600 mg per day, or 500 mg per day.

Alternatively, the rations are divided into portions for each day of the fasting-mimicking diet. In another variation, the rations are divided into portions for each meal of the low sodium fasting-mimicking diet. For example, the diet package includes a first set of rations for a low sodium fasting-mimicking diet to be administered for the first time period to a subject, the low sodium fasting-mimicking diet providing from 4.5 to 7 kilocalories per pound of subject for a first day and 3 to 5 kilocalories per pound of subject per day for a second to fifth day (and any additional days) of the low sodium FMD. In a refinement, the low sodium FMD is a low protein diet. In a variation, the diet package includes rations that provide less than 30 g of sugar on the first day; less than 20 g of sugar on the second to fifth days; less than 28 g of proteins on the first day; less than 18 g of proteins on the second to fifth days; 20 to 30 grams of monounsaturated fats on the first day; 10 to 15 grams of monounsaturated fats on the second to fifth days; between 6 and 10 grams of polyunsaturated fats on the first day; 3 to 5 grams of polyunsaturated fats on the second to fifth days; less than 12 g of saturated fats on the first day; less than 6 grams of saturated fats on the second to fifth days; and 12 to 25 grams of glycerol per day on the second to fifth days. In a refinement, the diet package further includes sufficient rations to provide the micronutrients set forth above. In a further refinement, the diet package provides instructions providing details of the methods set forth above. In a refinement, the rations are prepared such that the low sodium FMD provides sodium (e.g., sodium chloride) in an amount that less than or equal to, in increasing order of preference, 1000 mg per day, 900 mg per day, 800 mg per day, 700 mg per day, 600 mg per day, or 500 mg per day.

In a variation, an FMD and diet package thereof that can be used to treat kidney disease as set forth above is provided in US Pat. Publication 20180228198, filed Feb. 14, 2017; the entire disclosure of which is hereby incorporated by reference. In a variation, this FMD is formed with reduced or no sodium added to form a low sodium FMD. In some refinements, the amounts of salt and/or the ranges of salt are increasing order of preference, 50%, 40%, 30%, 20%, 10%, 5%, or 1% of the amounts listed in US Pat. Publication 20180228198.

In a refinement, the low sodium fasting-mimicking diet package provides daily meal portions for a predetermined number of days to treat kidney disease as set forth above. Typically, the predetermined number of days is from 1 to 10 days (i.e., 1, 2, 3, 4, 5, 6, 7, 8, 9, or 10 days). In a particularly useful variation, the predetermined number of days is 5 or 6 days. In some variations, the low sodium fasting-mimicking diets set forth herein provide a subject at most, in increasing order of preference, 75%, 50%, 40%, 30%, or 10% of the subject's normal caloric intake or the daily recommended caloric intake for a subject. In a refinement, the low sodium fasting-mimicking diet provides at least, in increasing order of preference, 5%, 10%, or 20% of the subject's normal caloric intake or the daily recommended caloric intake for a subject. The subject's normal caloric intake is the number of kcal that the subject consumes to maintain his/her weight. The subject's normal caloric intake may be estimated by interviewing the subject or by consideration of a subject's weight. As a rough guide, the subject's normal caloric intake is on average 2600 kcal/day for men and 1850 kcal/day for women. In certain instances, the low sodium fasting-mimicking diet provides the subject with 700 to 1200 kcal/day. In a particularly useful refinement, the low sodium fasting-mimicking diet provides a male subject of average weight with about 1100 kcal/day and a female subject of average weight with 900 kcal/day. In some variations, the diet from the diet package is administered on consecutive days. In another variation, the daily meal portions are provided for only one day a week for at least a month.

In a variation as set forth in U.S. Patent Publication 20180228198, a fasting-mimicking diet package includes a kale cracker composition, a first vegetable broth composition, a mushroom soup composition, a tomato soup composition, a quinoa-containing minestrone soup composition, a bean-containing minestrone soup composition, and a pumpkin soup composition. Characteristically, the daily meal portions are packaged into meal servings or into a total daily serving to be divided into meals. In a refinement, the fasting-mimicking diet package further includes a nut-containing nutrition bar, a cocoa-containing nutrition bar, a first olive-containing composition, a first vegetable broth composition, a tea composition that includes spearmint, an energy drink composition, a micronutritional composition, and an algal oil composition. In a further refinement, the fasting-mimicking diet package further includes a second olive-containing composition, a second vegetable broth composition, a tea composition that includes spearmint and lemon, and a tea composition that includes hibiscus. In a refinement, the amounts of salt and/or the ranges of salt in each of these components are increasing order of preference, 50%, 40%, 30%, 20%, 10%, 5%, or 1% of the amounts listed in US Pat. Publication 20180228198; the entire disclosure of which is hereby incorporated by reference.

In a variation of the embodiments set forth above, the fasting-mimicking diet package includes daily meal portions that provide less than 40 grams of sugar for day 1, less than 30 grams of sugar for days 2 to 5 and any remaining days, less than 28 grams of protein for day 1, less than 18 grams of protein for days 2 to 5 and any remaining days, 20-30 grams of monounsaturated fats or more to reach the desired caloric intake (i.e., a predetermined caloric intake) for day 1, 6-10 grams of polyunsaturated fats or more to reach the desired caloric intake for day 1, 2-12 grams of saturated fats or more to reach the desired caloric intake for day 1, 10-15 grams of monounsaturated fats or more to reach the desired caloric intake for days 2 to 5 and any remaining days, 3-5 grams of polyunsaturated fats or more to reach the desired caloric intake for days 2 to 5 and any remaining days, 1-6 grams of saturated fats or more to reach the desired caloric intake for days 2 to 5, or any remaining days, and a micronutrient composition on each day and any remaining days.

In another variation of the embodiments set forth above, the fasting-mimicking diet package includes daily meal portions 8-10 kcal per kilogram body weight for each diet day. In this variation, the fasting-mimicking diet provides less than 30 grams of sugar for each diet day, less than 18 grams of protein for each diet day, 9-15 grams of monounsaturated fats or more to reach the desired caloric intake for each diet day, and 2.5-4.5 grams of polyunsaturated fats or more to reach the desired caloric intake for each diet day and 1-5.5 grams of saturated fats or more to reach the desired caloric intake for each diet day. Higher levels of the fats listed above can be provided for higher FMD formulation providing up to 100% of the normal caloric intake to subjects.

In still another variation of the embodiments set forth above, the fasting-mimicking diet package includes daily meal portions that provide 5-8 kcal per kilogram body weight for each diet day. In this variation, the fasting-mimicking diet provides less than 20 grams of sugar for each diet day, less than 12 grams of protein for each diet day, and 6.5-10 grams of monounsaturated fats or more to reach the desired caloric intake for each diet day, 2.5-4.5 grams of polyunsaturated fats or more to reach the desired caloric intake for each diet day and 1.5-4 grams of saturated fats or more to reach the desired caloric intake for each diet day.

In still another variation of the embodiments set forth above, the fasting-mimicking diet package includes daily meal servings that provide 0-3 kcal per kilogram body weight for each diet day. In this variation, the fasting-mimicking diet provides less than 5 grams of sugar for each diet day, less than 3 grams of protein for each diet day, and less than 2.5 grams of monounsaturated fats for each diet day, less than 1 grams of polyunsaturated fats for each diet day and less than 1 grams of saturated fats for each diet day.

In an embodiment, the nutritional requirements for the fasting-mimicking diet set forth above can be realized by a diet package with certain specific meal components. In one variation, the fasting-mimicking diet package 10 provides daily meal portions for a predetermined number of days that are set forth above (e.g., 1, 2, 3, 4, 5, 6, 7, 8, 9, or 10 days). The fasting-mimicking diet package 10 includes a kale cracker composition, a first vegetable broth composition, a mushroom soup composition, a tomato soup composition, a quinoa-containing minestrone soup composition, a bean-containing minestrone soup composition, and a pumpkin soup composition. Characteristically, the daily meal portions are packaged into meal servings or into a total daily serving to be divided into meals. In a refinement, the fasting-mimicking diet package further includes a nut-containing nutrition bar, a cocoa-containing nutrition bar, a first olive-containing composition, a first vegetable broth composition (item 32), a tea composition that includes spearmint, an energy drink composition, a micronutritional composition, and an algal oil composition. In a further refinement, the fasting-mimicking diet package further includes a second olive-containing composition, a second vegetable broth composition, a tea composition that includes spearmint and lemon, and a tea composition that includes hibiscus. It should be appreciated that each of the soup, broth, tea, and energy compositions set forth herein are designed to have added water when consumed.

In another variation of a fasting-mimicking diet package, diet package 10 includes a nut-containing nutrition bar, a cocoa-containing nutrition bar, a first olive-containing composition (item 30), a kale cracker composition, a vegetable soup composition, a first vegetable broth composition, a tea composition that includes spearmint, an energy drink composition (item 36), a micronutritional composition, and an algal oil composition. Characteristically, the daily meal portions are packaged into meal servings or into a total daily serving to be divided into meals. This diet package also includes daily meal portions for a predetermined number of days as set forth above, with the daily meal portions being packaged into meal servings or into a total daily serving to be divided into meals. In a refinement, the fasting-mimicking diet package further includes a mushroom soup composition, a tomato soup composition, a quinoa-containing minestrone soup composition, and a pumpkin soup composition. In a further refinement, the fasting-mimicking diet package further includes a second olive-containing composition, a second vegetable broth composition, a bean-containing minestrone soup composition, a tea composition that includes spearmint and lemon, and a tea composition that includes hibiscus.

As set forth above, the fasting-mimicking diet packages include specific meal components. Typically, compositions are as follows. The nut-containing nutrition bar includes almond meal and macadamia nuts. The cocoa-containing nutrition bar includes almond butter, almonds, and brown rice crispy (e.g., brown puffed rice). The mushroom soup composition includes brown rice powder, carrots, inulin, and mushrooms. The bean-containing minestrone soup composition includes white beans, cabbage, and potatoes. The first vegetable broth composition includes carrots, maltodextrin, celery, spinach, and tomatoes. The second vegetable broth composition includes carrots, maltodextrin, celery, spinach, soy lecithin, and tomatoes. The energy drink composition includes glycerin and water. The algal oil composition includes schizochytrium algae oil. The micronutrient composition includes beet root powder, calcium carbonate, carrots, collard leaf, kale leaf, and tomatoes. In a refinement, the micronutrient composition includes Vit A, Vit C, Ca, Fe, Vit D3, Vit E, Vit K, Vit B1, Vit B2, Vit B3, Vit B5, Vit B6, Vit B7, Vit B9, Vit B12, Cr, Cu, I, Mg, Mn, Mo, Se, and Zn.

In a refinement, the nut-containing nutrition bar (L-Bar Nut based) includes almond meal and macadamia nuts. In a refinement, the nut-containing nutrition bar (L-Bar Nut based) includes almond meal preferably in an amount of 20 to 35 weight %; coconut preferably in an amount of 2 to 10 weight %; coconut oil preferably in an amount of 1 to 8 weight %; flax seed meal preferably in an amount of 1 to 8 weight %; honey preferably in an amount of 10 to 30 weight %; macadamia nuts preferably in an amount of 10 to 30 weight %; pecans preferably in an amount of 10 to 25 weight %; salt preferably in an amount of 0.0 to 0.8 weight % (e.g., 0.0.2 to 0.2 weight % or 0.1 to 0.8 weight %); and optionally vanilla preferably in an amount of 0.3 to 1.5 weight %.

In a refinement, the cocoa-containing nutrition bar (L-Bar ChocoCrisp) includes almond butter, almonds, and brown rice crispy (PGP10235). In a refinement, the cocoa-containing nutrition bar (L-Bar ChocoCrisp) includes almond butter preferably in an amount of 10 to 25 weight %; almonds preferably in an amount of 3 to 12 weight %; brown rice crispy (PGP10235) preferably in an amount of 10 to 25 weight %; brown rice syrup preferably in an amount of 2 to 8 weight %; chocolate liquor preferably in an amount of 1 to 4 weight %, cocoa butter preferably in an amount of 0.4 to 1.6 weight %; cocoa powder preferably in an amount of 4 to 12 weight %; fiber syrup SF75 preferably in an amount of 18 to 38 weight %, flaxseed oil preferably in an amount of 1 to 3 weight %; salt preferably in an amount of 0.0 to 0.4 weight % (0.1 to 0.4 weight % or 0.05 to 0.1 weight percent) and sugar preferably in an amount of 1 to 6 weight %.

In a refinement, the first olive-containing composition (sea salt version) includes olives, olive oil, and sea salt. In a refinement, the first olive-containing composition (sea salt) includes lactic acid preferably in an amount of 0.3 to 1 weight %; oil (olive) preferably in an amount of 2 to 6 weight %; olives (raw, green pitted) preferably in an amount of 50 to 97 weight %; salt (reg., kosher, sea salt) preferably in an amount of 0.0 to 3 weight % (e.g., 0.2 to 0.8 weight percent or 0.8 to 3 weight %); and thyme preferably in an amount of 0.1 to 0.5 weight %.

In a refinement, the second olive-containing composition (garlic version) includes olives, olive oil, and garlic. In a refinement, the second olive-containing composition (garlic) includes garlic preferably in an amount of 0.1 to 0.6 weight %; lactic acid preferably in an amount of 0.3 to 1 weight %; oil (olive) preferably in an amount of 2 to 6 weight %; olives (raw, green pitted) preferably in an amount of 50 to 97 weight %; salt (reg., kosher, sea salt) preferably in an amount of 0.0 to 3 weight % (e.g., 0.2 to 1 weight percent or 0.8 to 3 weight %); thyme preferably in an amount of 0.1 to 0.5 weight %.

In a refinement, the kale cracker composition includes kale, almonds, tapioca flour, and optionally sesame seeds. In another refinement, the kale cracker composition includes almonds preferably in an amount of 15 to 40 weight %; black pepper preferably in an amount of 0.1 to 0.4 weight %; chia seeds preferably in an amount of 3 to 10 weight %; chili pepper preferably in an amount of 0.4 to 1.2 weight %; cumin seeds preferably in an amount of 0.3 to 0.9 weight %; flax seeds preferably in an amount of 3 to 10 weight %; garlic preferably in an amount of 0.02 to 0.04 weight %; kale preferably in an amount of 2 to 6 weight %; oil (sunflower) preferably in an about of 2 to 7 weight %; onion (powder, minced) typically in an amount of 0.3 to 0.9 weight %; oregano preferably in an amount of 0.01 to 0.06 weight %; salt preferably in an amount of 0 to 4 weight % (e.g., 0.2 to 1 weight percent or 1 to 4 weight %); sesame seeds preferably in an amount of 15 to 35 weight %; sugar (coconut) preferably in an amount of 1 to 5 weight %; tapioca flour preferably in an amount of 10 to 30 weight %; vinegar (coconut) preferably in an amount of 1 to 4 weight %; water (purified) preferably in an amount of 2 to 12 weight %; and yeast extract preferably in an amount of 0.3 to 1 weight %.

In another refinement, the kale cracker composition includes kale, golden flax seeds, sesame seeds, and sunflower seeds. In another refinement, the kale cracker composition includes apple cider vinegar preferably in an amount 1 to 3 weight %; black pepper preferably in an amount of 0.4 to 1.3 weight %; cashews preferably in an amount of 4 to 13 weight %; dill weed preferably in an amount of 0.4 to 1.3 weight %; flax seeds golden preferably in an amount of 13 to 40 weight %; hemp seeds preferably in an amount of 0.7 to 2 weight %; kale preferably in an amount of 14 to 42 weight %; onion, white, dried, (powder, minced) preferably in an amount of 0.5 to 1.6 weight %; pumpkin seeds preferably in an amount of 0.7 to 2 weight %; salt (reg; kosher, sea salt) preferably in an amount of 0.0 to 2 weight %; (e.g., 0.2 to 0.5 weight percent or 0.7 to 2 weight %); Sesame seeds preferably in an amount of 2 to 8 weight %; sunflower seeds preferably in an amount of 10 to 30 weight %; and yeast extract preferably in an amount of 1 to 5 weight %.

In a refinement, the vegetable soup composition includes onions, tomatoes, spinach, green tea extract, optionally rice flour, optionally brown rice powder, optionally carrots, and optionally inulin, leeks, In a refinement, the vegetable soup composition includes basil (whole leaf, dried) preferably in an amount of 0.3 to 0.9 weight %; brown rice powder (whole grain) preferably in an amount of 3 to 12 weight %; carrot (dehydrated, puffed, powder, pieces) preferably in an amount of 4 to 14 weight %; green tea extract preferably in an amount of 0.02 to 0.06 weight %; inulin preferably in an amount of 5 to 15 weight %; leeks (granules −10+40) preferably in an amount of 1 to 5 weight %; oil (olive) preferably in an amount of 1 to 6 weight %; onion (powder, minced) preferably in an amount of 4 to 15 weight %; parsley preferably in an amount of 0.3 to 0.8 weight %; red bell peppers preferably in an amount of 1 to 5 weight %; rice flour preferably in an amount of 18 to 50 weight %; salt preferably in an amount of 1 to 7 weight % (e.g., 2 to 7 weight % or 0.5 to 2 weight percent; spinach (leaf, powder) preferably in an amount of 0.4 to 1.5 weight %; tomatoes (fruit powder, sun dried, granules) preferably in an amount of 4 to 14 weight %; yeast extract preferably in an amount of 0.5 to 1.8 weight %. In the vegetable soup composition and any of the compositions set forth herein having rice flour, the rice flour can be glutinous or non-glutinous, milled, or unmilled.

In another refinement, the vegetable soup composition includes carrots, inulin, leeks, onions, and rice flour. In a refinement, the vegetable soup composition includes basil, whole leaf, dried preferably in an amount of 0.3 to 1 weight %; carrot (dehydrated, puffed, powder, pieces) preferably in an amount of 4 to 12 weight %; inulin preferably in an amount of 6 to 18 weight %; leeks in an amount of 1 to 5 weight %; oil (olive) preferably in an amount of 1 to 3 weight %; Onion, white, dried, (powder, minced) preferably in an amount of 10 to 30 weight %; parsley preferably in an amount of 0.3 to 1 weight %; potato preferably in an amount of 1 to 5 weight %; red pepper preferably in an amount of 1 to 6 weight %; rice flour in an amount of 13 to 40 weight %; salt (reg., kosher, sea salt) in an amount of 0 to 12 weight % (e.g., 1 to 3 weight percent or 4 to 12 weight %); spinach (leaf, powder) preferably in an amount of 0.2 to 1 weight %; and tomatoes, (fruit powder, sun dried granules) preferably in an amount of 3 to 13 weight %.

In a refinement, the mushroom soup composition includes mushrooms, green tea extract, optionally brown rice powder, optionally carrots, and optionally inulin. In a refinement, the mushroom soup composition includes brown rice powder (whole grain) preferably in an amount of 10 to 30 weight %; carrot (dehydrated, puffed, powder, pieces) preferably in an amount of 3 to 12 weight %; green tea extract preferably in an amount of 0.02 to 0.06 weight %; inulin preferably in an amount of 3 to 12 weight %; mushrooms (European mix, powder, pieces) preferably in an amount of 6 to 18 weight %; oil (olive) preferably in an amount of 1 to 6 weight %; onion preferably in an amount of powder, minced) preferably in an amount of 3 to 12 weight %; parsley preferably in an amount of 0.1 to 0.5 weight %; rice flour preferably in an amount of 18 to 50 weight %; salt preferably in an amount of 2 to 8 weight % (e.g., 2 to 8 weight % or 0.5 to 2 weight percent); yeast extract preferably in an amount of 0.5 to 1.5 weight %.

In another refinement, the mushroom soup composition includes carrots, inulin, mushrooms, onions, and rice flour. In another refinement, the mushroom soup composition includes carrot (dehydrated, puffed, powder, pieces) preferably in an amount of 7 to 22 weight %; inulin preferably in an amount of 7 to 22 weight %; mushrooms (European mix), (powder & pieces)dehydrated preferably in an amount of 7 to 22 weight %; oil (olive) preferably in an amount of 0.6 to 2 weight %; Onion, white, dried, (powder, minced) preferably in an amount of 7 to 22 weight %; parsley preferably in an amount of 0.3 to 0.9 weight %; potato preferably in an amount of 0.6 to 2 weight %; rice flour preferably in an amount of 15 to 45 weight %; salt (reg., kosher, sea salt) preferably in an amount of 0 to 18 weight % (e.g., 1 to 4 weight percent or 6 to 18 weight %); and yeast extract preferably in an amount of 0.7 to 2.2 weight %.

In a refinement, the tomato soup composition includes tomatoes, green tea extract, optionally inulin, and optionally onions. In a refinement, the tomato soup composition (new) includes basil (whole leaf, dried) preferably in an amount of 0.2 to 0.7 weight %; brown rice powder (whole grain) preferably in an amount of 1 to 5 weight %; green tea extract preferably in an amount of 0.02 to 0.06 weight %; inulin preferably in an amount of 7 to 20 weight %; oil (olive) preferably in an amount of 3 to 9 weight %; onion preferably (powder, minced) preferably in an amount of 4 to 12 weight %; parsley preferably in an amount of 0.1 to 0.6 weight %; rice flour preferably in an amount of 18 to 50 weight %; salt preferably in an amount of 2 to 9 weight % (e.g., 0.5 to 2 weight % or 2 to 9 weight %); tomatoes (fruit powder, sun dried, granules) preferably in an amount of 12 to 36 weight %; and yeast extract preferably in an amount of 0.5 to 3 weight 0.

In another refinement, the tomato soup composition includes tomatoes, inulin, olives, onions, potatoes, and rice flour. In still another refinement, the tomato soup composition includes basil, whole leaf, dried preferably in an amount of 0.3 to 1 weight %; inulin preferably in an amount of 6 to 18 weight %; oil (olive) preferably in an amount of 4 to 14 weight %; onion, white, dried, (powder, minced) preferably in an amount of 8 to 24 weight %; parsley preferably in an amount of 0.3 to 0.9 weight %; potato preferably in an amount of 6 to 18 weight %; rice flour preferably in an amount of 9 to 27 weight %; salt (reg., kosher, sea salt) preferably in an amount of 4 to 14 weight % (e.g., 1 to 3 weight % or 4 to 14 weight %); tomatoes, (fruit powder, sun dried granules) preferably in an amount of 8 to 24 weight %; and yeast extract preferably in an amount of 0.7 to 2.2 weight %.

In a refinement, the quinoa-containing minestrone soup composition includes quinoa, green tea extract, optionally olive oil, optionally cabbage, optionally potatoes, optionally rice flour, and optionally tomatoes and optionally no turmeric. In a refinement, the quinoa-containing minestrone soup composition includes basil (whole leaf, dried preferably in an amount of 0.7 to 2 weight %; broccoli powder preferably in an amount of 0.6 to 2 weight %; cabbage white (flakes) preferably in an amount of 3 to 10 weight %; carrot (dehydrated, puffed, powder, pieces) preferably in an amount of 3 to 10 weight %; celery preferably in an amount of 1 to 4 weight %; celery seeds (powder) preferably in an amount of 0.07 to 0.2 weight %; garlic preferably in an amount of 0.7 to 2 weight %; green tea extract preferably in an amount of 0.02 to 0.06 weight %; inulin preferably in an amount of 1 to 5 weight %; leeks (granules −10+40), preferably in an amount of 0.7 to 2 weight %; oil (olive) preferably in an amount of 0.6 to 2 weight %; onion (powder, minced) preferably in an amount of 2 to 8 weight %; peas preferably in an amount of 3 to 10 weight %; potato preferably in an amount of 7 to 20 weight %; quinoa preferably in an amount of 7 to 20 weight %; rice flour preferably in an amount of 7 to 20 weight %; salt, preferably in an amount of 1 to 6 weight % (e.g., 0.2 to 1.5 weight % or 1 to 6 weight %); spinach (leaf, powder) preferably in an amount of 0.5 to 2 weight %; tomatoes (fruit powder, sun dried, granules) preferably in an amount of 2 to 6 weight %; yeast extract preferably in an amount of 0.6 to 2 weight %; zucchini (powder, diced) preferably in an amount of 2 to 8 weight %.

In another refinement, the quinoa-containing minestrone soup includes quinoa, cabbage, potatoes, and rice flour. In still another refinement, the quinoa-containing minestrone soup includes basil, whole leaf, dried preferably in an amount of 0.7 to 2.2 weight %; broccoli powder preferably in an amount of 0.7 to 2.2 weight %; cabbage white (flakes) preferably in an amount of 0.6 to 2.2 weight %; carrot (dehydrated, puffed, powder, pieces) preferably in an amount of 3 to 10 weight %; celeriac preferably in an amount of 2 to 6 weight %; celery seeds powder preferably in an amount of 0.6 to 1.8 weight %; garlic preferably in an amount of 1 to 3 weight %; Onion, white, dried, (powder, minced) preferably in an amount of 3 to 9 weight %; peas preferably in an amount of 3 to 10 weight %; potato preferably in an amount of 6 to 20 weight %; quinoa preferably in an amount of 8 to 23 weight %; rice flour preferably in an amount of 7 to 22 weight %; salt (reg., kosher, sea salt) preferably in an amount of 2 to 7 weight % (e.g., 0.5 to 2 weight % or 2 to 7 weight %); savoy cabbage preferably in an amount of 3 to 10 weight %; spinach (leaf, powder) preferably in an amount of 0.7 to 2.2 weight %; turmeric preferably in an amount of 0.6 to 1.8 weight %; yeast extract preferably in an amount of 3 to 10 weight %; and zucchini (powder, diced) preferably in an amount of 1 to 5 weight %.

In a refinement, the bean-containing minestrone soup composition includes white beans (e.g., great northern beans), great tea extract, optionally cabbage, and optionally potatoes. In a refinement, the bean-containing minestrone soup composition includes beans (great northern) preferably in an amount of 3 to 10 weight %; cabbage white (flakes) preferably in an amount of 2 to 8 weight %; carrot (dehydrated, puffed, powder, pieces) preferably in an amount of 2 to 8 weight %; celery preferably in an amount of 1 to 4 weight %; green tea extract preferably in an amount of 0.02 to 0.06 weight %; inulin preferably in an amount of 2 to 10 weight %; leeks (granules −10+40) preferably in an amount of 2 to 7 weight %; oil (olive) preferably in an amount of 2 to 7 weight %; onion (powder, minced) preferably in an amount of 2 to 7 weight %; parsley preferably in an amount of 0.2 to 1 weight %; peas preferably in an amount of 3 to 9 weight %; potato preferably in an amount of 15 to 45 weight %; rice flour preferably in an amount of 6 to 18 weight %; salt preferably in an amount of 2 to 8 weight % (e.g., 0.5 to 2 weight % or 2 to 8 weight %); spinach (leaf, powder) preferably in an amount of 0.5 to 1.5 weight %; tomatoes (fruit powder, sun dried, granules) preferably in an amount of 2 to 7 weight %; and yeast extract preferably in an amount of 0.5 to 1.5 weight %.

In a refinement, the bean-containing minestrone soup composition includes brown beans, carrots, peas, potato, and rice flour. In another refinement, the bean-containing minestrone soup composition includes carrot (dehydrated, puffed, powder, pieces) preferably in an amount of 4 to 14 weight %; celeriac preferably in an amount of 1 to 5 weight %; celery preferably in an amount of 0.5 to 1.6 weight %; leeks preferably in an amount of 2 to 8 weight %; oil (olive) preferably in an amount of 2 to 8 weight %; Onion, white, dried, (powder, minced) preferably in an amount of 3 to 10 weight %; parsley preferably in an amount of 0.5 to 1.5 weight %; peas preferably in an amount of 5 to 18 weight %; potato preferably in an amount of 8 to 24 weight %; rice flour preferably in an amount of 5 to 18 weight %; salt (reg., kosher, sea salt) preferably in an amount of 4 to 14 weight % (e.g., 1 to 3 weight % or 4 to 14 weight %); spinach (leaf, powder) preferably in an amount of 0.5 to 1.5 weight %; tomatoes, (fruit powder, sun dried granules) preferably in an amount of 0.9 to 2.8 weight %; turmeric preferably in an amount of 0.3 to 1.2 weight %; and yeast extract preferably in an amount of 0.5 to 1.5 weight %.

In a refinement, the pumpkin soup composition includes pumpkin, green tea extract, optionally rice flour, optionally carrots, and optionally brown rice powder. In a refinement, the pumpkin soup composition includes (new) includes brown rice powder (whole grain) preferably in an amount of 3 to 9 weight %; carrot (dehydrated, puffed, powder, pieces) preferably in an amount of 2 to 8 weight %; green tea extract preferably in an amount of 0.02 to 0.06 weight %; inulin preferably in an amount of 2 to 10 weight %; oil (olive) preferably in an amount of 1 to 7 weight %; onion (powder, minced) preferably in an amount of 1.0 to 3 weight %; pumpkin powder preferably in an amount of 20 to 60 weight %; rice flour preferably in an amount of 15 to 45 weight %; salt preferably in an amount of 2 to 10 weight % (e.g., 0.5 to 2.5 weight % or 2 to 10 weight %); and yeast extract preferably in an amount of 0.3 to 1 weight %.

In a refinement, the first vegetable broth includes carrots, maltodextrin, celery, spinach, and tomatoes. In a refinement, the first vegetable broth includes carrot (dehydrated, puffed, powder, pieces) preferably in an amount of 6 to 18 weight %; celery preferably in an amount of 3 to 10 weight %; garlic preferably in an amount of 3 to 10 weight %; maltodextrin preferably in an amount of 8 to 25 weight %; oil (canola) preferably in an amount of 0.5 to 2 weight %; onion (powder, minced) preferably in an amount of 6 to 18 weight %; parsley preferably in an amount of 3 to 10 weight %; potato preferably in an amount of 1 to 3 weight %; salt preferably in an amount of 0 to 21 weight % (e.g., 1.5 to 5 weight % or 7 to 21 weight %); spinach (leaf, powder) preferably in an amount of 3 to 10 weight %; tomatoes (fruit powder, sun dried, granules) preferably in an amount of 6 to 18 weight %; and yeast extract preferably in an amount of 1 to 6 weight %.

In a refinement, the second vegetable broth (chicken flavoring) includes carrots, chicken flavoring, maltodextrin, celery, spinach, soy lecithin, and tomatoes. In a refinement, the second vegetable broth composition includes carrot (dehydrated, puffed, powder, pieces) preferably in an amount of 3 to 10 weight %; celery preferably in an amount of 3 to 12 weight %; garlic preferably in an amount of 3 to 9 weight %; maltodextrin preferably in an amount of 8 to 25 weight %; oil (canola) preferably in an amount of 0.5 to 2 weight %; onion preferably in an amount of powder, minced) preferably in an amount of 3 to 12 weight %; parsley preferably in an amount of 3 to 10 weight %; potato preferably in an amount of 1 to 6 weight %; salt preferably in an amount of 8 to 25 weight % (e.g., 2 to 6 weight % or 8 to 25 weight %); soy lecithin preferably in an amount of 0.5 to 3 weight %; spinach (leaf, powder) preferably in an amount of 3 to 12 weight %; tomatoes (fruit powder, sun dried, granules) preferably in an amount of 6 to 18 weight %; xanthan gum preferably in an amount of 0.5 to 4 weight %; and yeast extract preferably in an amount of 4 to 12 weight %.

In a refinement, the energy drink composition comprises glycerin and purified water as principal components. The glycerin is preferably present in an amount of about 20 to 60 weight percent, more preferably about 30 to 50 weight percent, and serves as both a humectant and an energy substrate. The purified water is preferably present in an amount of about 40 to 80 weight percent, more preferably about 50 to 70 weight percent, providing the aqueous carrier phase for the composition and ensuring proper solubility and stability of other ingredients.

In a refinement, the tea composition comprises organic spearmint leaves as the primary botanical component. The spearmint leaves are preferably present in an amount of about 70 to 100 weight percent, more preferably about 80 to 95 weight percent, based on the total weight of the tea composition. The organic spearmint provides the characteristic aroma and flavor profile and contributes antioxidant and digestive-supportive properties to the beverage.

In a refinement, the tea composition comprising lemon and spearmint includes organic lemon myrtle, organic lemon peel, and organic spearmint leaves. The lemon myrtle is preferably present in an amount of about 3 to 12 weight percent, more preferably about 5 to 10 weight percent. The lemon peel is preferably present in an amount of about 10 to 25 weight percent, more preferably about 15 to 20 weight percent. The spearmint leaves are preferably present in an amount of about 50 to 95 weight percent, more preferably about 70 to 90 weight percent, based on the total weight of the tea composition. The combination provides a balanced aromatic profile with complementary citrus and mint characteristics that enhance flavor and bioactive potential.

In a refinement, the tea composition comprising hibiscus includes organic hibiscus tea leaves as the primary botanical component. The hibiscus tea leaves are preferably present in an amount of about 80 to 100 weight percent, more preferably about 85 to 95 weight percent, based on the total weight of the tea composition. The organic hibiscus provides a naturally tart flavor profile and contributes antioxidant and circulatory-supportive properties to the beverage.

In a refinement, the algal oil composition comprises Schizochytrium algal oil rich in docosahexaenoic acid (DHA) omega-3 fatty acids. The Schizochytrium algal oil is preferably present in an amount of about 80 to 100 weight percent, more preferably about 85 to 95 weight percent, based on the total weight of the algal oil composition. The algal oil serves as a plant-derived source of long-chain omega-3 fatty acids that support cognitive, cardiovascular, and cellular health.

In a refinement, the nutrient replenishment composition (NR-1) includes beetroot powder, calcium carbonate, carrots, collard leaf, kale leaf, and tomatoes. In a refinement, the nutrient replenishment composition (NR-1) includes ascorbic acid preferably in an amount of 1 to 3 weight %; beet root powder preferably in an amount of 6 to 20 weight %; beta carotene preferably in an amount of 0.05 to 0.15 weight %; calcium carbonate preferably in an amount of 6 to 20 weight %; carrot (dehydrated, puffed, powder, pieces) preferably in an amount of 6 to 20 weight %; cholecaliciferol preferably in an amount of 0.00 weight %; chromuim Picolinate preferably in an amount of 0.00 weight %; collard leaf powder preferably in an amount of 6 to 20 weight %; cupric sulfate preferably in an amount of 0.01 to 0.06 weight %; cyanocobalamin, 0.00; Dl-alpha tocopherol acetate preferably in an amount of 0.3 to 1 weight %; ferrous fumarate preferably in an amount of 0.2 to 1 weight %; folic acid preferably in an amount of 0.00 weight %; kale leaf preferably in an amount of 6 to 20 weight %; magnesium stearate preferably in an amount of 1 to 6 weight %; manganese sulfate preferably in an amount of 0.04 to 0.08 weight %; niacinamide preferably in an amount of 0.3 to 1 weight %; pantothenic acid preferably in an amount of 0.1 to 0.6 weight %; phytonadione preferably in an amount of 0.00 weight %; potassium iodine preferably in an amount of 0 weight %; pyriodoxine HCl preferably in an amount of 0.03 to 0.1 weight %; riboflavin preferably in an amount of 0.02 to 0.1 weight %; sodium molybdate preferably in an amount of 0.00 weight %; sodium selenate preferably in an amount of 0.00 weight %; spinach (leaf, powder) preferably in an amount of 6 to 20 weight %; thiamine mononitrate preferably in an amount of 0.02 to 0.1 weight %; tomatoes (fruit powder, sun dried, granules) preferably in an amount of 6 to 20 weight %; tribasic calcium phosphate preferably in an amount of 0.5 to 2 weight %; and zinc oxide preferably in an amount of 0.2 to 0.8 weight %.

In a variation, each of the components of the fasting-mimicking diet package and therefore the fasting-mimicking diet, is substantially gluten free (e.g., each component has less than 20 ppm gluten) or exceptionally low gluten (e.g., each component has 20-100 ppm). In other variations, each of the components are provided in a serving size from 20 to 60 g. In other variations, the nut-containing nutrition bar is provided in a serving size from 30 to 60 g; cocoa-containing nutrition bar is provided in a serving size from 15 to 40 g; the olive containing composition (sea salt version) in a serving size from 10 to 20 g; the olive containing composition (garlic version) in a serving size from 10 to 20 g; kale cracker composition is provides in a serving size from 30 to 60 g; In another variation, the kale cracker compositions are provided in a serving size from 20 to 50 g; the vegetable soup compositions are provided in a serving size from 20 to 50 g; the mushroom soup compositions are provided in a serving size from 20 to 50 g; the tomato soup compositions are provided in a serving size from 20 to 50 g; the bean-containing minestrone soup compositions are provided in a serving size from 20 to 50 g; the quinoa-containing minestrone soup compositions are provided in a serving size from 20 to 50 g; the pumpkin soup compositions are provided in a serving size from 20 to 50; the first vegetable broth compositions are provided in a serving size from 5 to 15; the second vegetable broth compositions are provided in a serving size from 3 to 15; and Energy Drink composition is provided in serving size of 1 to 5 oz.

As set forth above, it was pointed out that although low sodium ketogenic FMD diets are optimal for treating kidney disease, ketogenic FMD's that are not low sodium can also provide a benefit for treating kidney disease. Therefore, a standard ketogenic FMD (i.e., not low sodium) can be substituted in the descriptions of the low sodium FMD and vice versa as needed. In this context, “not low sodium” means that that the diet is not sodium restrict. For example, the FMD can include greater than 2000 mg of sodium per day and/or greater than 1500 mg of potassium per day, and greater than 1500 mg of phosphorus per day. For example, a normal FMD comprises a reduced-calorie regimen provided over a plurality of days, typically five consecutive days, followed by a refeeding period of at least seven days. The daily caloric intake may range from about 600 kcal to about 1300 kcal, with a macronutrient composition including approximately 5-15 percent of total calories from protein, 35-65 percent from fat, and 25-60 percent from carbohydrates. Sodium levels are maintained within a normal range, for example from greater than about 2 grams to about 3.5 grams per day. The FMD can be supplied as a collection of prepared food items such as soups, nut bars, vegetable broths, energy drinks, and micronutrient blends formulated to provide the desired macronutrient and caloric profile.

In a refinement, a method of treating a kidney disease in a human subject in need thereof includes a step of administering to the subject a plurality of cycles of a fasting-mimicking diet (FMD) that is not a low-sodium fasting-mimicking diet (LS-FMD). Each cycle comprising at least five consecutive days of reduced caloric intake followed by a refeeding period of at least seven days, wherein the reduced caloric intake (i.e., the normal FMD) provides on day 900 to 1300 kilocalories with 5-15% of calories from protein, 40-65% of calories from fat, and 25-50% of calories from carbohydrate; and on each of days 2-5 or any additional days, 600 to 800 kilocalories with 5-15% of calories from protein, 35-55% of calories from fat, and 40-55% of calories from carbohydrates.

In another aspect, administration of the normal FMD is effective to improve or maintain kidney function and to reduce markers of renal stress or injury. Without being bound by theory, the therapeutic effect is believed to result from a transient metabolic shift that reduces insulin-like growth factor signaling, enhances autophagy, modulates inflammatory pathways, and promotes endogenous repair mechanisms within the kidney. Such responses collectively support glomerular integrity, maintain filtration capacity, and stabilize or improve estimated glomerular filtration rate (eGFR).

In some variations, the subject receives a series of FMD cycles over a treatment period of weeks or months. Each cycle includes at least five consecutive days of the FMD followed by a refeeding period, for example 5 to 26 days. A plurality of cycles (such as three cycles administered over approximately three months) may be employed. The regimen can be repeated periodically to maintain therapeutic benefit or to prevent progression of kidney dysfunction.

In another aspect, the methods with respect to the standard FMD described herein are applicable to a variety of renal pathologies selected from, but not limited to, chronic kidney disease, diabetic nephropathy, IgA nephropathy, focal segmental glomerulosclerosis, minimal change disease, membranous nephropathy, membranoproliferative glomerulonephritis, lupus nephritis, and ischemic nephropathy. The standard FMD can be used alone or in combination with other therapeutic agents such as angiotensin-converting enzyme inhibitors, angiotensin receptor blockers, SGLT2 inhibitors, or diuretics. In a refinement, administration of the standard FMD results in one or more of the following: reduction in urinary protein or albumin excretion, stabilization or increase of eGFR relative to baseline, improvement in endothelial function, and promotion of circulating renal progenitor-like cells expressing CD24⁺CD133⁺CD45⁻ markers. These effects contribute to improved renal performance and decreased risk of further disease progression. In a further refinement, the FMD reduces proteinuria measured as 24-hour urinary protein or albumin-to-creatinine ratio and increases estimated glomerular filtration rate (eGFR) relative to baseline.

Additional details are found in Villani V, Frank C N, Cravedi P, Hou X, Bin S, Kamitakahara A, Barbati C, Buono R, Da Sacco S, Lemley K V, De Filippo R E, Lai S, Laviano A, Longo V D, Perin L. A kidney-specific fasting-mimicking diet induces podocyte reprogramming and restores renal function in glomerulopathy. Sci Transl Med. 2024 Oct. 30; 16(771):eadl5514. doi: 10.1126/scitranslmed.adl5514. Epub 2024 Oct. 30. PMID: 39475573.; the entire disclosure of which is hereby incorporated by reference.

The following examples illustrate the various embodiments of the present invention. Those skilled in the art will recognize many variations that are within the spirit of the present invention and scope of the claims.

### EXPERIMENTS

### Cycles of LS-FMD Restore Kidney Function and Preserve Normal Kidney Transcriptional Profile in PAN-Treated Rats

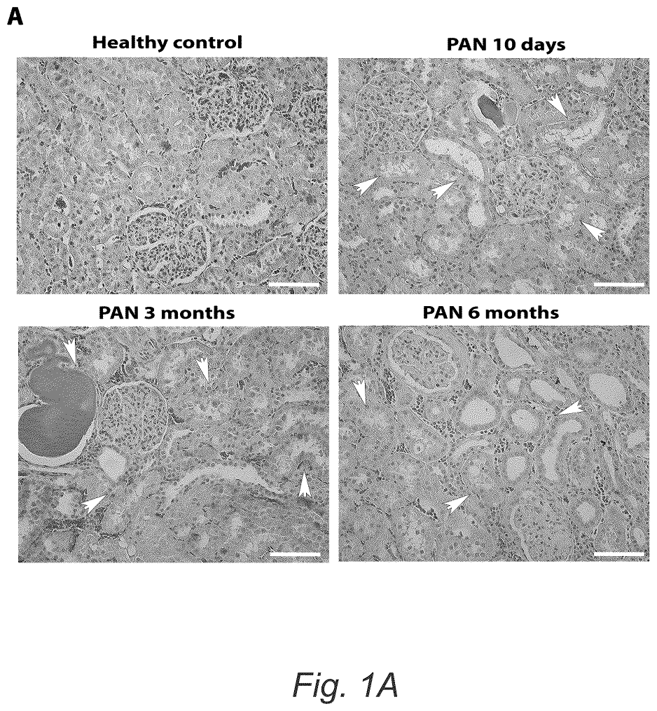

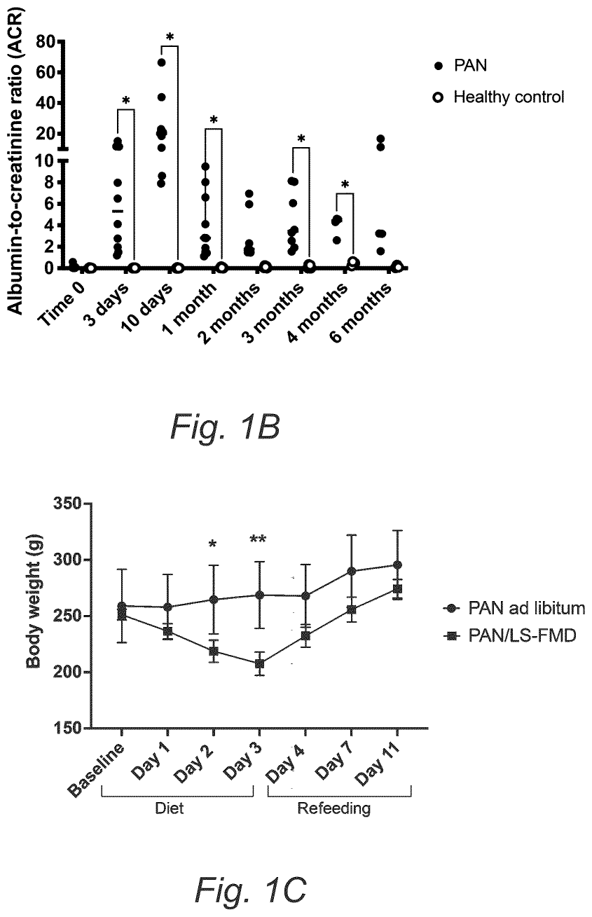

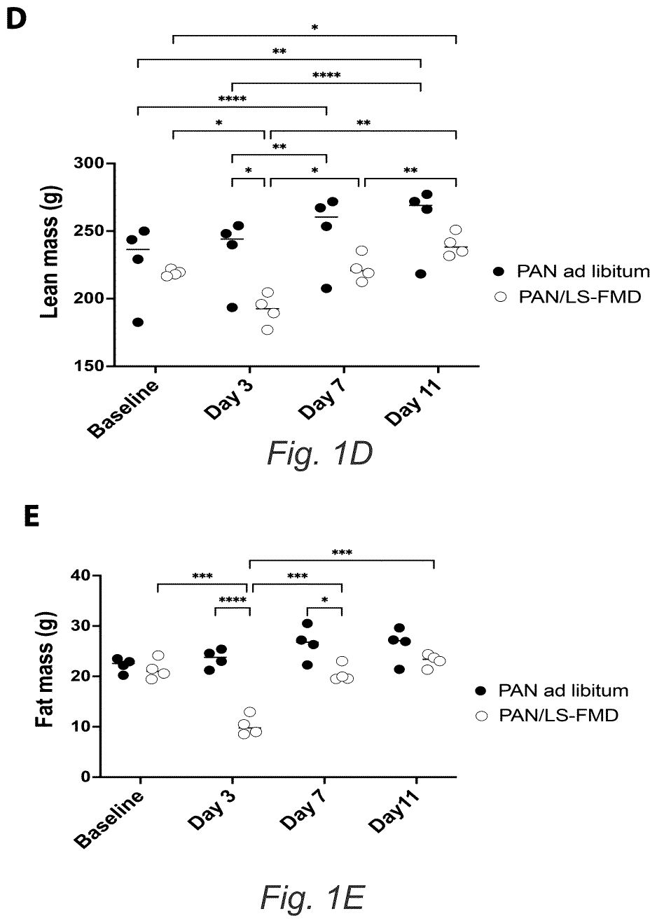

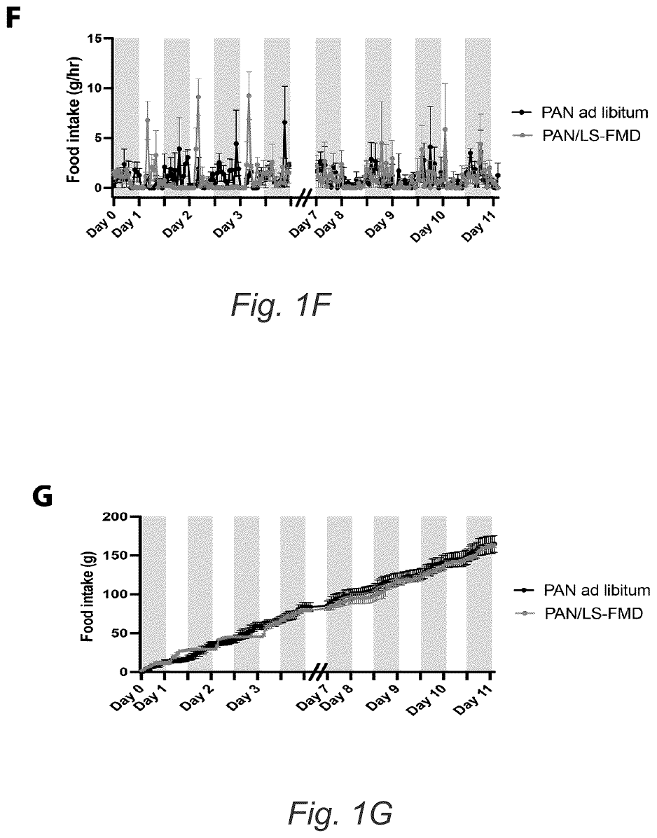

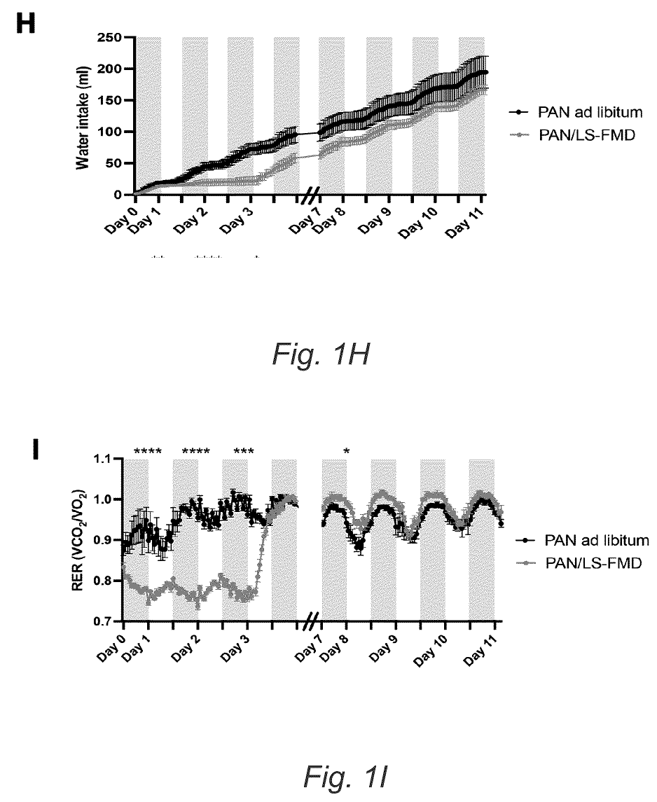

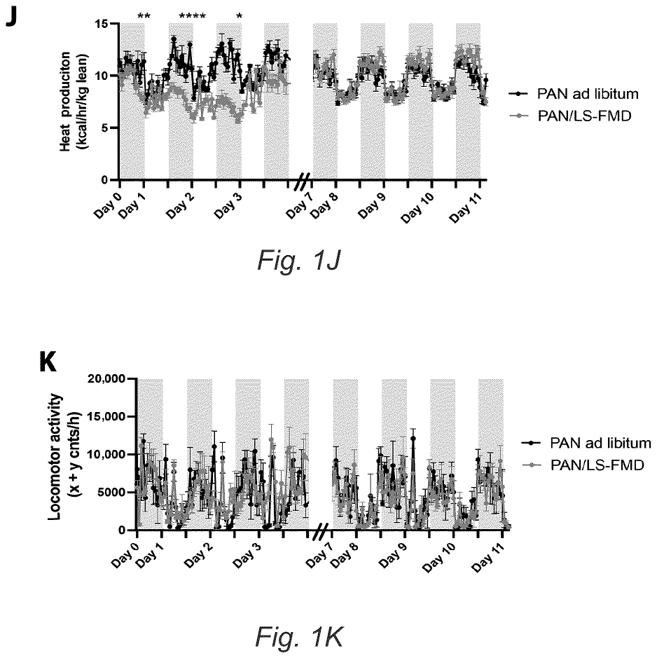

A chronic kidney disease (CKD) model of nephrosis was established in rats through administration of a single intraperitoneal injection of puromycin aminonucleoside (PAN) at a dosage of 40 mg per kilogram of body weight. Histological assessment of tubular injuries (dilation, vacuolization, loss of brush border, fibrosis, and cast formation) and glomerular [glomerulosclerotic index (GSI)] injuries revealed visible structural damage 10 days after injection, with worsening of morphological structures at 3 and 6 months (FIG. 1A). Albumin-to-creatinine ratio (ACR), as evidence of kidney injury, was also elevated, starting on day 3 and peaking at day 10. ACR values of PAN-treated rats remained elevated compared with those of healthy controls (FIG. 1B), confirming the establishment of CKD in the animal model.

To align with the recommended safe daily intake of electrolytes for patients with chronic kidney disease (CKD), a kidney-specific fasting-mimicking diet (LS-FMD) was developed and evaluated. The formulation was adjusted to modify electrolyte content (potassium (K) at 33 mg/kg, phosphorus (P) at 29.2 mg/kg, and sodium (Na) at 81.7 mg/kg (27-30)) using a human equivalent factor of 6.02 to account for interspecies differences in metabolic rate (31).

Because the FMD or LS-FMD has not been previously studied in a rat model, a metabolic study was performed to evaluate the effect of one cycle of a 4-day LS-FMD followed by regular chow in both PAN-treated and healthy rats to ensure feasibility and safety of this approach in injured rats (FIG. 1, C to K). Body weight, lean mass, and fat mass were significantly (P<0.05) reduced in rats fed with LS-FMD at the end of the diet compared with those in rats fed ad libitum but returned to the normal range after refeeding, as has been previously established in both mice and humans (FIG. 1, C to E) (20-25). Food intake per hour showed a peak in the FMD cohort starting at day 1 of the diet; animals on the LS-FMD consumed most of their food all at once; the cumulative food intake by weight remained similar between groups, whereas calorie intake was very different (FIGS. 1, F and G). Rats on the LS-FMD decreased their drinking water intake, which was compensated by the water in the hydrogel used to deliver the diet, as also shown for the regular FMD, but returned to normal water intake afterward (FIG. 1H). The respiratory exchange ratio, an index of energy substrate utilization that exhibits a circadian rhythm, indicates that rats displayed an oscillating trend with a mix of substrate energy sources with increased fat metabolism in the LS-FMD cohort that returned to normal immediately after refeeding (FIG. 1I). The LS-FMD caused a reduction in heat production or energy utilization, which also returned to normal after refeeding (FIG. 1J), but had no impact on the locomotor activity of rats (FIG. 1K). These same parameters were analyzed in healthy rats, showing a similar trend, with significant changes (P<0.05) during the LS-FMD and a return to baseline upon refeeding. Of note, PAN-treated and healthy rats responded to LS-FMD treatment similarly, and no differences were observed in weight and lean mass, as well as fat mass recovery. Together, these data were also confirmed in healthy rats treated with FMD, validate the safety of the approach, and support the anticipated effects of the LS-FMD in a rat model.

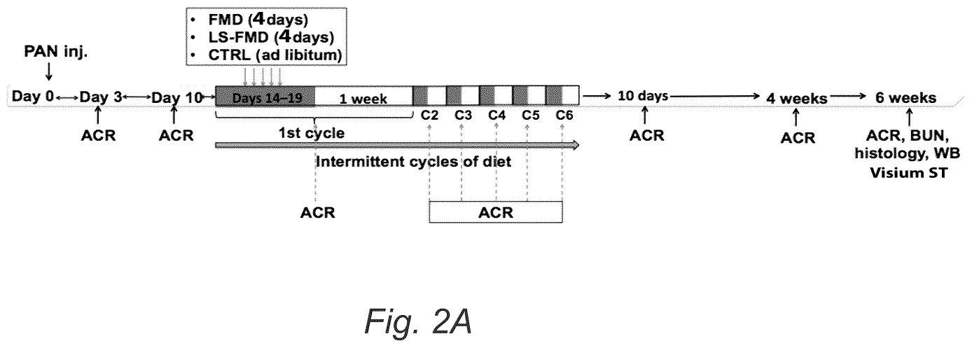

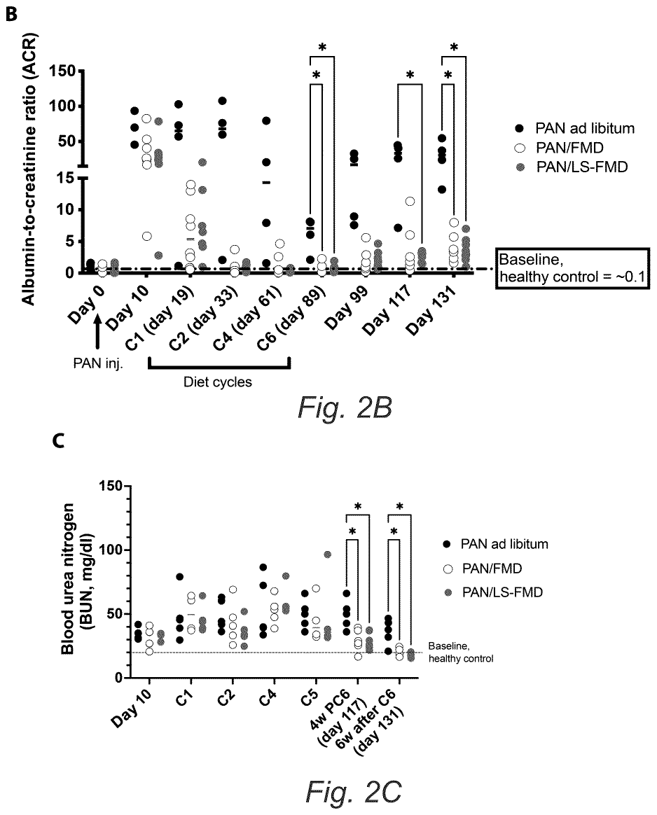

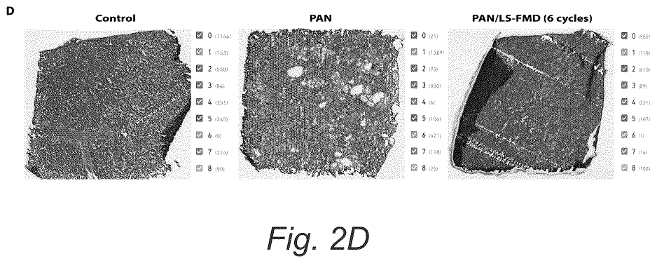

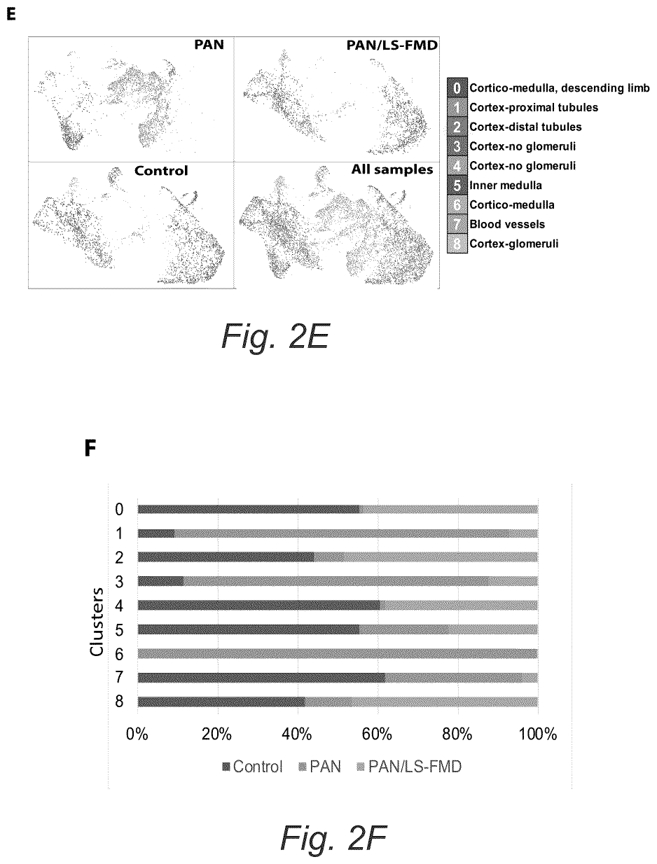

To evaluate the renoprotection efficacy, PAN rats were treated with six FMD or LS-FMD cycles, each lasting 4 days, starting on day 14 after PAN injection, after the peak of acute injury. Each cycle was followed by a week of refeeding ad libitum (FIG. 2A). Healthy rats, PAN rats treated with six cycles of the regular FMD, and PAN rats fed ad libitum were used as controls. Rats treated with either LS-FMD or FMD showed a significant reduction (P<0.05) in ACR and blood urea nitrogen (BUN) at 4 and 6 weeks after six diet cycles (FIGS. 2, B and C) compared with rats fed ad libitum. In addition to observing improvement in these physiological parameters, histological evaluation confirmed that rats treated with LS-FMD or FMD showed a significant reduction (P<0.05) in tubular injury, after one cycle and after six cycles at end point, compared with ad libitum PAN rats.

To understand the molecular changes induced by the diet, spatial transcriptomics [ST; Visium 10× Genomics platform; (32-34); fig. S8] was performed. For the ST, rats treated with LS-FMD was used to focus on a kidney diet better suited for patients with kidney disease. Spatial maps of gene expression of kidney sections were generated from a healthy rat (control), a PAN-treated rat (PAN), and an age-matched PAN-treated rat after six cycles of LS-FMD (PAN/LS-FMD).

Kidney structures with representative gene expression are shown in the supplemental material for Villani et al. (Sci. Transl. Med., 2024). Established renal markers were used to match transcriptionally defined clusters to specific histological structures (35, 36). Samples were first analyzed individually to identify spatially distinct transcriptomic regions for each sample. Seven different clusters were identified in the control and PAN groups, whereas six clusters were detected in the PAN/LS-FMD sample; this clustering is also represented in the Uniform Manifold Approximation and Projection (UMAP) visualization for each sample. No cluster corresponding to blood vessels was detected in the PAN/LS-FMD samples, because this specific section did not include any large vessels, which are instead present in the other samples. The 10 most highly expressed genes for each identified cluster are reported in the supplemental material for Villani et al. (Sci. Transl. Med., 2024).

To investigate differences between PAN/LS-FMD and PAN rats, using a healthy kidney as a control reference, integrative analysis of all three samples was performed; nine clusters were identified by unsupervised clustering (FIG. 2D). The histological structures associated with each cluster, together with the top 10 expressed genes in each cluster, are reported in the supplemental material for Villani et al. (Sci. Transl. Med., 2024).

A UMAP representation showed that the PAN rat subjected to multiple cycles of LS-FMD had a spatial transcriptional profile comparable to those of healthy controls at the end point (FIGS. 2, E and F), whereas the PAN sample showed a distinctly different gene expression pattern. Major gene expression changes were observed in clusters 1, 3 and 6, as a result of PAN injury (FIG. 2F); these potentially represent two distinct populations of proximal tubular cells (clusters 1 and 3) and a cortico-medullary (outer medulla) region in the PAN rat (cluster 6). Clusters 0, 2, and 4 (mainly found in healthy and PAN/LS-FMD) represented, respectively, a cortico-medullary region/descending limb, cortex/distal tubules, and cortex/proximal tubules. The inner medulla (cluster 5) did not seem to be affected by the PAN treatment or dietary intervention. Cluster 7, which identified blood vessels, and cluster 8, predominantly present in healthy and PAN/LS-FMD, corresponded to the glomerular compartment (FIG. 2F). ST analysis allowed us to study transcriptional differences in histologically similar regions in healthy and LS-FMD-treated versus PAN rats; for example, the UMAP split by sample showed a molecular shift of clusters 0 and 6, respectively, which represented the cortico-medullary region in each sample. Reactome analysis confirmed enrichment of pathways related to the metabolism of lipids and biological oxidation in healthy and LS-FMD-treated rats, whereas, in PAN-treated rats, this region displayed enrichment of immune system pathways. These data indicate that, in nephrotic rats, multiple cycles of LS-FMD improved kidney function long term and restored the transcriptional profile of the major kidney structures, resulting in patterns similar to those observed in a healthy rat.

### Cycles of LS-FMD Protect Glomerular Structure and Function in PAN-Treated Rats

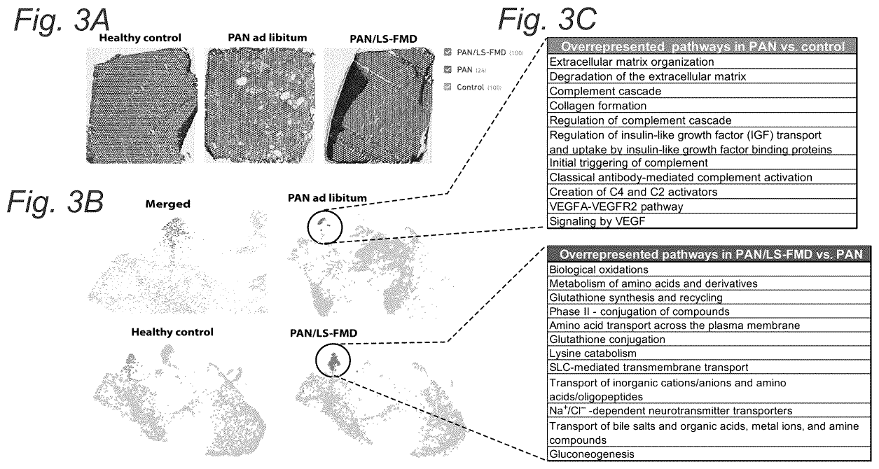

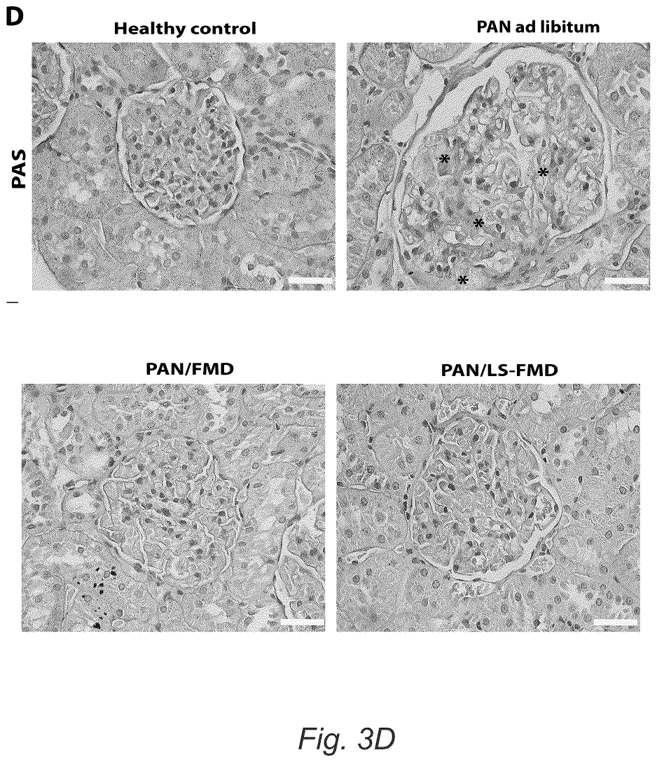

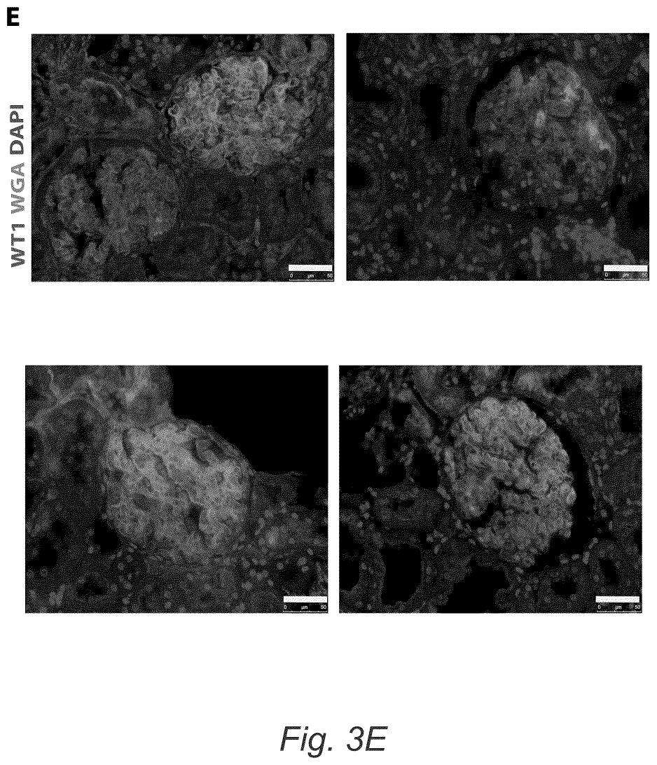

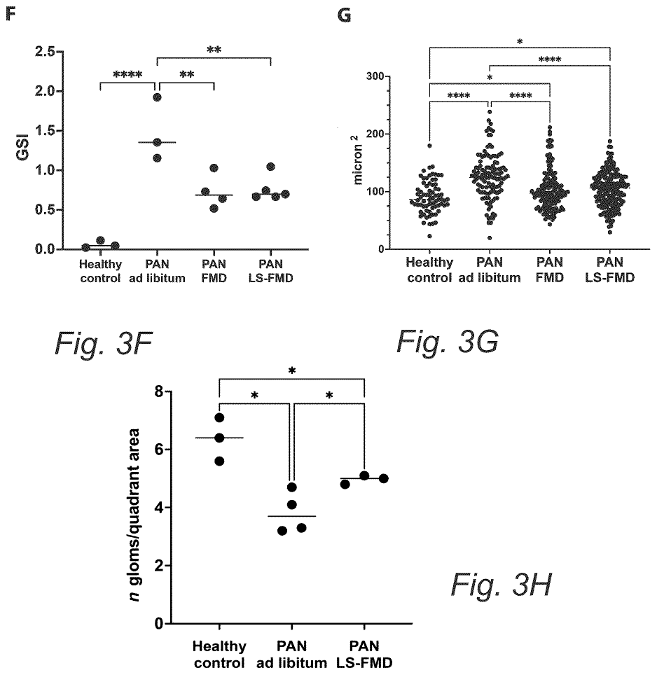

Because glomerular damage is the ultimate cause of the injury in the PAN model (and of other CKDs), the ST analysis focused, specifically on glomeruli. ST spots expressing four known podocyte markers (WT1, Nphs1, Nphs2, and Robo2; FIG. 3A) were then selected. The resulting spots overlapped with cluster 8 from unsupervised clustering (FIGS. 2E and 3, A and B). The healthy and the PAN/LS-FMD kidney sections appeared to show a higher number of glomerular spots compared with the PAN sample (FIG. 3A). Differential gene expression (DGE) analysis identified pathways involved in collagen formation, extracellular matrix degradation, and regulation of insulin-like growth factor (IGF) binding proteins as up-regulated in PAN glomeruli compared with those in healthy controls, whereas up-regulation of metabolic pathways, such as biological oxidation, amino acid metabolism and gluconeogenesis, glutathione synthesis, and recycling, was observed in the LS-FMD-treated sample compared with that in the PAN-treated rats (FIG. 3C). It was shown that podocytes primarily use fatty acid oxidation and strongly depend on anaerobic glycolysis (37). Therefore, a shift toward increased biological oxidation induced by the diet might contribute to reestablishing metabolic homeostasis in these cells. Moreover, the metabolism of amino acids, coupled with gluconeogenesis, are expected responses to fasting to meet energy needs. Glutathione treatment was shown to decrease oxidative stress, decrease apoptosis, and increase expression of podocyte and tubular markers in human kidney organoids (38), suggesting that the process of glutathione synthesis and recycling induced by the LS-FMD might also contribute to podocyte preservation. Up-regulation of the complement cascade also emerged in PAN group glomeruli only. Activation of complement components has been described in murine models of nephrosis (39) and in humans with focal segmental glomerulosclerosis (40).

The improvements in the glomerular specific transcriptomic maps detected by ST were corroborated by histological evidence. Glomeruli from rats that received either LS-FMD or FMD showed normal glomerular structure, with no apparent sclerosis and less glomerular hypertrophy compared with those from the PAN-treated rats (FIG. 3, D to G). Glomerular sclerosis was significantly (P<0.05) lower after one cycle of either LS-FMD or FMD compared with that in PAN rats, and this effect was sustained long term (FIG. 3F). This was coupled with overall higher expression of NEPHRIN in glomeruli from rats treated with either diet at long term. In addition, the average number of glomeruli per section was significantly (P<0.05) higher in rats that received LS-FMD compared with that in PAN ad libitum rats (FIG. 3H).

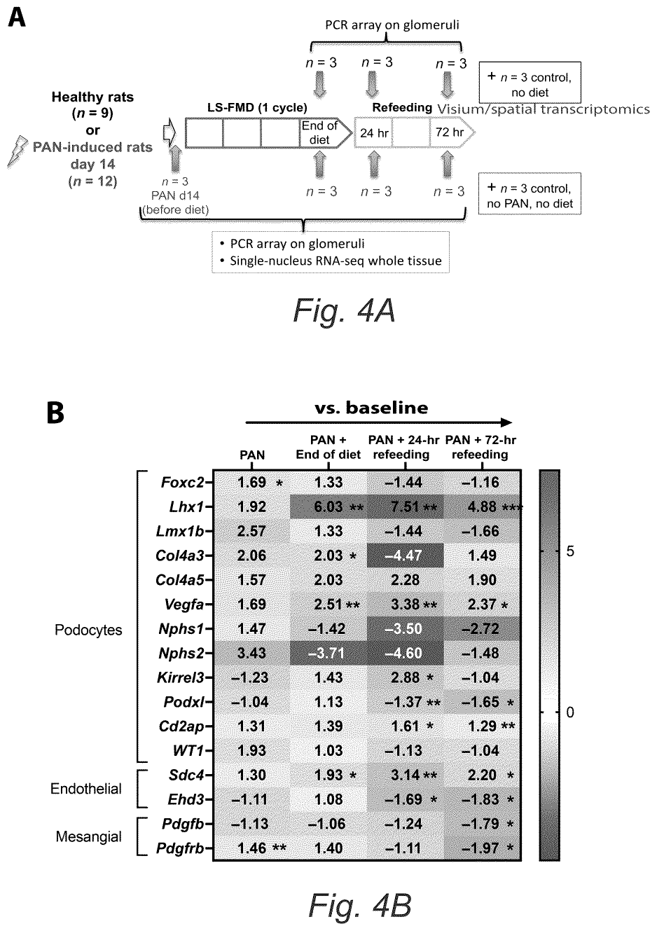

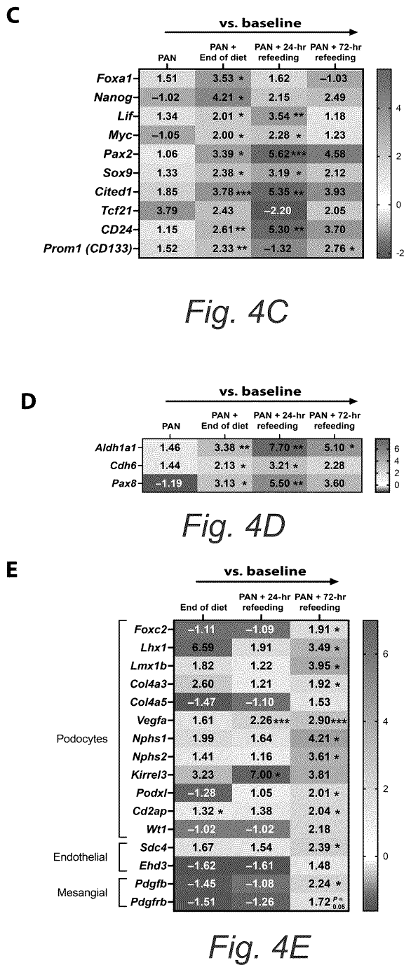

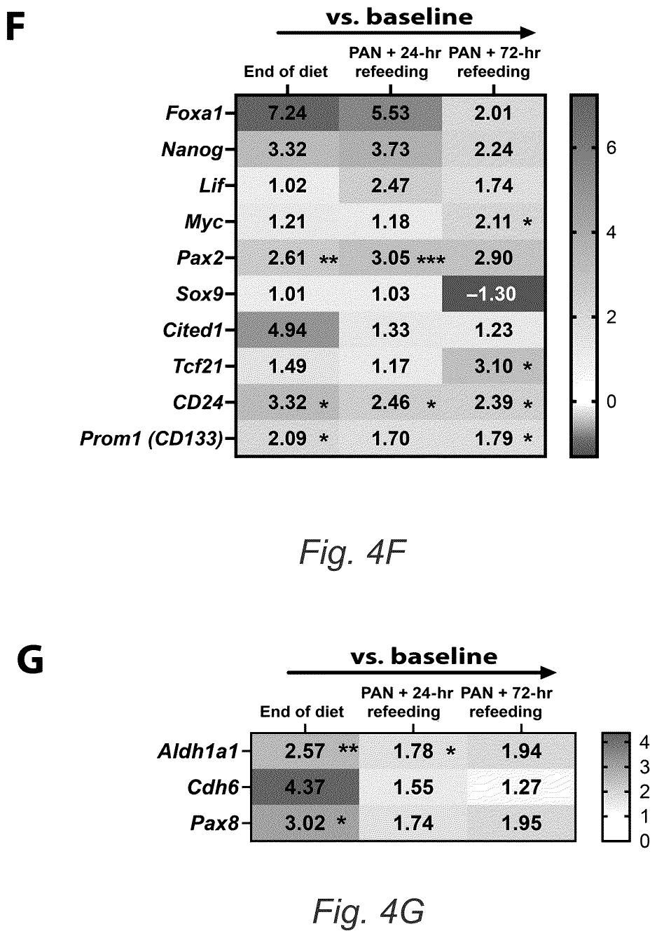

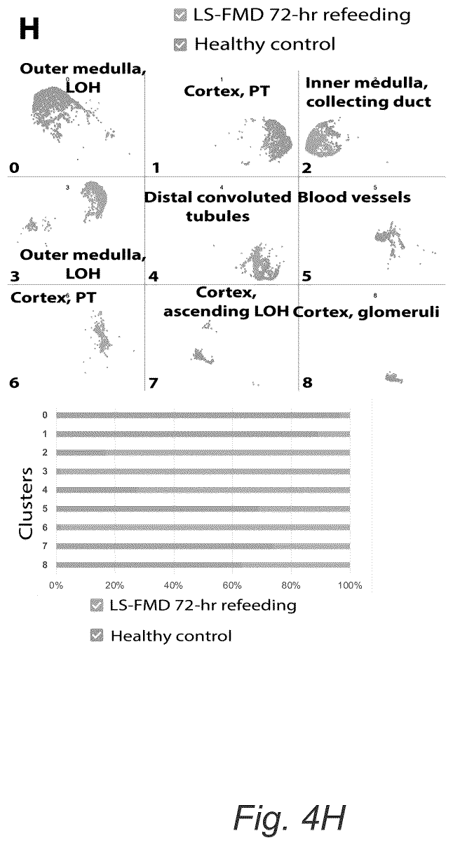

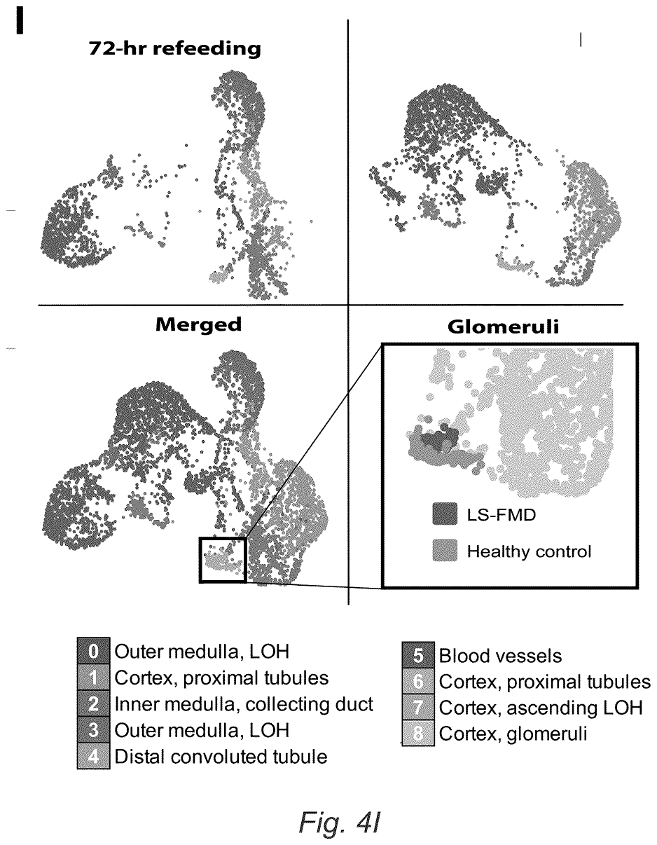

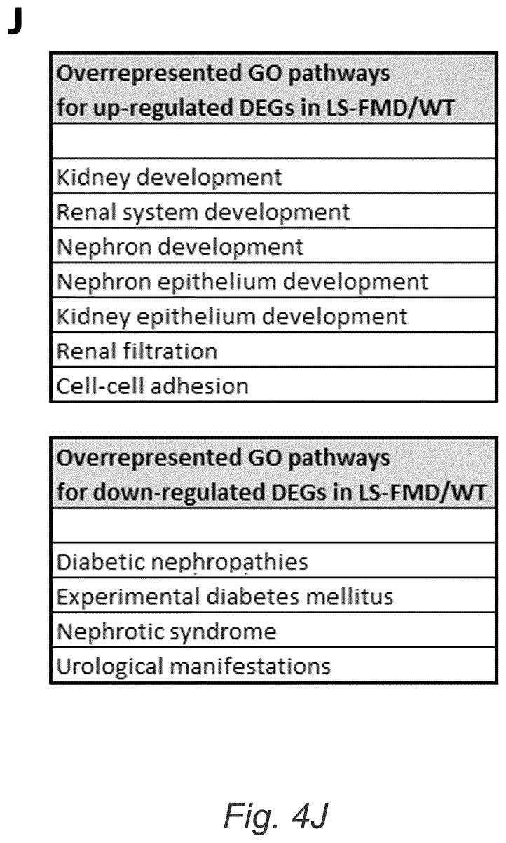

To better understand the short-term molecular changes occurring in the glomeruli during LS-FMD treatment, PAN rats were subjected to a single cycle of LS-FMD (FIG. 4A) and analyzed glomerular gene expression using a custom quantitative polymerase chain reaction (qPCR) array at the end of the diet period (EoD) and at 24 and 72 hours after refeeding.

Major gene changes were observed at the EoD and 24 hours after refeeding, as shown in the heatmap and in DGE analysis. It is evident that the diet reinforced the glomerular cell-specific gene expression profile, including those of podocytes, GECs, and mesangial cells (with up-regulation of genes like Lxh1, Col4a5, Vegfa, Kirrel3, and Sdc4; FIG. 4B). The FMD induced up-regulation of not only genes associated with glomerular development (like Foxa1, Lif Pax2, Cited1, and Sox9) but also of pluripotent embryonic stem cell and organogenesis-associated genes Myc and Nanog (41), which were down-regulated in the PAN glomeruli and up-regulated at the end of LS-FMD. The up-regulation of parietal epithelial cell (PEC; considered podocyte precursors) genes (such as Aldh1a, Cdh6, and Pax8) at the end of LS-FMD and 24 hours after refeeding is also noted, showing an expression pattern matching that of embryonic stem cell-associated genes (FIGS. 4, C and D) (42-45). Increased protein expression of renal progenitor lineage markers, such as Pax2, Aldh1a1, and Cited1, was also detected in isolated glomeruli from diet-treated rats compared with those from the controls. These data suggest that the LS-FMD not only reversed gene expression changes induced by the damage but also appears to stimulate a “glomerular regenerative/repair” process by potentially activating cellular reprogramming pathways.

Using the same qPCR array, one cycle of LS-FMD and refeeding in glomeruli from healthy rats were also evaluated to investigate the effect of the diet independent of damage. By comparing gene expression of glomeruli from age-matched healthy rats (no damage, fed ad libitum), it was confirmed that the LS-FMD activated developmental kidney and stem cell genes, as well as PEC-specific genes, with the strongest effects occurring at the end of the LS-FMD and at 72 hours after refeeding (FIG. 4, E to G).

To expand the analysis to other kidney structures and to other gene changes, in addition to the ones present in the custom qPCR array, ST on a kidney section of a healthy rat treated was also performed with one cycle of LS-FMD and 72 hours after refeeding and compared the data with those of a rat fed ad libitum. Via integration analysis, nine clusters (FIGS. 4, H, and I) were identified. Whereas the composition of most clusters was shared between the control and LS-FMD samples, some clusters were predominantly represented by one of the two samples (clusters 0 and 3, identifying the medullary region; and clusters 1 and 6, identifying the proximal tubules). DGE and Reactome analysis performed between clusters 0 and 3 revealed overexpression of genes involved mainly in the lipid metabolism in the LS-FMD sample. Similar results were also evident by DGE analysis of clusters 1 and 6, suggesting that, in the kidney tubular components, the effects of the diet during the first days are mainly characterized by metabolic changes and lower oxidative stress damage. The glomerular cluster was again characterized by enrichment of Gene Ontology (GO) sets related to developmental pathways such as kidney development, renal system and nephron development, nephron epithelium development, and renal filtration in the LS-FMD-treated rats (FIG. 4J). In addition, the LS-FMD sample in clusters 2 (inner medulla/collecting duct), 4 (distal convoluted tubules), 5 (blood vessels), and 7 [ascending loop of Henle (LOH)] also exhibited overrepresented GO terms related to kidney development, metanephric duct development, and kidney cell development processes, thus further confirming that even in physiological conditions, the diet can stimulate kidney regeneration.

### LS-FMD Activates Endogenous Podocyte Regeneration and Stabilizes the Podocyte Cell Cycle

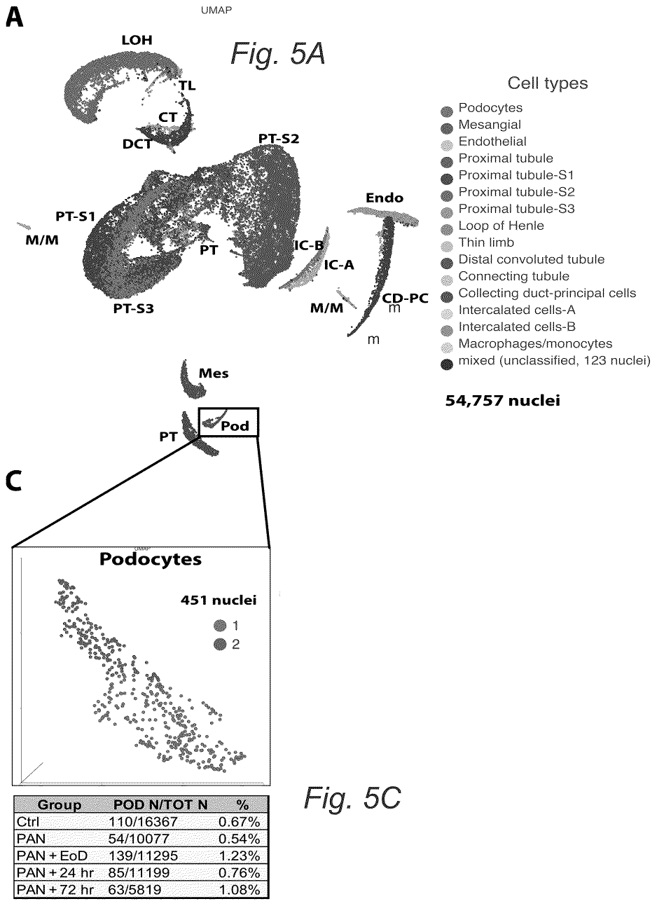

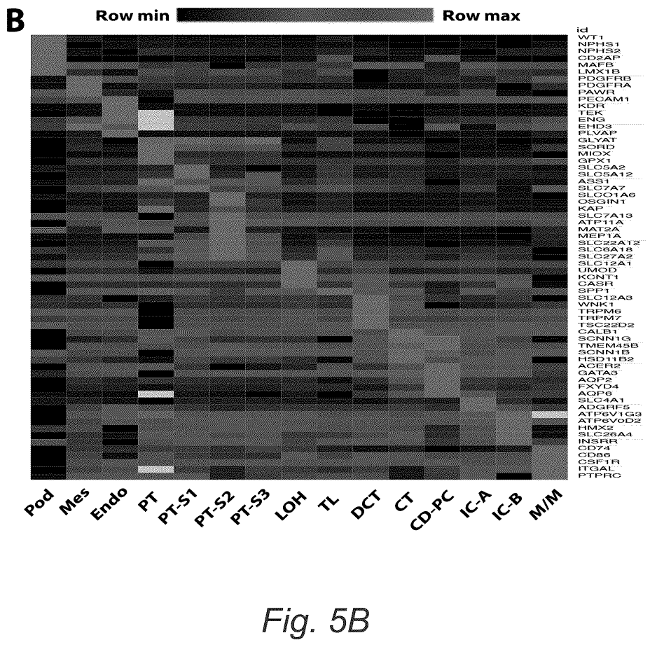

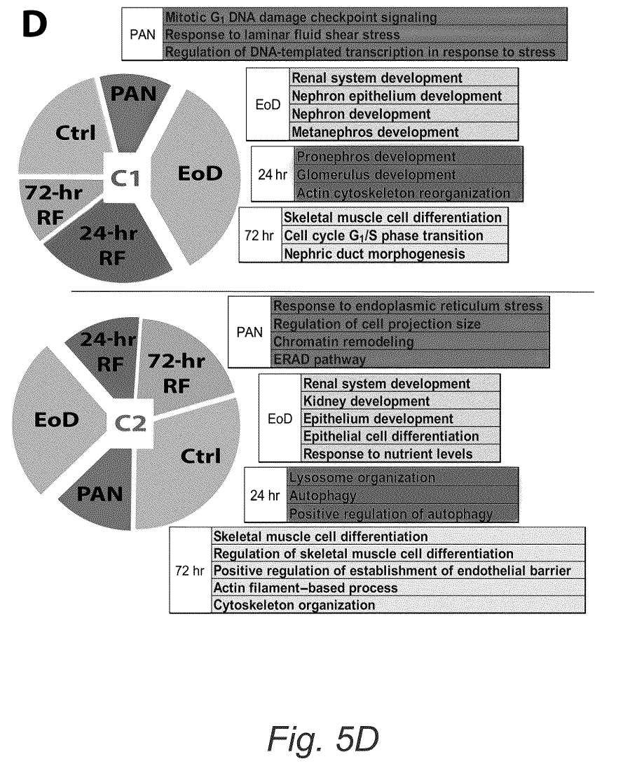

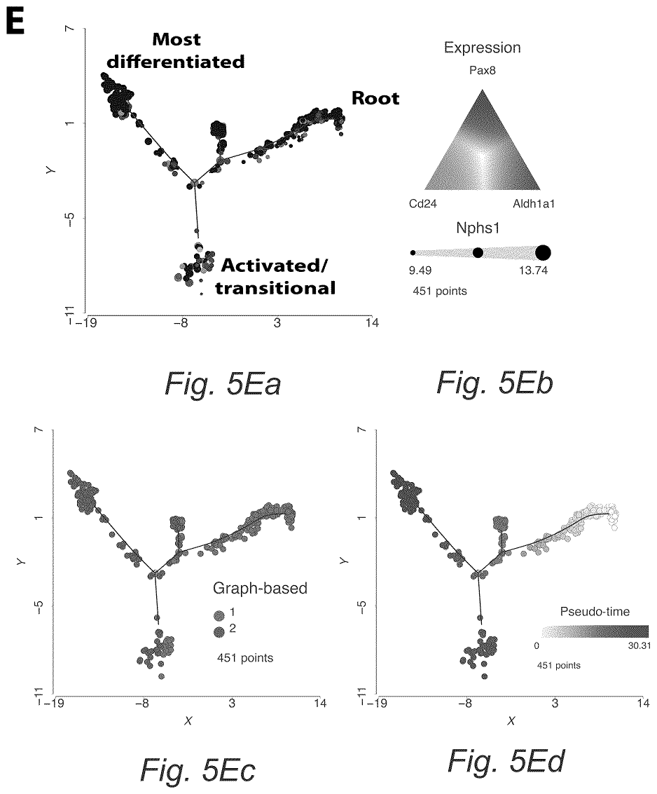

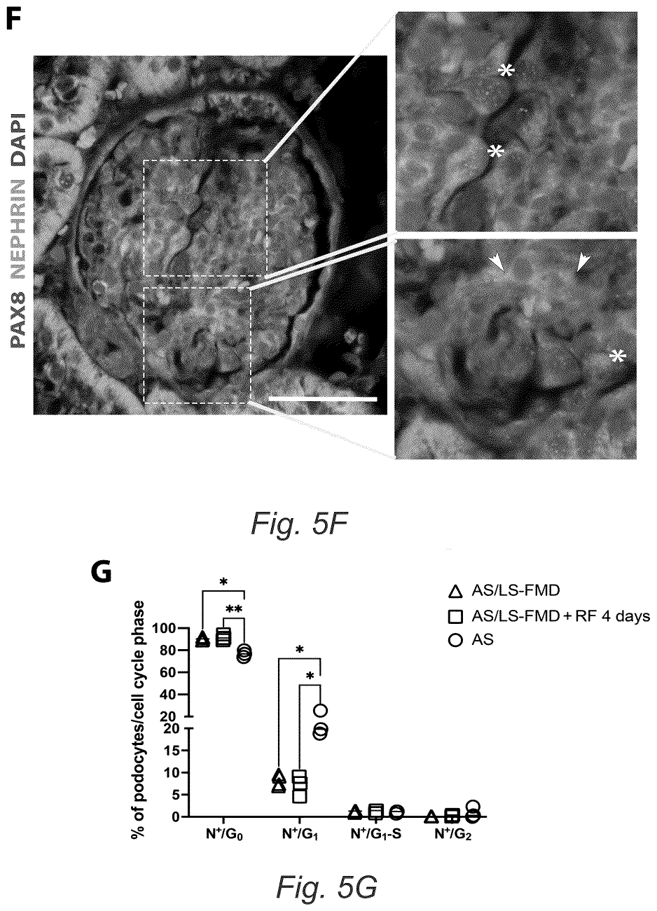

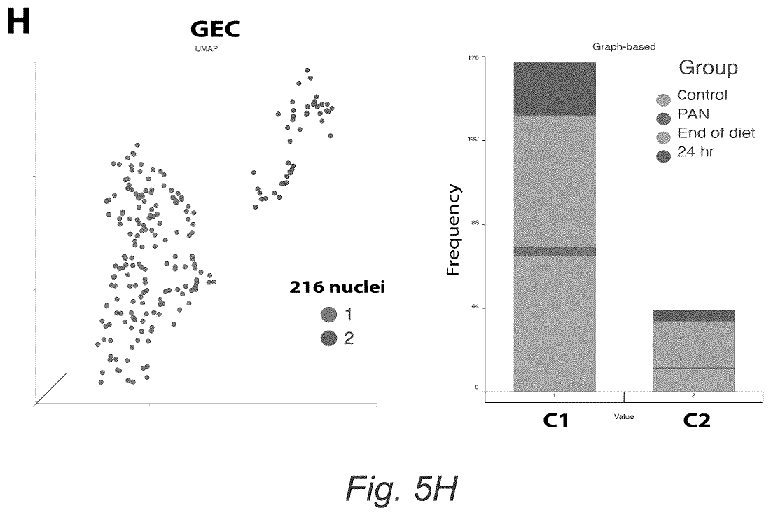

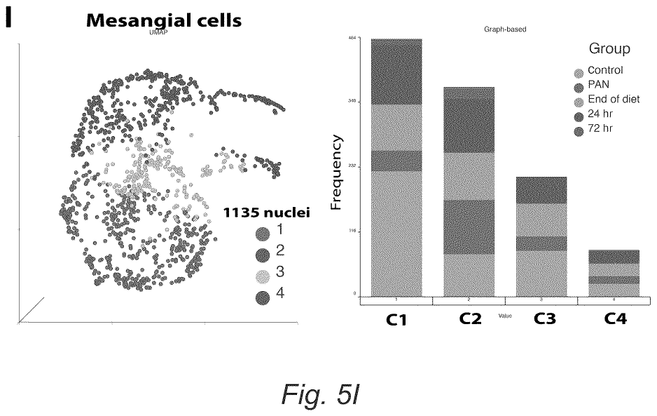

To investigate the potential of the LS-FMD to regulate cell populations within the kidney, single-nucleus RNA sequencing (snRNA-seq) was performed in PAN-treated rats ad libitum and PAN-treated rats subjected to one cycle of LS-FMD and analyzed at EoD, 24 hours, and 72 hours after refeeding (n=3 per group). Healthy rats fed ad libitum were used as a control (n=4). After filtering the nuclei for read counts (500 to 80,000), detected features (100 to 8000), and mitochondrial counts (<5%), a total of 54,757 sequenced nuclei from all the samples were analyzed. The different kidney cell types were classified using a set of specific markers as published (35, 36, 46, 47). To reduce batch effects, the classification was performed for each individual sample separately. The UMAP of the integrated samples identified 15 distinct clusters (FIG. 5A). A group of nuclei (n=123) was unclassified. A split-by-sample view of the populations' distribution is reported in the supplemental material for Villani et al. (Sci. Transl. Med., 2024), and a gene expression heatmap of kidney lineage-specific markers validated cell identity annotation within the dataset (FIG. 5B). GECs were classified as a subpopulation of the endothelial cell cluster in FIG. 5A, based on the expression of Ehd3, Plvap, and Hecw2.

Given the focus on podocytes, this population was filtered by the expression of WT1, Nphs1, and Nphs2 and re-clustered; 451 nuclei from all five groups were assigned a podocyte identity and distributed in two distinct subclusters, C1 and C2 (FIG. 5C).

The integration analysis of all samples shows an increase in the percentage of podocyte nuclei in the LS-FMD groups, particularly in the EoD group (almost double compared with the same population in the PAN or control groups; see table in FIG. 5C). The contribution of podocyte nuclei from the different experimental groups relative to the two subclusters is shown in FIG. 5D. Enrichment analysis between the different groups within C1 or C2 showed that the PAN-treated podocytes present changes in cell cycle (G₁ DNA damage checkpoint signaling) and metabolic pathways in response to stress in C1 and C2, respectively. In contrast, the podocytes of the EoD group in C1 presented with GO sets enrichment of renal developmental biological processes (metanephros development, renal system development, etc.). These pathways were also slightly enriched in the EoD group in C2, which was mostly characterized by up-regulation of GO sets of mature podocyte phenotype and differentiation. The stabilization of the podocyte gene signature involving pathways like actin filament-based processes, cytoskeleton organization, regulation of Wnt signaling, and muscle cell differentiation was also observed at 24 and 72 hours after refeeding, both in the C1 and C2 podocyte clusters. In the EoD group, C1 nuclei also showed higher expression of Pax8, CD24, and Aldh1a1, which are PEC-specific genes (42-45) (not expressed in fully mature podocytes), but no significant expression (P>0.05) of CD44, recognized to be a marker of disease-activated PECs (48, 49), indicating the presence of a putative population of cells expressing both PEC and podocyte markers.

Dynamic changes in gene expression in podocytes were investigated via trajectory analysis (FIG. 5E), revealing five different states, with states 1, 2, and 3 highly represented in the EoD group. Pax8-, CD24-, and Aldh1a1-expressing cells were mainly present in the EoD and 24-hours-after-refeeding groups, in agreement with the analyses shown in previous figures, with Pax8 and CD24 more evenly distributed along the five states and Aldh1a1 mainly in states 1 and 3, suggesting that these two states might represent regenerative/developmental podocyte features.

The overlay of the clusters along the trajectory identifies a root, colocalizing with state 1 and an intermediate state (3) represented by potentially activated podocyte progenitor cells. Because these cells were filtered on the basis of the expression of terminal differentiation markers for podocytes, Nphs1 expression was looked at along the trajectory. Nuclei belonging to states 4 and 5, which also present with fewer progenitor-like genes, have the highest Nphs1 expression (FIG. 5E), suggesting that the gene expression of these later stages identified mature podocytes, thus identifying a temporal progression of podocytes from development to differentiation through the course of one LS-FMD cycle. Histologically, the presence of cells co-expressing PAX8 and NEPHRIN in the LS-FMD-treated rats was confirmed in the middle of the tuft (FIG. 5F). These double-positive cells are distinct from NEPHRIN⁺ only cells and PAX8⁺ only cells lining the capsule of Bowman, which identify fully matured podocytes and PECs, respectively. These data suggest the presence of a putative podocyte precursor and its trans-differentiation and migration within the glomerular space. These data are in agreement with the data analysis above described for C1 and C2, confirming that the diet can activate podocyte precursors and stabilize mature podocytes.

One of the most important features of mature functional podocytes is their maintenance of the quiescent phase or G₀ state of the cell cycle: A nonproliferative state is essential for podocytes to be able to perform their ultrafiltration activity (50). During kidney disease, podocytes tend to reenter the cell cycle, which results in detachment from the glomerular basement membrane and loss in the urine (51-55). The qPCR array analysis showed an increased gene expression of specific cyclin-dependent kinase inhibitors such as Cdkn1c (p57), Cdkn1a (p21), and Cdkn1b (p27) at the end of LS-FMD and upon refeeding in healthy conditions. After injury induction, a significant up-regulation (P<0.05) of cyclins Ccne1 and Ccna2 was detected, which are involved in the mitotic phase of the cell cycle. This up-regulation was reversed by LS-FMD. One cycle was sufficient to induce a significant down-regulation (P<0.05) of these cyclins in injured glomeruli, with an increase in the cyclin-dependent kinase inhibitors Cdkn1a (p21) and Cdkn1b (p27). The increase in p21 and p27 proteins after the diet as well was confirmed. In addition, the snRNA-seq showed that, in the LS-FMD groups, there was a regulation of the cell cycle compared with the PAN-damaged podocytes, in which an activation of the G₁-S phase transition was also evident.

Using an Alport syndrome mouse CKD model carrying the fluorescent ubiquitination-based cell cycle indicator (FUCCI) specifically in podocytes (POD-FUCCI) (56), the LS-FMD can influence the cell cycle in podocytes was evaluated in vivo. It was decided to use the Alport mouse model for cell cycle studies because of its ability to generate accurate and reproducible results, rather than relying on fluorescent antibody-based staining to track cell cycle changes if PAN rats were used. 5-month-old Alport syndrome POD-FUCCI mice were subjected to one cycle of LS-FMD and analyzed the percentage of podocytes (NEPHRIN⁺ cells) in different stages of the cell cycle by flow cytometry. The gating strategy is shown in the supplemental material for Villani et al. (Sci. Transl. Med., 2024). A group of mice did not receive the diet and was used as an ad libitum control. Mice that received the LS-FMD displayed a greater percentage of podocytes in the Go phase (NEPHRIN-positive, no FUCCI signal detected) and a lesser percentage of podocytes in the G₁ phase of the cell cycle compared with AS mice fed ad libitum (FIG. 5G), both at the EoD and after refeeding. This result suggests that one cycle of LS-FMD is sufficient to reduce cell cycle entry and stabilize podocytes in the Go phase in a model of chronic kidney injury.

### LS-FMD Activates Regenerative Pathways in Multiple Kidney Cell Types

In addition to analyzing the effect of LS-FMD on podocytes, changes in gene expression were also investigated during and after the LS-FMD in different kidney compartments via snRNA-seq. First, GECs and mesangial cells were filtered and re-clustered because both are part of the glomerulus. Two subclusters for GECs and four subclusters for mesangial cells (FIGS. 5, H, and I) were obtained. The distribution of nuclei per group and clusters is shown in the bar charts. GECs in the samples were not detected at 72 hours after refeeding, probably because of a lower number of cells that passed the quality control for this smaller group. Whereas a lower number of glomerular endothelial cells (GECs) was also detected in the PAN group, this reduction was presumed to be biologically driven, as indicated by the total number of nuclei per group (FIG. 5C). In the subcluster C1 of the GECs in the EoD group, there was an up-regulation of pathways related to the circulatory system and blood vessel morphogenesis when compared with the other groups.

All the proximal tubule populations were then filtered into one integrated group; unsupervised clustering produced 13 distinct clusters in this group. A split-by-group UMAP representation indicated gene expression changes, encompassing all segments of the proximal tubules. As shown in the supplemental material for Villani et al. (Sci. Transl. Med., 2024), three clusters (6, 10, and 13) were mostly or exclusively represented by nuclei of the EoD group. Pathway analysis showed enrichment of nucleotide metabolic processes, lipid metabolism processes, and pH regulation (one of the primary functions of proximal tubules), indicating a switch in metabolic state of these PT cells, which belong to the Si segment, during LS-FMD. Both clusters 10 and 13, however, displayed enrichment of GO sets relative to epithelial cell development, tube morphogenesis and development; regulation of cell proliferation, migration, and adhesion, indicating a developmental identity and suggesting ongoing cellular regeneration in the segments S2 and S3 of the PT induced by the LS-FMD compared with the PAN-damaged tubule. A similar regenerative cluster (cluster 8) exhibiting developmental features was also identified within another anatomical structure of the kidney, the loop of Henle (LOH), in the EoD group. In contrast, no relevant differences were detected in the connecting tubule cell fraction, unlike those observed in glomerular, tubular, and LOH cells. Up-regulation of Zbtb16 was observed in most of the nuclei from the LS-FMD group, including podocytes and proximal tubular cells, suggesting a possible role of this transcription factor in response to fasting. These snRNA-seq data indicate that the LS-FMD followed by refeeding can activate developmental, regenerative, and metabolic pathways in multiple kidney structures, which may underlie renoprotection, regeneration and repair both functionally and structurally.

### FMD Reduces Disease Severity in Murine Lupus Nephritis

To expand the potential clinical translatability of the fasting-mimicking diet (FMD) approach for treating chronic kidney disease (CKD), mice that naturally develop lupus nephritis were utilized. One-month-old female MRL/lpr mice, which spontaneously develop lupus, were subjected to weekly FMD cycles or maintained on an ad libitum diet for up to 15 weeks of follow-up. Body weight decreased during each fasting cycle but fully recovered during the subsequent refeeding period. The severity of skin lesions progressively increased in ad libitum-fed mice but remained unchanged in FMD-treated mice. Albumin-to-creatinine ratio (ACR) and blood urea nitrogen (BUN) levels were significantly lower (P<0.05) in the FMD group, correlating with improved renal histological scores. Furthermore, FMD cycles reduced anti-double-stranded DNA autoantibody levels and glomerular immunoglobulin G deposition, indicating potential anti-inflammatory and reno-protective effects of the intervention. Overall, these data indicate that cycles of FMD are safe and can effectively reduce or delay the progression of lupus nephritis in mice.

FMD Cycles Ameliorate Kidney and Endothelial Function, Reduce Inflammation, and Increase Circulating Kidney Progenitor Cells in Patients with CKD

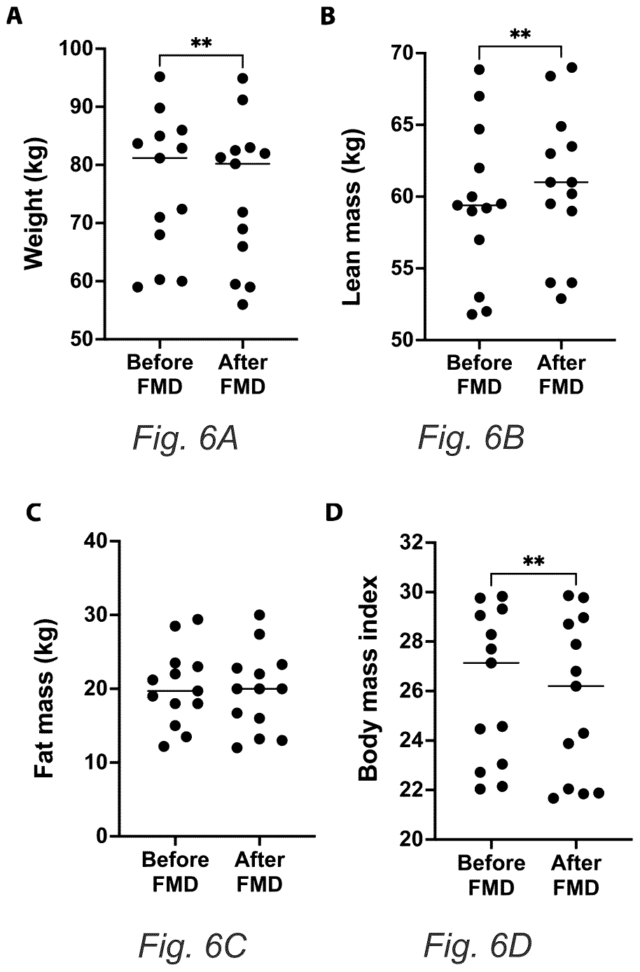

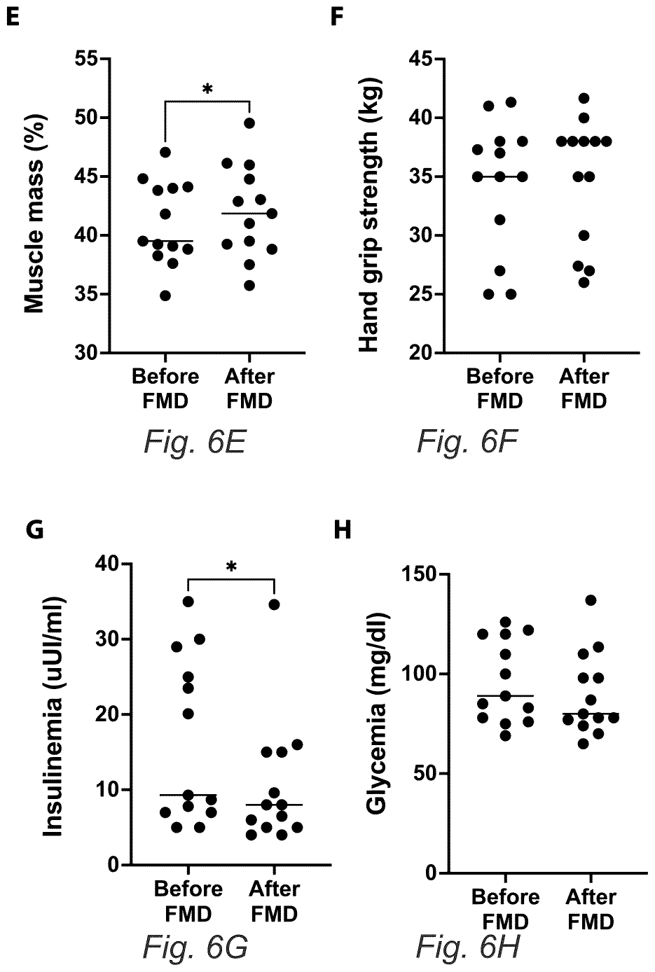

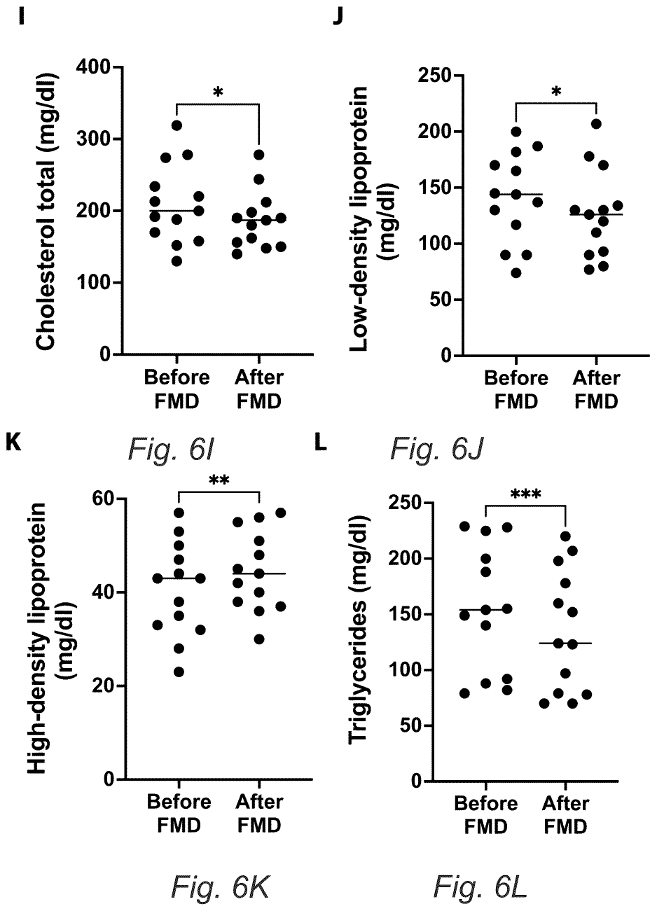

To evaluate whether FMD (LS-FMD has not yet been developed for human application) is nephroprotective in humans, a pilot clinical study including 13 patients with CKD (stage 3) was performed. Baseline characteristics and specific diagnoses of the cohort are reported in Table 1. Seven patients were subjected to three cycles of FMD over a 3-month period and were monitored before and after the FMD treatment, whereas six patients were assigned to a control group. After 3 months, the control group was crossed over to also receive three FMD cycles, for a total of 13 patients; measurements were made at baseline and after undergoing three FMD cycles. One hundred percent compliance with the dietary intervention was reported with minor side effects, such as headache, for four patients, which did not impede continuation of the dietary regimen. Patients were not receiving concurrent immunosuppressive therapy during the study, but all patients were taking angiotensin-converting enzyme (ACE) inhibitors and/or angiotensin receptor blockers. Results showed a significant (P<0.05) but mild weight loss, increased lean mass and muscle mass (based on BIA analysis), reduced BMI, and insulinemia, whereas fat mass, hand grip strength, and glycemia remained unchanged (FIG. 6, A to H); all values before and after treatment were within the normal range for all patients. Moreover, FMD intervention resulted in significantly (P<0.05) improved lipid panel markers (FIG. 6, I to L), and the patients' overall well-being and health state was confirmed through cognitive and psychological assessment for depression and state anxiety (57), which were both improved after FMD application. A similar trend, although not reaching significance (P>0.05) in all measurements likely because of the low number of patients per group, was observed when comparing the FMD group with the control group. In the control group, no significant changes (P>0.05) were detected after 3 months on a free diet; however, most of the parameters significantly (P<0.05) improved after completing the FMD cycles.

TABLE 1
Patient information. The table reports the gender, age,
and specific renal pathology of each enrolled patient.

| Patient | Gender | Age | Pathology |
|---|---|---|---|
| 1 | M | 53 | IgA nephropathy |
| 2 | M | 63 | IgA nephropathy |
| 3 | F | 59 | Membranoproliferative |
| | | | glomerulonephritis |
| 4 | M | 55 | Membranous nephropathy |
| 5 | M | 62 | IgA nephropathy |
| 6 | M | 40 | IgA nephropathy |
| 7 | F | 38 | Membranous nephropathy |
| 8 | M | 47 | Focal segmental glomerulosclerosis |
| 9 | F | 38 | Minimal change disease |
| 10 | F | 49 | Focal segmental glomerulosclerosis |
| 11 | M | 41 | Membranous nephropathy |
| 12 | M | 40 | Focal segmental glomerulosclerosis |
| 13 | M | 44 | Minimal change disease |

IgA, immunoglobulin A; F, female; M, male.

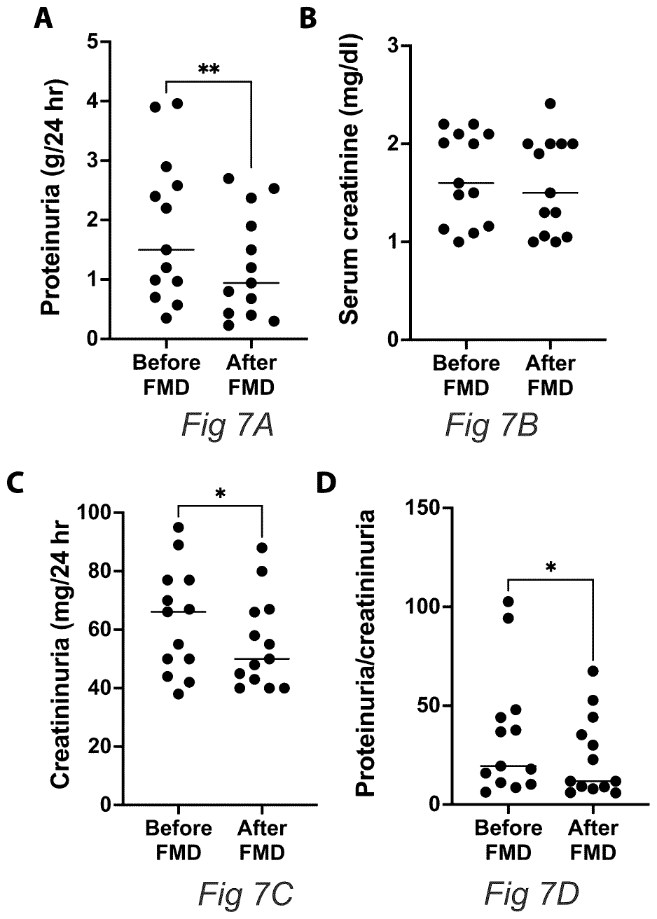

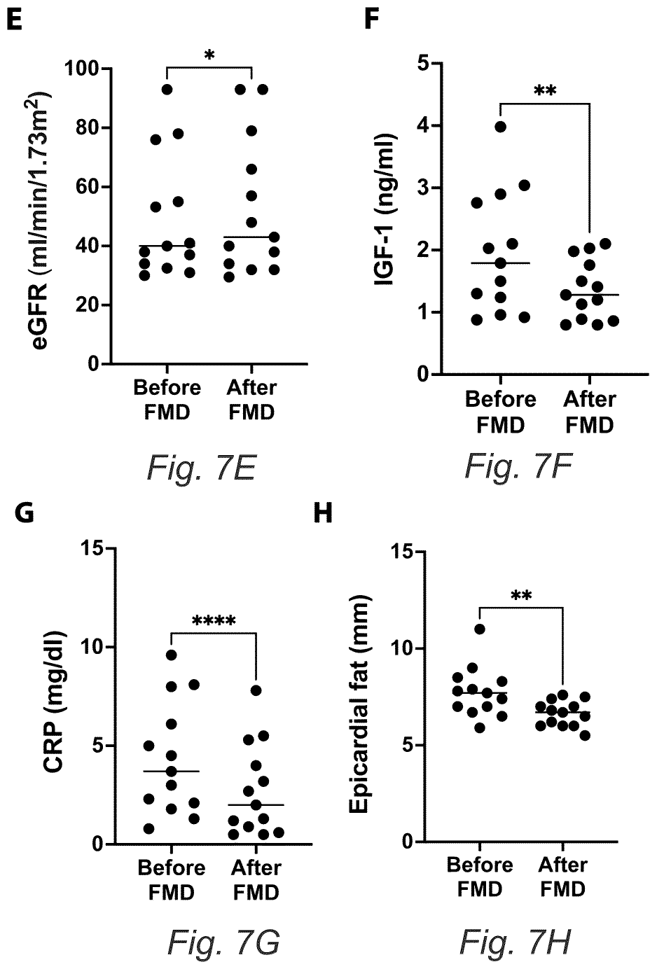

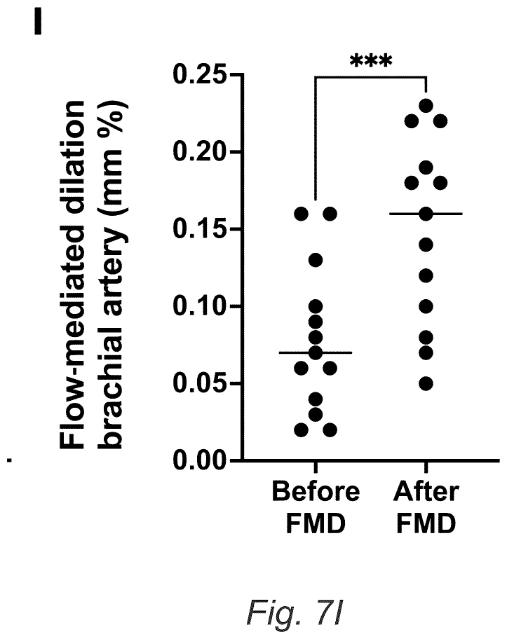

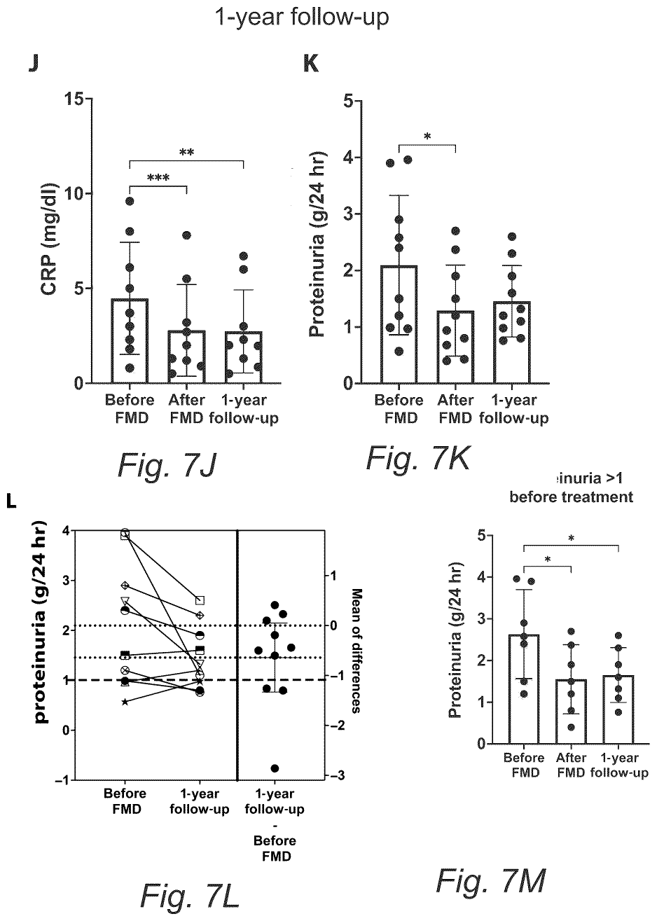

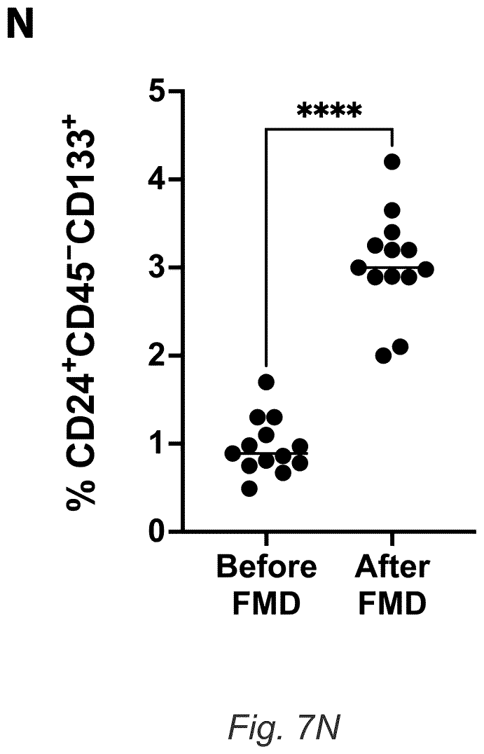

Several parameters associated with kidney function, endothelial integrity, and inflammation were analyzed. A significant reduction in proteinuria (P<0.05) was observed following three cycles of the fasting-mimicking diet (FMD) (FIG. 7A), while serum creatinine levels remained unchanged (FIG. 7B). Creatinine excretion was also significantly reduced (P<0.05) after three FMDs, suggesting increased muscle anabolism (FIG. 7C). In addition, the protein-to-creatinine ratio and estimated glomerular filtration rate (eGFR) were significantly (P<0.05) improved in patients after FMD cycles (FIGS. 7, D and E).

Consistent with the cell protective and anti-inflammatory properties of FMD treatment, significantly lower (P<0.05) IGF-1 and C-reactive protein (FIGS. 7, F and G) were observed. Of interest, epicardial fat was reduced, and the flow-mediated dilation of the brachial artery, an index of endothelial function in patients with CKD, was significantly improved (P<0.05) after the FMD treatment (FIGS. 7, H and I), thus highlighting the potential of FMD to improve both kidney and vascular/endothelial function in patients with kidney disease. These data are also represented before and after FMD per each individual patient.

To assess the durability of the fasting-mimicking diet (FMD) effects, follow-up data was obtained from a routine clinical visit conducted one year after completion of treatment in 10 out of the 13 enrolled patients. Proteinuria, serum creatinine, and other parameters were assessed (FIG. 7, J to M). After 1 year, C-reactive protein was still significantly (P<0.05) lower (FIG. 7J), and most patients displayed a continued reduced proteinuria compared with baseline (FIGS. 7, K and L). These patients were not advised to follow any other dietary regimen or continued cycles of FMD. When analyzing only the subgroup of patients who exhibited proteinuria greater than 1 g per 24 hours before treatment [n=7, a prognostic threshold indicative of disease severity (58)], a significant reduction in proteinuria (P<0.05) was observed at the one-year follow-up following completion of the fasting-mimicking diet (FMD) treatment (FIG. 7M).

FMD cycles have been shown to stimulate the activation and proliferation of hematopoietic, intestinal, and neural stem cells in mice (20-22). Here, an increase in a population of cells expressing markers of kidney progenitor cells (CD24⁺CD133⁺CD45⁻) (59-61) was identified in the blood of patients after FMD cycles. Whereas the exact origin of these “circulating kidney progenitor-like cells” has not yet been established and warrants further investigation, it is believed that the increase in these cells detected after the FMD may be important because it was observed in association with the amelioration of the kidney and other physiological parameters. The results indicate a threefold increase in the number of circulating CD24⁺CD133⁺CD45⁻ cells after FMD intervention in the 13 patients compared with levels before undergoing dietary treatment [FIG. 7N; in healthy patients, these cells are present in 0.4%. The same population of circulating putative progenitor cells was also detected in rats exposed to one cycle of LS-FMD and 72 hours after refeeding. A population of resident CD24⁺CD133⁺CD45⁻ “progenitor-like” cells, mostly confined to the tubular compartments of the kidney, was also identified through the snRNA-seq and ST data.

It is also known that macrophages play an important role in reestablishing tissue homeostasis after injury in many different organs, including the kidney (62, 63), by regulating recruitment of different stem cells/progenitors in the site of injury, in addition to remodeling matrix composition and fibrosis. Using the snRNA-seq data, an immune component cluster represented by macrophages and monocytes was also identified. Subclustering identified four different clusters, and the EoD group, compared with the PAN group (specifically in C1), was characterized by differentially expressed genes and pathways that involve stem cell recruitment and differentiation, whereas C2 of the EoD group and the 24-hours-after-refeeding group (in C1, C2, and C3) were more closely represented by stem cell maintenance, in addition to response to stress, matrix reorganization, and mammalian target of rapamycin (mTOR) signaling. A macrophage cluster (Csfr1⁺CD74⁺Ptprc⁺spots) was also identified in ST analysis and was predominantly represented in the PAN sample (38.04%) compared with that in healthy control (12.9%) and PAN/LS-FMD-treated (11.46%) rats, as shown by UMAP and spot distribution in spatial maps. These results not only support the feasibility and applicability of FMD in clinical settings but highlight the potential for FMD cycles to treat kidney disease possibly by also stimulating regenerative processes.

### DISCUSSION

Dietary interventions have been extensively investigated in the context of kidney disease. Nutritional recommendations are in place for patients with CKD to manage their disease, slowing down its progression and reducing associated risks, but their effects are limited and have not been associated with regeneration or major improvements in kidney function (12-17). Here, it is demonstrated that multiple cycles of the low-salt fasting-mimicking diet (LS-FMD) followed by refeeding were sufficient to achieve sustained improvements in kidney function, as evidenced by reductions in albumin-to-creatinine ratio (ACR) and blood urea nitrogen (BUN) levels in puromycin aminonucleoside (PAN)-injured rats. Additionally, the beneficial effects of this dietary regimen were confirmed in a lupus nephritis-associated chronic kidney disease (CKD) mouse model, indicating that LS-FMD cycles are effective across different forms of CKD and enhancing their potential translatability to diverse progressive kidney diseases.

The improved physiological outcome correlated with preservation of kidney morphology (glomeruli and tubules) and with a reduction of glomerulosclerosis, a hallmark of most glomerulopathies (6, 7). FMD was applied after the acute phase of injury in the model. Therefore, FMD cycles should also be evaluated in clinical trials for their effect in preventing CKD development and progression after episodes of acute kidney injury.

LS-FMD induced transcriptional changes, which resulted in long-term improvements of the function and structure of the kidney compartments, in particular, of glomeruli. Therefore, LS-FMD contributes to the preservation of the podocyte pool and, thus, maintenance of glomerular homeostasis. It was identified that the low-salt fasting-mimicking diet (LS-FMD) preserved the functional podocyte pool through regulation of the podocyte cell cycle. In addition, LS-FMD regulated podocyte regeneration/reprogramming by activating a progenitor lineage and trans-differentiation within the glomerulus. Cell cycle progression is sensitive to nutrient availability, and conditions of starvation result in accumulation of cell cycle regulatory proteins (64-67). When podocytes are under stress, they initially become hypertrophic and eventually attempt to divide by entering the G₁ phase, leading to mitotic catastrophe and detachment (6, 53). One cycle of LS-FMD resulted in modulation of the cell cycle regulators, which promoted maintenance of the podocytes' quiescent state (Go phase of the cell cycle), thus preventing their ultimate loss, as evidenced in the FUCCI mouse model.

snRNA-seq confirmed the presence of multiple states in podocytes undergoing LS-FMD and refeeding, demonstrating rapid transcriptional changes that recapitulated part of glomerular development. At the end of the LS-FMD, PCR analyses, snRNA-seq, Western blotting, and histology all confirmed the expression of developmental, regenerative, and trans-differentiation markers including Myc, Nanog, Pax8, and Cd24, several of which continue to be expressed at 24 hours of refeeding. At 24 hours after refeeding, snRNA-seq also revealed the presence of autophagic processes and epithelial cell differentiation that culminated at 72 hours, with reinforcement of the podocyte program and the enrichment of differentiation pathways. This time-specific expression points to the activation of progenitor cells at the end of the diet, with their migration and trans-differentiation also corroborated by the presence of PAX8+/NEPHRIN⁺ cells within the glomerular tuft.

A possible contributing factor consistent with the data is that mature podocytes might undergo a reprogramming in response to LS-FMD and revert to a more progenitor-like cell able to contribute to the regenerative effects that were observed. The increase and activation of stem cells or reprogramming in the bone marrow, intestine, pancreas, and nervous system (20-24) in response to fasting/FMD refeeding demonstrated in mice appears to be related to organ or system atrophy during the fasting/FMD followed by reexpansion after FMD with refeeding (25). The atrophy may contribute to the breakdown of damaged macromolecules and organelles, in part through the activation of autophagy, whereas the reexpansion may use stem, progenitors, and cellular reprogramming to regenerate various systems through a coordinated developmental-like program.

LS-FMD promoted transcriptional changes not only in podocytes but also in GECs and mesangial cells as well as in cells of the proximal tubule and LOH. Clusters of cells exhibiting developmental features were identified across multiple kidney cell types, confirming the potential of the low-salt fasting-mimicking diet (LS-FMD) to stimulate endogenous regeneration and restore normal renal function. Although puromycin primarily induces damage in podocytes, extensive injury involving other kidney compartments was observed in PAN-treated rats. Administration of the fasting-mimicking diet (FMD) conferred protection and facilitated restoration of most renal structures. Notably, an upregulation of the transcription factor Zbtb16 was detected in the majority of nuclei, suggesting activation of regenerative transcriptional programs. Zbtb16 might function as an important nutrient sensing effector in response to fasting in different cell types because it was shown to coordinate the response to nutrient deficit in the hypothalamus (68). Zbtb16 has also been described as a downstream effector of the glucocorticoid pathway to enhance podocyte stability (69).

The study has limitations. It is acknowledged the limited number of samples used for the ST analysis. Because many of the previous studies evaluating the FMD in different animal models were performed in male mice, for the consistency and the reproducibility of the data, it was decided to use male rats in the studies described. However, for the study of the lupus nephritis model, female mice were used because they are more susceptible to damage than males. Many different mouse and rat CKD animal models are available, and it is acknowledged that a perfect model comparable to the human pathophysiology to perform dietary studies might not exist because all animal models of CKD present with some limitations (70, 71).

Considering the chronic nature of progressive CKD and the occurrence of multiple insults, studying the application of multiple rounds of FMD cycles between reinjury episodes and longer post-FMD monitoring will be needed to determine the long-term effects of FMD in reversing CKD. Limitations of the clinical trial include the small size and the lack of differences between groups at the end of the intervention.

In summary, the study indicates that the regenerative effects of FMD cycles observed in mice also occur in rats and are able to reverse the kidney damage caused by a potent nephrotoxin, thus suggesting that the effects of fasting-refeeding cycles on multisystem regeneration may be applied to many mammalian organisms. The preliminary results from a pilot human clinical study also indicate that the FMD is safe in terms of maintenance of the lean and muscle mass and is well tolerated in patients with stage 3 CKD. This was a major goal of the pilot study because the CKD population is at risk of malnutrition.

This preliminary clinical study provides initial evidence for the effect of FMD cycles in stabilizing kidney disease and, in some cases, inducing a moderate amelioration of renal function for at least 1 year. These encouraging preliminary results support the feasibility of FMD and the need for large randomized clinical studies to evaluate whether the regenerative and disease-reversing effects observed in rats may also be observed in humans.

### MATERIALS AND METHODS

### Study Design

This study was designed to investigate the role of the fasting-mimicking diet (FMD) in the context of chronic kidney disease (CKD). The model of puromycin aminonucleoside (PAN)-induced nephrosis in rats was first validated, and a kidney-specific fasting-mimicking diet, termed the low-salt FMD (LS-FMD), was developed. Following establishment of PAN-induced renal injury, rats were administered six cycles of LS-FMD or standard FMD, and both short- and long-term analyses were conducted to assess the structural and functional effects of the dietary interventions. Additionally, metabolic profiling, single-cell RNA sequencing (scRNA-seq), and microarray analyses were performed in PAN-injured rats after a single LS-FMD cycle to characterize the metabolic and transcriptional changes induced by the diet. The protective effects of LS-FMD were further evaluated using two distinct genetic mouse models of kidney disease. To assess the therapeutic potential of the FMD in humans, a pilot clinical study was conducted in patients with stage 3 chronic kidney disease. Patients were treated with the three cycles of FMD. Sample sizes were determined to ensure 95% probability of identifying effects by two-tailed unpaired or paired Student's t test or by either one-way analysis of variance (ANOVA) or a two-way ANOVA with either Tukey's, Bonferroni, or Fisher's test correction. Each time point represents a biological replicate, and all the sample numbers per experimental groups are reported in the legends. No data was excluded from the study, and all rats and mice were randomly allocated within the experimental groups. The investigators were not blinded for the allocation or the data analysis. For the pilot clinical study, 13 patients were enrolled, and patient characteristics are reported in Table 1. No patient was excluded from the study, and the investigators were not blinded during the analysis of patient data. The pilot clinical study was approved by the Institutional Review Board of the University La Sapienza, Roma. All the rat and mouse experiments were approved by the Institutional Animal Care and Use Committee (IACUC) at Children's Hospital Los Angeles (CHLA) and Icahn School of Medicine at Mount Sinai.

### Animals

All animal studies were performed under approved protocol of the IACUC at CHLA. Male Sprague-Dawley rats, 6 to 8 weeks old, were purchased from Charles River (strain code: 400), and a total of n=93 rats were used for all experiments. The FUCCI mice [B6; 129-Gt(ROSA)26Sor&lt; tml(Fucci2aR)Jkn&gt; stock no. RBRC06511 obtained from the Riken BRC Experimental Animal Division] were already available in our laboratory (56). FUCCI mice were crossed with X-linked Alport syndrome mice, which carry a Col4(5 mutation (B6.Cg-Col4α5^{tmlYseg}/J, stock no. 006283) (72), also already present in our laboratory. A total of n=10 AS-FUCCI mice were used for the experiment on cell cycle analysis.

MRL/MpJ-Faslpr/J (MRL/lpr) mice were bred at Icahn School of Medicine at Mount Sinai. All the experiments were performed in littermates maintained in the same room to limit potential influences of different microbiomes, but FMD and ad libitum MRL/lpr mice were housed in separate cages. All MRL/MpJ-Faslpr/J mice were housed in the Center for Comparative Medicine and Surgery at the Icahn School of Medicine at Mount Sinai under IACUC in accordance with guidelines of the Association for Assessment and Accreditation of Laboratory Animal Care International.

### PAN Induction of Nephrosis

Sprague-Dawley male rats, 12 to 16 weeks of age, were administered PAN (Sigma-Aldrich, no. 58-60-6) by intraperitoneal injection at a dose of 40 mg/kg of body weight. A fresh PAN solution was prepared each time in distilled water (40 mg/ml) and sterilized by filtration through a 0.2-μm syringe filter before injection, and an average of 0.2 ml of solution was injected per rat, based on body weight.

### Urine, Blood Collection, Proteinuria, and BUN Measurements

Urine and blood samples were routinely collected before PAN injection, 3 and 10 days after injection, and then on the last day of every cycle of diet. Samples were also collected at 10 days, 4 weeks, and 6 weeks after the end of treatment (see timeline in FIG. 2A). For urine collection, each rat was placed in a clean cage, and the urine was collected from the bottom of the cage, on the spot. Blood samples were collected with rats under isoflurane anesthesia by cutting the tail tip and milking the rat tail into BD Microtainer plasma separation tubes with lithium heparin (BD, no. 365987). Blood samples were centrifuged for 3 min at 13,000 rpm. All samples were stored at −80° C. until further use. Proteinuria was determined using an enzyme-linked immunosorbent assay for rat albuminuria (Immunology Consultants Laboratory, no. E90AL) and a quantitative colorimetric assay kit for creatinine (BioAssay Systems, no. DICT-500), following the manufacturer's protocols, respectively. The ACR, as a measure of renal function, was lastly calculated by normalizing the albumin output with the urine creatinine. BUN was measured by colorimetric assay (BioAssay Systems, no. DIUR-100) following the manufacturer's instructions. For mouse studies, urine creatinine and albumin were quantified using commercial kits from Cayman Chemical (no. 500701) and Bethyl Laboratory Inc. (no. E99-134), respectively. For BUN, a colorimetric detection kit was used (Thermo Fisher Scientific, no. EIABUN).

### Assessment of Tubular Injury

Systematic histological scoring of tubular injury was performed on kidney sections from healthy rats and PAN rats either fed ad libitum or with FMD and LS-FMD and evaluated at different time points along disease progression, based on the published protocols (73, 74). In brief, damage, such as tubular dilation, vacuolization, brush border loss, cellular cast, fibrosis, and infiltration; red blood cells; and hyaline casts were given a score between 0 and 4 as a percentage of the total affected cortical area in periodic acid-Schiff (PAS)-stained kidney sections (score: 0, <1%; 1, ≥1 to 10%; 2, ≥10 to 25%; 3, ≥25 to 50%; and 4, >50%), averaged from n=20 to 30 cortical fields (×10 magnification) across n=3 different kidney sections (n=10 areas per section) for each animal (n=3 to 5 per group). Scoring results were visualized in heatmaps.

### Glomerulosclerosis Index and Glomerular Count

For morphometric evaluation, the cross-sectional area of all consecutive glomerular profiles contained in five kidneys sections per animal, 50 m apart were considered. An average of 25 glomeruli per animal were randomly selected, and the degree of glomerular damage was assessed using a semiquantitative scoring method: grade 0, no observed change; grade 1, sclerotic area up to 25% of glomerulus (minimal sclerosis); grade 2, sclerotic area of 25 to 50% (moderate sclerosis); grade 3, sclerotic area>50% but not global (moderate-severe sclerosis); and grade 4, sclerotic area of 75 to 100% (severe sclerosis). The GSI was calculated using the following formula: GSI=(1×n₁)+(2×n₂)+(3×n₃)+(4×n₄)/n₀+n₁+n₂+n₃+n₄, where nₓ is the number of glomeruli in each grade of glomerulosclerosis (75, 76). For glomeruli count, n=3 or 4 samples were included per group. n=3 separate sections were cut from each sample at a 100-μm distance and stained with hematoxylin and eosin (H&E). For each section, glomeruli from 10 different areas (microscope quadrant) of the kidney were counted and averaged.

### FMD and LS-FMD

The rat version of the FMD is a 4-day regimen using ingredients like those used for the human FMD (25). Two versions of the FMD diet were tested on the PAN rat model: an FMD, previously published (23-25) [see formulation of the regular FMD in (23)], and a version with lower electrolyte concentrations (potassium, sodium, and phosphorus) referred to as LS-FMD. The source of calories was 42% from carbohydrates, 2% protein, and 47% fat. The diets were mainly composed of extra virgin olive oils and vegetable soups. All diet ingredients were thoroughly mixed and then blended together with heated hydrogel (Clear H₂O).

The diet, in rats, was started 14 days after PAN injection. Rats on the FMD diet were fed 50% of the standard daily calorie intake on day 1 (1.9 kcal/g) and 10% of normal daily calorie intake on days 2 to 4 (0.26 kcal/g). Rats on the LS-FMD were fed 16 g of diet for 4 days, providing 10% of daily calorie intake (on days 1 to 4) based on the results from the metabolic analysis that show that rats eat around 78 to 80 kcal/day of a regular chow diet. The kilocalorie content of the LS-FMD was 0.543 kcal/g. Before supplying the FMD diet, animals were transferred into fresh cages to avoid feeding on residual chow and coprophagy. All animals always had access to water. All rats were supplied with fresh food during the morning hours (8:00 a.m. to 10:00 a.m.). Rats on FMD and LS-FMD generally consumed the supplied food within the first few hours of the light cycle. Control animals fed ad libitum usually consumed the supplied food during the dark hours. Rats in the diet groups were monitored daily for weight loss. Animals were immediately refed with regular chow when ≥20% body weight was lost before the end of the cycle. After the end of each FMD or LS-FMD cycle, rats were fed ad libitum regular chow for up to 10 days to regain body weight before the next cycle. For the multiple cycle experiment, rats were fed the diet on alternate weeks to allow the rats to recover the original body weight between one cycle and the next. A total of six cycles were administered. For the FUCCI mice, the same diet was used and fed to them at 4.6 g of LS-FMD per day of diet. The daily intake was calculated to be 10% of the regular caloric intake on the basis of previously published metabolic data (23). FUCCI mice were subjected to one cycle of LS-FMD and refeeding.

For the MRL/MpJ-Faslpr/J (lupus nephritis) mouse model, mice were fed with weekly cycles of standard FMD starting at 1 month of age or ad libitum for 15 weeks. The weight of the animals was measured throughout the duration of the experiment.

### Clinical Study

The clinical trial to evaluate the effect of FMD in patients with CKD was registered at the ClinicalTrials.gov (NCT04490252). The present study is a controlled, prospective, longitudinal clinical pilot study started in April 2018 at various institutions affiliated with the Department of Translational and Precision Medicine of the University “Umberto I” Rome's hospital. Individuals were recruited on the basis of approval from the ethic committee (ref number 4799) based on established inclusion (volunteer patients, 18 to 65 years of age, affected by CKD stage G3 KDIGO, 30 ml/min<eGFR<60 ml/min, and diagnosis of primary glomerulonephritis) and exclusion [secondary glomerulonephritis, severe heart disease (stage G3 KDOQI) or acute heart failure, severe ongoing infection, congenital heart disease, ongoing neoplasia, chronic liver disease, chronic intestinal disease, chronic respiratory disease, cerebrovascular disease, acute myocardial infarction, valvular heart disease, acute coronary syndrome within 3 months from the start of the diet, refusing informed consent, pregnancy, breastfeeding, or plan for pregnancy] criteria. All participants signed informed consent forms and were not offered financial compensation for participation.

Prespecified outcome measures include adherence to the dietary protocol and evaluation of physiological markers before and after completion of the study. Examinations included body composition, serum level of IGF-1, markers of inflammation and oxidative stress, cardiovascular and endothelial risk factors, markers of renal function, regenerative markers (circulating stem cells), and psycho-cognitive evaluation. Parameters were measured before and after FMD intervention, and some parameters were also assessed during a regular follow-up at 1 year after the end of the intervention. Parameters were also measured before and after 3 months on a free diet.

### Human FMD

The human version of the FMD comprises proprietary formulations, belonging to University of Southern California and L-Nutra (www.prolonfmd.com), of vegetable-based soups, energy bars, energy drinks, chip snacks, tea, and a supplement providing high minerals, vitamins, and essential fatty acids, as previously published (77). The FMD is a plant-based diet designed to attain fasting-like effects on the serum IGF-1, IGFBP-1, glucose, and ketone bodies while providing both macro- and micronutrients to minimize the burden of fasting and adverse effects (25). The FMD diet consisted of a 5-day-per-month regimen: day 1, 1090 kcal (10% protein, 56% fat, and 34% carbohydrate). Days 2 to 5 were identical in formulation with 725 kcal (9% protein, 44% fat, and 47% carbohydrate). All items to be consumed per day were individually boxed to allow the participants to choose when to eat and to avoid accidentally consuming components of the following day.

### Statistical Analysis

Statistical analysis was performed using the Prism software (version 9.2.0, GraphPad). All groups and experiments analyzed, except for the ST, have n=3 or more samples; details are in the figure legends. All results are presented as either median or means±SD or SEM. All comparisons between two groups were analyzed with a two-tailed unpaired or paired Student's t test for populations with equal variance. Comparisons of more than two groups and repeated measures were performed using either one-way ANOVA or a two-way ANOVA with mixed-effects analysis, multiple comparison analysis, or two-way analysis of variance with either Tukey's; Bonferroni; Dunnett's; Benjamini, Krieger, and Yekutieli; Sidik's; or Fisher's least significance test correction for multiple comparisons, as applicable. Significance is expressed with P values: *P<0.05, **P<0.01, ***P<0.001, and ****P<0.0001.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

1. GBD Chronic Kidney Disease Collaboration, Global, regional, and national burden of chronic kidney disease, 1990-2017: A systematic analysis for the Global Burden of Disease Study 2017. Lancet 395, 709-733 (2020).
2. K. M. Kim, H. J. Oh, H. Y. Choi, H. Lee, D.-R. Ryu, Impact of chronic kidney disease on mortality: A nationwide cohort study. Kidney Res. Clin. Pract. 38, 382-390 (2019).
3. C. P. Kovesdy, Epidemiology of chronic kidney disease: An update 2022. Kidney Int. Suppl. 12, 7-11 (2022).
4. E. J. Weil, K. V. Lemley, C. C. Mason, B. Yee, L. I. Jones, K. Blouch, T. Lovato, M. Richardson, B. D. Myers, R. G. Nelson, Podocyte detachment and reduced glomerular capillary endothelial fenestration promote kidney disease in type 2 diabetic nephropathy. Kidney Int. 82, 1010-1017 (2012).
5. W. Kriz, K. V. Lemley, A potential role for mechanical forces in the detachment of podocytes and the progression of CKD. J. Am. Soc. Nephrol. 26, 258-269 (2015).
6. K. V. Lemley, Glomerular pathology and the progression of chronic kidney disease. Am. J. Physiol. Renal Physiol. 310, F1385-F1388 (2016).
7. M. Nagata, Podocyte injury and its consequences. Kidney Int. 89, 1221-1230 (2016).
8. E. Wuhl, F. Schaefer, Therapeutic strategies to slow chronic kidney disease progression. Pediatr. Nephrol. Berl. Ger. 23, 705-716 (2008).
9. X. Xie, Y. Liu, V. Perkovic, X. Li, T. Ninomiya, W. Hou, N. Zhao, L. Liu, J. Lv, H. Zhang, H. Wang, Renin-angiotensin system inhibitors and kidney and cardiovascular outcomes in patients with CKD: A Bayesian network meta-analysis of randomized clinical trials. Am. J. Kidney Dis. 67, 728-741 (2016).
10. H. J. L. Heerspink, B. V. Stefinsson, R. Correa-Rotter, G. M. Chertow, T. Greene, F.-F. Hou, J. F. E. Mann, J. J. V. McMurray, M. Lindberg, P. Rossing, C. D. Sjostrom, R. D. Toto, A.-M. Langkilde, D. C. Wheeler, Dapagliflozin in patients with chronic kidney disease. N. Engl. J. Med. 383, 1436-1446 (2020).
11. J. J. Garcia Sanchez, J. Thompson, D. A. Scott, R. Evans, N. Rao, E. Sorstadius, G. James, S. Nolan, E. T. Wittbrodt, A. Abdul Sultan, B. V. Stefansson, D. Jackson, K. R. Abrams, Treatments for chronic kidney disease: A systematic literature review of randomized controlled trials. Adv. Ther. 39, 193-220 (2022).
12. S. Klahr, A. S. Levey, G. J. Beck, A. W. Caggiula, L. Hunsicker, J. W. Kusek, G. Striker, The effects of dietary protein restriction and blood-pressure control on the progression of chronic renal disease. N. Engl. J. Med. 330, 877-884 (1994).
13. C. A. M. Anderson, H. A. Nguyen, D. E. Rifkin, Nutrition interventions in chronic kidney disease. Med. Clin. North Am. 100, 1265-1283 (2016).
14. L. Garneata, A. Stancu, D. Dragomir, G. Stefan, G. Mircescu, Ketoanalogue-supplemented vegetarian very low-protein diet and CKD progression. J. Am. Soc. Nephrol. 27, 2164-2176 (2016).
15. K. Kalantar-Zadeh, S. Joshi, R. Schlueter, J. Cooke, A. Brown-Tortorici, M. Donnelly, S. Schulman, W.-L. Lau, C. M. Rhee, E. Streja, E. Tantisattamo, A. J. Ferrey, R. Hanna, J. L. T. Chen, S. Malik, D. V. Nguyen, S. T. Crowley, C. P. Kovesdy, Plant-dominant low-protein diet for conservative management of chronic kidney disease. Nutrients 12, 1931 (2020).
16. P. Molina, E. Gavela, B. Vizcaino, E. Huarte, J. J. Carrero, Optimizing diet to slow CKD progression. Front. Med. 8, 654250 (2021).
17. H. L. MacLaughlin, A. N. Friedman, T. A. Ikizler, Nutrition in kidney disease: Core curriculum 2022. Am. J. Kidney Dis. 79, 437-449 (2022).
18. J. A. Torres, S. L. Kruger, C. Broderick, T. Amarlkhagva, S. Agrawal, J. R. Dodam, M. Mrug, L. A. Lyons, T. Weimbs, Ketosis ameliorates renal cyst growth in polycystic kidney disease. Cell Metab. 30, 1007-1023.e5 (2019).
19. S. Strubl, S. Oehm, J. A. Torres, F. Grundmann, J. Haratani, M. Decker, S. Vuong, A. Kaur Bhandal, N. Methot, R. Haynie-Cion, F. Meyer, F. Siedek, U. Korst, R.-U. Müller, T. Weimbs, Ketogenic dietary interventions in autosomal dominant polycystic kidney disease—a retrospective case series study: First insights into feasibility, safety and effects. Clin. Kidney J. 15, 1079-1092 (2022).
20. C.-W. Cheng, G. B. Adams, L. Perin, M. Wei, X. Zhou, B. S. Lam, S. Da Sacco, M. Mirisola, D. I. Quinn, T. B. Dorff, J. J. Kopchick, V. D. Longo, Prolonged fasting reduces IGF-1/PKA to promote hematopoietic-stem-cell-based regeneration and reverse immunosuppression. Cell Stem Cell 14, 810-823 (2014).
21. P. Rangan, I. Choi, M. Wei, G. Navarrete, E. Guen, S. Brandhorst, N. Enyati, G. Pasia, D. Maesincee, V. Ocon, M. Abdulridha, V. D. Longo, Fasting-mimicking diet modulates microbiota and promotes intestinal regeneration to reduce inflammatory bowel disease pathology. Cell Rep. 26, 2704-2719.e6 (2019).
22. P. Rangan, F. Lobo, E. Parrella, N. Rochette, M. Morselli, T.-L. Stephen, A. L. Cremonini, L. Tagliafico, A. Persia, I. Caffa, F. Monacelli, P. Odetti, T. Bonfiglio, A. Nencioni, M. Pigliautile, V. Boccardi, P. Mecocci, C. J. Pike, P. Cohen, M. J. LaDu, M. Pellegrini, K. Xia, K. Tran, B. Ann, D. Chowdhury, V. D. Longo, Fasting-mimicking diet cycles reduce neuroinflammation to attenuate cognitive decline in Alzheimer's models. Cell Rep. 40, 111417 (2022).
23. C.-W. Cheng, V. Villani, R. Buono, M. Wei, S. Kumar, O. H. Yilmaz, P. Cohen, J. B. Sneddon, L. Perin, V. D. Longo, Fasting-mimicking diet promotes Ngn3-driven 3-cell regeneration to reverse diabetes. Cell 168, 775-788.e12 (2017).
24. I. Y. Choi, L. Piccio, P. Childress, B. Bollman, A. Ghosh, S. Brandhorst, J. Suarez, A. Michalsen, A. H. Cross, T. E. Morgan, M. Wei, F. Paul, M. Bock, V. D. Longo, A diet mimicking fasting promotes regeneration and reduces autoimmunity and multiple sclerosis symptoms. Cell Rep. 15, 2136-2146 (2016).
25. S. Brandhorst, I. Y. Choi, M. Wei, C. W. Cheng, S. Sedrakyan, G. Navarrete, L. Dubeau, L. P. Yap, R. Park, M. Vinciguerra, S. Di Biase, H. Mirzaei, M. G. Mirisola, P. Childress, L. Ji, S. Groshen, F. Penna, P. Odetti, L. Perin, P. S. Conti, Y. Ikeno, B. K. Kennedy, P. Cohen, T. E. Morgan, T. B. Dorff, V. D. Longo, A periodic diet that mimics fasting promotes multi-system regeneration, enhanced cognitive performance, and healthspan. Cell Metab. 22, 86-99 (2015).
26. A. Sulaj, S. Kopf, E. von Rauchhaupt, E. Kliemank, M. Brune, Z. Kender, H. Bartl, F. G. Cortizo, K. Klepac, Z. Han, V. Kumar, V. Longo, A. Teleman, J. G. Okun, J. Morgenstern, T. Fleming, J. Szendroedi, S. Herzig, P. P. Nawroth, Six-month periodic fasting in patients with type 2 diabetes and diabetic nephropathy: A proof-of-concept study. J. Clin. Endocrinol. Metab. 107, 2167-2181 (2022).
27. S. M. Kim, J. Y. Jung, Nutritional management in patients with chronic kidney disease. Korean J. Intern. Med. 35, 1279-1290 (2020).
28. S. Yamada, M. Inaba, Potassium metabolism and management in patients with CKD. Nutrients 13, 1751 (2021).
29. C. D'Alessandro, G. B. Piccoli, A. Cupisti, The “phosphorus pyramid”: A visual tool for dietary phosphate management in dialysis and CKD patients. BMC Nephrol. 16, 9 (2015).
30. M. G. Vervloet, S. Sezer, Z. A. Massy, L. Johansson, M. Cozzolino, D. Fouque, The role of phosphate in kidney disease. Nat. Rev. Nephrol. 13, 27-38 (2017).
31. A. B. Nair, S. Jacob, A simple practice guide for dose conversion between animals and human. J. Basic Clin. Pharm. 7, 27-31 (2016).
32. F. Salmen, P. L. Stihl, A. Mollbrink, J. F. Navarro, S. Vickovic, J. Frisen, J. Lundeberg, Barcoded solid-phase RNA capture for spatial transcriptomics profiling in mammalian tissue sections. Nat. Protoc. 13, 2501-2534 (2018).
33. L. Garcia-Alonso, V. Lorenzi, C. I. Mazzeo, J. P. Alves-Lopes, K. Roberts, C. Sancho-Serra, J. Engelbert, M. Mareckova, W. H. Gruhn, R. A. Botting, T. Li, B. Crespo, S. van Dongen, V. Y. Kiselev, E. Prigmore, M. Herbert, A. Moffett, A. Chedotal, O. A. Bayraktar, A. Surani, M. Haniffa, R. Vento-Tormo, Single-cell roadmap of human gonadal development. Nature 607, 540-547 (2022).
34. A. Petrosyan, V. Villani, P. Aguiari, M. E. Thornton, Y. Wang, A. Rajewski, S. Zhou, P. Cravedi, B. H. Grubbs, R. E. De Filippo, S. Sedrakyan, K. V. Lemley, M. Csete, S. Da Sacco, L. Perin, Identification and characterization of the wilms tumor cancer stem cell. Adv. Sci. 10, 2206787 (2023).
35. J. W. Lee, C.-L. Chou, M. A. Knepper, Deep sequencing in microdissected renal tubules identifies nephron segment-specific transcriptomes. J. Am. Soc. Nephrol. 26, 2669-2677 (2015).
36. J. Z. Clark, L. Chen, C.-L. Chou, H. J. Jung, J. W. Lee, M. A. Knepper, Representation and relative abundance of cell-type selective markers in whole-kidney RNA-Seq data. Kidney Int. 95, 787-796 (2019).
37. P. T. Brinkkoetter, T. Bork, S. Salou, W. Liang, A. Mizi, C. Ozel, S. Koehler, H. H. Hagmann, C. Ising, A. Kuczkowski, S. Schnyder, A. Abed, B. Schermer, T. Benzing, O. Kretz, V. G. Puelles, S. Lagies, M. Schlimpert, B. Kammerer, C. Handschin, C. Schell, T. B. Huber, Anaerobic glycolysis maintains the glomerular filtration barrier independent of mitochondrial metabolism and dynamics. Cell Rep. 27, 1551-1566.e5 (2019).
38. J. W. Kim, H. W. Kim, S. A. Nam, J. Y. Lee, H. J. Cho, T.-M. Kim, Y. K. Kim, Human kidney organoids reveal the role of glutathione in Fabry disease. Exp. Mol. Med. 53, 1580-1591 (2021).
39. H. Ma, D. G. Sandor, L. H. Beck, The role of complement in membranous nephropathy. Semin. Nephrol. 33, 531-542 (2013).
40. J. Huang, Z. Cui, Q. Gu, Y. Zhang, Z. Qu, X. Wang, F. Wang, X. Cheng, L. Meng, G. Liu, M. Zhao, Complement activation profile of patients with primary focal segmental glomerulosclerosis. PLOS ONE 15, e0234934 (2020).
41. K. Takahashi, S. Yamanaka, Induction of pluripotent stem cells from mouse embryonic and adult fibroblast cultures by defined factors. Cell 126, 663-676 (2006).
42. S. J. Shankland, J. W. Pippin, J. S. Duffield, Progenitor cells and podocyte regeneration. Semin. Nephrol. 34, 418-428 (2014).
43. K. Berger, K. Schulte, P. Boor, C. Kuppe, T. H. van Kuppevelt, J. Floege, B. Smeets, M. J. Moeller, The regenerative potential of parietal epithelial cells in adult mice. J. Am. Soc. Nephrol. 25, 693-705 (2014).
44. D. G. Eng, M. W. Sunseri, N. V. Kaverina, S. S. Roeder, J. W. Pippin, S. J. Shankland, Glomerular parietal epithelial cells contribute to adult podocyte regeneration in experimental focal segmental glomerulosclerosis. Kidney Int. 88, 999-1012 (2015).
45. L. Ni, C. Yuan, X. Wu, The recruitment mechanisms and potential therapeutic targets of podocytes from parietal epithelial cells. J. Transl. Med. 19, 441 (2021).
46. F. Ding, X. Tian, J. Mo, B. Wang, J. Zheng, Determination of the dynamic cellular transcriptional profiles during kidney development from birth to maturity in rats by single-cell RNA sequencing. Cell Death Discov. 7, 162 (2021).
47. A. Schumacher, M. B. Rookmaaker, J. A. Joles, R. Kramann, T. Q. Nguyen, M. van Griensven, V. L. S. LaPointe, Defining the variety of cell types in developing and adult human kidneys by single-cell RNA sequencing. NPJ Regen. Med. 6, 45 (2021).
48. Z. Li, X. Guo, X. Quan, C. Yang, Z. Liu, H. Su, N. An, H. Liu, The role of parietal epithelial cells in the pathogenesis of podocytopathy. Front. Physiol. 13, 832772 (2022).
49. S. J. Shankland, H.-J. Anders, P. Romagnani, Glomerular parietal epithelial cells in kidney physiology, pathology, and repair. Curr. Opin. Nephrol. Hypertens. 22, 302-309 (2013).
50. C. B. Marshall, S. J. Shankland, Cell cycle regulatory proteins in podocyte health and disease. Nephron Exp. Nephrol. 106, e51-e59 (2007).
51. S. U. Vogelmann, W. J. Nelson, B. D. Myers, K. V. Lemley, Urinary excretion of viable podocytes in health and renal disease. Am. J. Physiol. Renal Physiol. 285, F40-F48 (2003).
52. W. Kriz, I. Shirato, M. Nagata, M. LeHir, K. V. Lemley, The podocyte's response to stress: The enigma of foot process effacement. Am. J. Physiol. Renal Physiol. 304, F333-F347 (2013).
53. S. R. Mulay, D. Thomasova, M. Ryu, O. P. Kulkarni, A. Migliorini, H. Bruns, R. Gröbmayr, E. Lazzeri, L. Lasagni, H. Liapis, P. Romagnani, H.-J. Anders, Podocyte loss involves MDM2-driven mitotic catastrophe. J. Pathol. 230, 322-335 (2013).
54. M. Hagen, E. Pfister, A. Kosel, S. Shankland, J. Pippin, K. Amann, C. Daniel, Cell cycle re-entry sensitizes podocytes to injury induced death. Cell Cycle 15, 1929-1937 (2016).
55. J. M. Basgen, J. S. Wong, J. Ray, S. B. Nicholas, K. N. Campbell, Podocyte foot process effacement precedes albuminuria and glomerular hypertrophy in CD2-associated protein deficient mice. Front. Med. 8, 745319 (2021).
56. C. N. Frank, X. Hou, A. Petrosyan, V. Villani, R. Zhao, J. R. Hansen, G. Clair, F. Salem, R. E. De Filippo, P. Cravedi, K. V. Lemley, L. Perin, Effect of disease progression on the podocyte cell cycle in Alport syndrome. Kidney Int. 101, 106-118 (2022).
57. U. K. Seidel, J. Gronewold, M. Volsek, O. Todica, A. Kribben, H. Bruck, D. M. Hermann, Physical, cognitive and emotional factors contributing to quality of life, functional health and participation in community dwelling in chronic kidney disease. PLOS ONE 9, e91176 (2014).
58. Kidney Disease: Improving Global Outcomes (KDIGO) Blood Pressure Work Group, KDIGO 2021 clinical practice guideline for the management of blood pressure in chronic kidney disease. Kidney Int. 99, S1-S87 (2021).
59. S. Gupta, C. Verfaillie, D. Chmielewski, S. Kren, K. Eidman, J. Connaire, Y. Heremans, T. Lund, M. Blackstad, Y. Jiang, A. Luttun, M. E. Rosenberg, Isolation and characterization of kidney-derived stem cells. J. Am. Soc. Nephrol. 17, 3028-3040 (2006).
60. P. Romagnani, G. Remuzzi, CD133+ renal stem cells always co-express CD24 in adult human kidney tissue. Stem Cell Res. 12, 828-829 (2014).
61. J. Huang, Y. Kong, C. Xie, L. Zhou, Stem/progenitor cell in kidney: Characteristics, homing, coordination, and maintenance. Stem Cell Res. Ther. 12, 197 (2021).
62. R. M. B. Bell, B. R. Conway, “One, no one, one hundred thousand—the multifaceted role of macrophages in health and disease—Part A” in International Review of Cell and Molecular Biology, S. A. Mariani, L. Cassetta, L. Galluzzi, Eds. (Academic Press, 2022), vol. 367, pp. 101-147.
63. E. Manole, C. Niculite, I. M. Lambrescu, G. Gaina, O. Ioghen, L. C. Ceafalan, M. E. Hinescu, Macrophages and stem cells-Two to tango for tissue repair?Biomolecules 11, 697 (2021).
64. B. L. Bohnsack, K. K. Hirschi, Nutrient regulation of cell cycle progression. Annu. Rev. Nutr. 24, 433-453 (2004).
65. C. R. McKeown, H. T. Cline, Nutrient restriction causes reversible G2 arrest in Xenopus neural progenitors. Development 146, dev178871 (2019).
66. G. Charbon, B. Mendoza-Chamizo, C. Campion, X. Li, P. R. Jensen, J. Frimodt-Møller, A. Lobner-Olesen, Energy starvation induces a cell cycle arrest in Escherichia coli by triggering degradation of the DnaA initiator protein. Front. Mol. Biosci. 8, 10.3389/fmolb.2021.629953 (2021).
67. D. Irvali, F. P. Schlottmann, P. Muralidhara, I. Nadelson, K. Kleemann, N. E. Wood, A. Doncic, J. C. Ewald, When yeast cells change their mind: Cell cycle “Start” is reversible under starvation. EMBO J. 42, el 10321 (2023).
68. H. Cheng, S. J. Pablico, J. Lee, J. S. Chang, S. Yu, Zinc finger transcription factor Zbtbl6 coordinates the response to energy deficit in the mouse hypothalamus. Front. Neurosci. 14, 592947 (2020).
69. H. Wang, A. Duan, J. Zhang, Q. Wang, Y. Xing, Z. Qin, Z. Liu, J. Yang, Glucocorticoid receptor wields chromatin interactions to tune transcription for cytoskeleton stabilization in podocytes. Commun. Biol. 4, 675 (2021).
70. J. Liang, Y. Liu, Animal models of kidney disease: Challenges and perspectives. Kidney 4, 1479-1493 (2023).
71. Y. W. Bao, Y. Yuan, J. H. Chen, W. Q. Lin, Kidney disease models: Tools to identify mechanisms and potential therapeutic targets. Zool. Res. 39, 72-86 (2018).
72. S. Sedrakyan, S. Da Sacco, A. Milanesi, L. Shiri, A. Petrosyan, R. Varimezova, D. Warburton, K. V. Lemley, R. E. De Filippo, L. Perin, Injection of amniotic fluid stem cells delays progression of renal fibrosis. J. Am. Soc. Nephrol. 23, 661-673 (2012).
73. S. Hakroush, D. Tampe, P. Korsten, P. Strobel, B. Tampe, Systematic scoring of tubular injury patterns reveals interplay between distinct tubular and glomerular lesions in ANCA-associated glomerulonephritis. J. Clin. Med. 10, 2682 (2021).
74. T. T. Pieters, L. L. Falke, T. Q. Nguyen, M. C. Verhaar, S. Florquin, F. J. Bemelman, J. Kers, T. Vanhove, D. Kuypers, R. Goldschmeding, M. B. Rookmaaker, Histological characteristics of acute tubular injury during delayed graft function predict renal function after renal transplantation. Physiol. Rep. 7, e14000 (2019).
75. T. Saito, E. Sumithran, E. F. Glasgow, R. C. Atkins, The enhancement of aminonucleoside nephrosis by the co-administration of protamine. Kidney Int. 32, 691-699 (1987).
76. C. Maric, K. Sandberg, C. Hinojosa-Laborde, Glomerulosclerosis and tubulointerstitial fibrosis are attenuated with 170-estradiol in the aging Dahl salt sensitive rat. J. Am. Soc. Nephrol. 15, 1546-1556 (2004).
77. M. Wei, S. Brandhorst, M. Shelehchi, H. Mirzaei, C. W. Cheng, J. Budniak, S. Groshen, W. J. Mack, E. Guen, S. Di Biase, P. Cohen, T. E. Morgan, T. Dorff, K. Hong, A. Michalsen, A. Laviano, V. D. Longo, Fasting-mimicking diet and markers/risk factors for aging, diabetes, cancer, and cardiovascular disease. Sci. Transl. Med. 9, eaai8700 (2017).
78. F. Furukawa, H. Tanaka, K. Sekita, T. Nakamura, Y. Horiguchi, Y. Hamashima, Dermatopathological studies on skin lesions of MRL mice. Arch. Dermatol. Res. 276, 186-194 (1984).
79. L.-J. Ma, A. B. Fogo, Model of robust induction of glomerulosclerosis in mice: Importance of genetic background. Kidney Int. 64, 350-355 (2003).
80, Y. Hao, S. Hao, E. Andersen-Nissen, W. M. Mauck, S. Zheng, A. Butler, M. J. Lee, A. J. Wilk, C. Darby, M. Zager, P. Hoffman, M. Stoeckius, E. Papalexi, E. P. Mimitou, J. Jain, A. Srivastava, T. Stuart, L. M. Fleming, B. Yeung, A. J. Rogers, J. M. McElrath, C. A. Blish, R. Gottardo, P. Smibert, R. Satija, Integrated analysis of multimodal single-cell data. Cell 184, 3573-3587.e29 (2021).
81. A. Dobin, C. A. Davis, F. Schlesinger, J. Drenkow, C. Zaleski, S. Jha, P. Batut, M. Chaisson, T. R. Gingeras, STAR: Ultrafast universal RNA-seq aligner. Bioinformatics 29, 15-21(2013).
82. L. Lins, F. M. Carvalho, SF-36 total score as a single measure of health-related quality of life: Scoping review. SAGE Open Med. 4, 2050312116671725 (2016).
83. P. Julayanont, S. Tangwongchai, S. Hemrungrojn, C. Tunvirachaisakul, K. Phanthumchinda, J. Hongsawat, P. Suwichanarakul, S. Thanasirorat, Z. S. Nasreddine, The Montreal Cognitive Assessment-Basic: A screening tool for mild cognitive impairment in illiterate and low-educated elderly adults. J. Am. Geriatr. Soc. 63, 2550-2554 (2015).
84. R. A. Steer, D. J. Rissmiller, A. T. Beck, Use of the Beck Depression Inventory-II with depressed geriatric inpatients. Behav. Res. Ther. 38, 311-318 (2000).
85. K. Kvaal, I. Ulstein, I. H. Nordhus, K. Engedal, The Spielberger State-Trait Anxiety Inventory (STAI): The state scale in detecting mental disorders in geriatric patients. Int. J. Geriatr. Psychiatry 20, 629-634 (2005).

What is claimed is:

1. A method for treating a subject having kidney disease comprising:
administering cycles of a low-salt fasting-mimicking diet to the subject having kidney disease, wherein each cycle comprises at least five days of restricted caloric intake, followed by a refeeding period, wherein the low-salt fasting-mimicking diet provides less than 2000 mg of sodium per day, less than 1500 mg of potassium per day, and less than 1500 mg of phosphorus per day, wherein the low-salt fasting-mimicking diet is designed as a plant-based regimen to provide macro- and micronutrients, including essential minerals, vitamins, and fatty acids, to attain fasting-like effects on serum IGF-1, IGFBP-1, glucose, and ketone bodies, while minimizing adverse effects associated with fasting.

2. The method of claim 1, wherein the low-salt fasting-mimicking diet provides less than about 1500 mg of sodium per day, 1000 mg of potassium per day, and less than 1000 mg of phosphorus per day.

3. The method of claim 1, wherein the low-salt fasting-mimicking diet provides less than about 1000 mg of sodium per day, 500 mg of potassium per day, and less than 500 mg of phosphorus per day.

4. The method of claim 1, wherein the low-salt fasting-mimicking diet modulates kidney cell regeneration, promotes a quiescent state in podocytes, and reduces glomerular sclerosis.

5. The method of claim 1, wherein the low-salt fasting-mimicking diet is administered for three to six cycles or more, each separated by an associated refeeding period of at least seven days.

6. The method of claim 1, wherein the low-salt fasting-mimicking diet provides a daily caloric intake for day one from 900 to 1300 kcal, with 5-15% of calories from protein, 45-65% of calories from fat, and 25-45% of calories from carbohydrate, and a daily caloric intake for days two to five from 600 to 800 kcal per day, with 5-15% of calories from protein, 35-55% of calories from fat, and 40-55% of calories from carbohydrates.

7. The method of claim 1, wherein the low-salt fasting-mimicking diet provides a daily caloric intake for day one from 1000 to 1200 kcal, with 8-12% of calories from protein, 50-60% of calories from fat, and 30-40% of calories from carbohydrates, and a daily caloric intake for days two to five from 700 to 750 kcal per day, with 7-11% of calories from protein, 40-50% of calories from fat, and 45-50% of calories from carbohydrates.

8. The method of claim 1, wherein the low-salt fasting-mimicking diet includes a range of individually boxed food items comprising vegetable-based soups, energy bars, energy drinks, chip snacks, and tea, along with a dietary supplement formulated to provide high minerals, vitamins, and essential fatty acids, allowing subjects to choose consumption times while avoiding unintended intake of components designated for different days.

9. The method of claim 1, wherein the low-salt fasting-mimicking diet induces metabolic changes in kidney cells, promoting gene expression associated with lipid metabolism, biological oxidation, and gluconeogenesis.

10. The method of claim 1, wherein the low-salt fasting-mimicking diet modulates expression of nephrogenic gene programs to reprogram renal cell types, including podocytes, glomerular endothelial cells, and proximal tubular cells.

11. The method of claim 1, wherein the low-salt fasting-mimicking diet modulates the cell cycle by inducing increase in modulators that inhibit cell cycle progression thereby promoting maintenance of podocytes in their quiescent and highly differentiated state.

12. The method of claim 1, wherein the kidney disease is chronic kidney disease (CKD).

13. The method of claim 12, wherein the low-salt fasting-mimicking diet provides sustained improvements in CKD for up to one year post-treatment.

14. The method of claim 12, wherein the low-salt fasting-mimicking diet reduces markers of inflammation and improves endothelial function in CKD patients.

15. The method of claim 1, wherein the low-salt fasting-mimicking diet further promotes an increase in circulating kidney progenitor cells characterized by CD24⁺CD133⁺CD45⁻ markers.

16. A method of treating a kidney disease in a human subject in need thereof, the method comprising administering to the subject a plurality of cycles of a fasting-mimicking diet (FMD) that is not a low-sodium fasting-mimicking diet (LS-FMD), each cycle comprising at least five consecutive days of reduced caloric intake followed by a refeeding period of at least seven days, wherein the reduced caloric intake provides:
(a) on day 900 to 1300 kilocalories with 5-15% of calories from protein, 40-65% of calories from fat, and 25-50% of calories from carbohydrate; and
(b) on each of days 2-5, 600 to 800 kilocalories with 5-15% of calories from protein, 35-55% of calories from fat, and 40-55% of calories from carbohydrates.

17. The method of claim 16, wherein the plurality of cycles consists of three cycles administered over about three months, each cycle being separated by 5-26 days of refeeding.

18. The method of claim 16, wherein the kidney disease is chronic kidney disease selected from the group consisting of IgA nephropathy, membranous nephropathy, focal segmental glomerulosclerosis, minimal change disease, membranoproliferative glomerulonephritis, lupus nephritis, diabetic nephropathy, and ischemic nephropathy.

19. The method of claim 16, wherein administering the FMD reduces proteinuria measured as 24-hour urinary protein or albumin-to-creatinine ratio and increases estimated glomerular filtration rate (eGFR) relative to baseline.

20. The method of claim 16, wherein administering the FMD increases circulating kidney progenitor-like cells characterized as CD24⁺CD133⁺CD45⁻ and improves endothelial function measured by brachial artery flow-mediated dilation.

21. A low-sodium fasting-mimicking diet composition for use in treating or preventing a kidney disease in a human subject, the low-sodium fasting-mimicking diet composition comprising:
daily meal portions configured for a fasting-mimicking cycle of at least 5 days, wherein, when the daily meal portions for a given day are consumed, the low-sodium fasting-mimicking diet composition provides less than about 2000 mg sodium per day, less than about 1500 mg potassium per day, and less than about 1500 mg phosphorus per day, and the daily meal portions providing for day 1, about 900 to 1300 kcal with about 5 to 15% of calories from protein, 45 to 65% fat, and 25 to 45% of calories from carbohydrate, and for each of days 2 to 5, about 600 to 800 kcal per day with 5-15% of calories from protein, 35-55% of calories from fat, and 40-55% of calories from carbohydrates.

22. The low-sodium fasting-mimicking diet composition of claim 21, wherein the composition provides no more than about 1500 mg sodium per day, no more than about 1000 mg potassium per day, and no more than about 1000 mg phosphorus per day.

23. The low-sodium fasting-mimicking diet composition of claim 21, wherein the composition provides no more than about 1000 mg sodium per day, no more than about 500 mg potassium per day, and no more than about 500 mg phosphorus per day.

24. The low-sodium fasting-mimicking diet composition of claim 21, wherein the sodium provided per day is between about 200 mg and about 1000 mg.

25. The low-sodium fasting-mimicking diet composition of claim 21, wherein across the fasting-mimicking cycle comprises about 38 to 45% carbohydrate, about 8 to 12% protein, and about 45-55% fat.

26. The low-sodium fasting-mimicking diet composition of claim 21, wherein the composition is ketogenic.

27. The low-sodium fasting-mimicking diet composition of claim 21, packaged as a pre-portioned diet package comprising five separately packaged daily portions configured for sequential consumption during the fasting-mimicking cycle.

28. The low-sodium fasting-mimicking diet composition of claim 27, wherein the pre-portioned diet package comprises one or more of:
a nut-based nutrition bar, a cocoa-based nutrition bar, a first olive-containing composition, a kale-cracker composition,
at least one vegetable soup;
a first vegetable broth;
a tea composition;
an energy-drink composition;
a micronutrient composition; and
an algal-oil composition, wherein the low-sodium fasting-mimicking diet composition optionally further comprises a mushroom soup, tomato soup, quinoa-minestrone, bean-minestrone, pumpkin soup, a second olive-containing composition, a second vegetable broth, lemon-spearmint tea, and hibiscus tea.

29. The low-sodium fasting-mimicking diet composition of claim 28, wherein each soup, broth, tea, and energy-drink composition is provided as a mix or concentrate to be reconstituted with added water at time of consumption.

30. The low-sodium fasting-mimicking diet composition of claim 28, wherein the energy-drink composition comprises glycerin and purified water as principal components, with glycerin present at about 20 to 60 wt % and purified water present at about 40 to 80 wt %.

31. The low-sodium fasting-mimicking diet composition of claim 28, wherein the algal-oil composition comprises Schizochytrium algal oil rich in docosahexaenoic acid (DHA), and the pre-portioned diet package provides an aggregate of about 600 mg DHA over the fasting-mimicking cycle.

32. The low-sodium fasting-mimicking diet composition of claim 28, wherein the micronutrient composition comprises vitamins A, C, D3, E, K, B1, B2, B3, B5, B6, B7, B9, and B12 and minerals Ca, Fe, Mg, Cu, Mn, Se, Cr, Mo, Zn, and I.

33. The low-sodium fasting-mimicking diet composition of claim 27, wherein each component is gluten-free or exceptionally low in gluten, such that each component contains less than 20 ppm gluten or 20 to 100 ppm gluten, respectively; and wherein serving-size ranges comprise: nut bar 30 to 60 g; cocoa bar 15 to 40 g; each olive composition 10 to 20 g; kale-cracker 20 to 60 g; each soup 20 to 50 g; first broth 5 to 15 g; second broth 3 to 15 g; and energy drink 1 to 5 oz.

34. The low-sodium fasting-mimicking diet composition of claim 21, wherein day-specific nutrient constraints comprise: for day 1, less than 30 g sugar, less than 28 g protein, 20 to 30 g monounsaturated fat and 6 to 10 g polyunsaturated fat; and for days 2 to 5 and any additional days, less than 20 g sugar, less than 18 g protein, 10 to 15 g monounsaturated fat, and 3 to 5 g polyunsaturated fat.

35. The low-sodium fasting-mimicking diet composition of claim 21, wherein day-1 caloric intake is 900 to 1300 kcal with 5 to 15% protein, 45 to 65% fat, and 25 to 45% carbohydrate, and days 2 to 5 provide 600 to 800 kcal with 5 to 15% protein, 35 to 55% fat, and 40 to 55% carbohydrate.

36. The low-sodium fasting-mimicking diet composition of claim 21, wherein potassium is present at less than about 1500 mg per day, less than about 1000 mg per day, less than about 800 mg per day, less than about 500 mg per day, or less than about 300 mg per day.

37. The low-sodium fasting-mimicking diet composition of claim 21, wherein phosphorus is present at less than about 1500 mg per day, less than about 1000 mg per day, less than about 800 mg per day, less than about 500 mg per day, or less than about 300 mg per day.
